(19) United States
(12) Patent Application Publication
(10) Pub. No.: US 2025/0345546 A1
(43) Pub. Date: Nov. 13, 2025

(54) ADJUSTMENT ASSEMBLY FOR HEADGEAR

(71) Applicant: Fisher & Paykel Healthcare Limited, Auckland (NZ)

(72) Inventors: Paul Mathew FREESTONE, Auckland (NZ); Jake Baker HOCKING, Auckland (NZ)

(21) Appl. No.: 19/127,726

(22) PCT Filed: Nov. 7, 2023
(86) PCT No.: PCT/NZ2023/050122

§ 371 Date: May 6, 2025

Related U.S. Application Data
(60) Provisional application No. 63/382,604, filed on Nov. 7, 2022.

(51) Int. Cl. A61M 16/06; A61M 16/08

(52) U.S. Cl. CPC A61M 16/0605; A61M 16/0683; A61M 16/0816

(57) ABSTRACT

Disclosed is an adjustment assembly for headgear for use in respiratory therapy, for example in the treatment of sleep apnea. The assembly includes an adjustment mechanism in communication with one or more tension elements, and actuation of the assembly may adjust the effective length or tension of the tension elements. Also disclosed are assemblies including an adjustment assembly and one or more of headgear, patient interfaces, seals, frames, and gas supply.

### BACKGROUND

### Field

The present disclosure relates to an adjustment assembly for headgear. In particular, the present disclosure relates to an assembly for adjusting the length or tension of a headgear. Also disclosed are assemblies including an adjustment assembly and one or more of headgear, patient interfaces, seals, frames, and gas supply.

### Description of Related Art

In assisted breathing, respiratory gases are supplied to a patient through a patient interface via one or more flexible breathing tubes. The patient interface may include a nasal cannula (contacting or extending into the nares), nasal mask (sealing around the nose), compact nasal mask (sealing around the nares and/or a lower part of the nose), nasal pillows (sealing around or on an inner side of each of the nares), oral mask (sealing around the mouth), full face mask (sealing around the nose and mouth), compact full face mask (combining an oral mask and a nasal mask or nasal pillows), a total face mask (sealing around the eyes, nose and mouth) endotracheal tube (inserted into the trachea through the mouth or nose), tracheostomy tube (inserted into the trachea through an incision in the neck), or other known types of interfaces. The patient interface is generally held in place on the head of the user by a headgear.

A function of the headgear arrangement may be to apply a sealing force between the patient interface and the patient, and/or to restrain the interface in response to forces typically encountered in use, such as blow-off forces and tube drag forces, to keep the interface in position against the face of the user. For therapies such as continuous positive airway pressure (CPAP) and non-invasive ventilation (NIV), the headgear arrangement is also generally required to maintain the patient interface in a sealing arrangement with the face, nose and/or mouth of the user.

Existing headgear involves strapping an interface to the face. This may involve looping each strap through a buckle and pulling the strap back on itself through the buckle, and then fastening the strap in place on itself using a hook-and-loop fastener, for example. Obtaining the appropriate level of tightness of the headgear is a balance between comfort and maintaining a good seal between the face and the interface. Some people may have a tendency to overtighten the headgear, in particular to reduce gas leakage, with the effect that too much force is applied. This may result in discomfort and a reduction in compliance (e.g., adherence to a prescribed therapy schedule). Achieving the right level of tension can be complicated by the need to manually tighten headgear straps. This can be difficult for people who have reduced dexterity. It may also inconveniently require separately tightening two or more straps. Further, it can be difficult to achieve the fine control required to achieve the appropriate tightness using straps and buckles.

### BRIEF SUMMARY

The disclosure relates to an adjustment assembly for headgear for use in respiratory therapy, for example in the treatment of sleep apnea. The adjustment assembly includes an adjustment mechanism in communication with one or more tension elements which may be connected to headgear. Actuation of the assembly may adjust the effective length or tension of the tension elements, and thus adjust the size, perimeter, circumference of the headgear or tension of the headgear against a user's head and face in use. Also disclosed are assemblies including an adjustment assembly and one or more of headgear, patient interfaces, seals, frames, and gas supply.

In a first aspect there is provided a headgear adjustment assembly for applying or releasing tension on tension elements connected to headgear, the headgear adjustment assembly comprising: one or more tension elements configured for engagement with a portion of a headgear; a rotatable member coupled with the one or more tension elements, wherein the rotatable member is configured to drive the one or more tension elements as the rotatable member is rotated; an actuator configured to selectively rotate the rotatable member to adjust tension in the headgear, in use; and a torque transfer mechanism configured to communicate torque between the actuator and the rotatable member, and configured to limit the torque applied to the rotatable member by the actuator, wherein the torque transfer mechanism includes a rotatable torque transfer element.

The assembly may be configured such that rotation of the actuator in a tightening direction drives rotation of the rotatable member via the torque transfer mechanism and a retracting action on the tension elements, such that the headgear is tightened.

The torque transfer element may be configured to limit the torque applied to the rotatable member by the actuator to a torque threshold.

The torque transfer element may be positioned in an interior cavity of the actuator, and may be rotatable in the interior cavity of the actuator.

The torque transfer element may be configured to move axially and rotationally with respect to the actuator.

The torque transfer element may be integrally formed with or rigidly connected to the rotatable member. For example, the torque transfer element may be connected to the rotatable member via a snap fit connection.

The torque transfer element may be positioned coaxially with the actuator, and/or with the rotatable member.

The torque transfer element may include a central cylindrical body, and one or more resilient arms. The one or more arms may be co-planar with one another. They may have a predetermined spring constant. They may be stiffer in the axial direction than the radial direction.

Each arm may include a proximal portion integrally formed with or rigidly connected to the central body, and a distal portion integrally formed with or rigidly connected to the proximal portion. The proximal portion may extend radially outwardly from the central body. The distal portion may extend substantially perpendicularly from the proximal portion.

The torque transfer element may be configured to be able to deform such that the distal portion of each arm moves radially inwardly towards the central body. Each arm may be cantilevered from the central body. The distal portion of the arm may include a radially outwardly projecting protrusion. The protrusion may be configured to interact with contours on the interior side wall of the actuator. For example, the protrusion is configured to interact with inwardly projecting protrusions on the actuator.

The interior side wall of the actuator may include a plurality of contours. The contours may be configured to contact the distal portion of the torque transfer element. Each arm may deform radially inwardly towards the central body by the contours on the interior side wall when the torque exerted by the actuator on each arm reaches a torque threshold, such that further rotation of the actuator does not communicate torque exceeding the torque threshold to the rotatable member.

The torque transfer element and interior cavity of the actuator may be sized such that each arm abuts the contours of the interior side wall of the actuator, in use. The interior side wall may include an inwardly projecting ramp including a sloping surface substantially perpendicular to the inner wall that runs between a forward part of the interior and a rear part of the interior.

The torque transfer element and interior cavity of the actuator may be sized such that the torque transfer element is able to move along its axis of rotation within the interior cavity.

The actuator may be configured to be directly actuated by a user. It may include an outer surface configured to be gripped by a user and rotated during use. For example, the outer surface may be configured to be gripped by a user's fingers. The outer surface may have a frustoconical shape. Alternatively, the outer surface may have an annular shape. The outer surface may include one or more concave impressions.

The actuator may be a unitary body, or may comprise two or more parts.

The actuator may be coupled to the rotatable member in the axial direction, such that axial translation of the actuator effects axial translation of the rotatable member.

Where the actuator comprises two or more parts, the actuator may comprise an inner part configured to engage with the torque transfer element and an outer part configured to be directly actuated by a user, the inner part and outer part in rotational communication with each other. The outer part may be translatable along its axis of rotation relative to the inner part. The outer part may be coupled to the rotatable member such that axial translation of the outer part effects axial translation of the rotatable member. Axial translation of the outer part may shift the first locking element away from the second locking element.

The actuator may include a rear opening through which the torque transfer element is rigidly connected to the rotatable member. The actuator may include a front end including an opening, and a covering cap engaged (e.g., sealingly engaged) with the front end of the actuator.

The rotatable member and the torque transfer element may be coaxial, i.e., have the same axis of rotation.

The rotatable member may include a front body and a rear body being rigidly connected or integrally formed with each other. The front body of the rotatable member may be rigidly connected to or integrally formed with the torque transfer element. The front body and rear body may be separated by one or more posts. The space between the posts may define one or more windows, where the windows may define a portion of a gas flow path. The rear body of the rotatable member may include a central opening defining a portion of a gas flow path. The rotatable member may include an annular channel into which the tension element may be wound, stored and/or unwound. The annular channel may be positioned on an outer wall of the rear body.

The headgear adjustment assembly may include a first locking element including a first set of teeth, such as rearwardly projecting teeth. The first locking element may be positioned on the rotatable member or the on the torque transfer element.

The first set of teeth may be circularly arranged. They may be equally spaced and sized. The first set of teeth may be asymmetric. They may be positioned around the central opening of the rotatable member.

The headgear adjustment assembly may include a base including a main body, and the main body may have an opening for the gas flow path. The base may include one or more side channels for receiving the tension element, the side channels may be positioned on opposing side arms of the main body. The base may include a forward projecting mount including at least one mount post extending forwards from the main body and a forward projection connected to a forward end of the at least one mount post.

The headgear adjustment assembly may include a second locking element including a second set of teeth, such as forward projecting teeth. The second locking element may be positioned on a front side of the base. The second set of teeth may be circularly arranged and equally spaced and sized. They may be uniformly slanted off-center. They may be positioned around the opening on the main body of the base. The first set and second set of teeth may be complementarily shaped such that they can intermesh when brought into contact.

The headgear adjustment assembly may include a first biasing element positioned in the actuator interior in operative communication with the torque transfer element. The first biasing element may be positioned on the actuator between the covering cap and the torque transfer element. It may be configured to bias the first locking element against the second locking element.

The headgear adjustment assembly may include a torsional biasing member configured to rotationally bias the rotatable member. The torsional biasing member may bias the rotatable member towards a state corresponding to the one or more tension elements being at least partially retracted.

The headgear adjustment assembly may include a housing including an interior for retaining the rotatable member, a side wall including a front end defining at least part of a front aperture and a rear end defining at least part of a rear aperture. The side wall may include an opening for a gas flow path and a connection member for connecting with a breathing gases tube. The gas flow path may be between the breathing gases tube and the main body opening. The gas flow path between the opening and the seal may be substantially airtight. The gas flow path may be in direct contact with at least one of the side wall opening, the torque transfer element, and the rotatable member. The headgear adjustment assembly may include a breathing gases tube configured to engage with the connection member on the housing.

The headgear adjustment assembly may include a seal configured to seal against a user's face, the seal including an interior cavity into which breathing gases may flow. The seal may surround a seal mount on the base, such that a portion of the seal mount projects into the interior cavity of the seal.

The tension element may be an elongate filament. The tension element may be a strand of flexible material, two or more strands braided, woven, or otherwise formed into a line, thread, ribbon, or tape. The tension element may include one or more of chain links, corrugations, notches, and ribs. The tension element may be substantially inextensible at tensions at or below normal operating forces (e.g., blow-off and tube-drag forces).

One or more end portions of the tension element may be engaged with the rotatable member. For example, the tension element may be engaged with the rotatable member such that each tension element is configured to wind or unwind about the rotatable member as the rotatable member is rotated.

The rotatable member may include a central body including an inner aperture. The inner aperture may define a gas flow path.

The headgear adjustment assembly may include a conduit for breathing gases defining a gases flow path. The conduit may be positioned in a central aperture of the base. The conduit may extend through apertures of the rotatable element and the torque transfer element. The conduit may separate the gases flow path from moving parts in the assembly, such as the actuator, the torque transfer element, and the rotatable member.

The rotatable member may drive the one or more tension elements in a tightening direction by winding the one or more tension elements about the rotatable member.

In a second aspect, there is provided a patient interface assembly including a headgear including one or more side straps, a top strap and a rear strap, and the headgear adjustment assembly as described. The headgear may include one or more pathways for receiving the tension element. The one or more pathways may be positioned along one or more side straps of the headgear. The one or more pathways may be positioned along two side straps of the headgear or positioned along four side straps of the headgear (e.g., in a four-point headgear). The one or more pathways may include a substantially rigid component, such as a thermoformed plastic element.

In a third aspect, there is provided a headgear adjustment assembly including: one or more tension elements connected to a portion of the headgear assembly; a rotatable member including a first locking element, the rotatable member coupled with the one or more tension elements, the rotatable member configured to control an effective length of the one or more tension elements as the rotatable member is rotated; an actuator configured to selectively rotate the rotatable member to adjust tension in the headgear assembly, in use; wherein the rotatable member is translatable along its axis of rotation between an engaged configuration in which the first locking element is in rotational communication with a second locking element, and a disengaged configuration in which the first locking element and the second locking element are not in rotational communication. The headgear adjustment assembly may include a housing including an interior configured to retain the rotatable member. The second locking element may be positioned on an inner wall of the housing.

The headgear adjustment assembly may include a rotatable torque transfer element configured to communicate torque between the actuator and the rotatable member, and may be configured to limit the torque applied to the rotatable member by the actuator.

In a fourth aspect, there is provided a headgear adjustment assembly including: one or more tension elements extending along at least a portion of a headgear; a pinion engaged with one or more tension elements, each of the one or more tension elements including a rack; a rotatable actuator configured to be actuated by a user; a rotatable torque transfer element that communicates rotational force between the actuator and the pinion, the torque transfer element configured to limit the torque applied to the rotatable member by the actuator to a torque threshold.

The assembly may include a first locking element including a first set of teeth. The first locking element may be positioned on the rotatable member or on the torque transfer element. The first set of teeth may be circularly arranged and equally spaced and sized. They may be uniformly slanted off-center. They may be positioned around the central opening of the rotatable member.

The headgear adjustment assembly may include a second locking element including a second set of teeth. The second locking element may be positioned on a front side of the base. The second set of teeth may be circularly arranged and equally spaced and sized. They may be uniformly slanted off-center. They may be positioned around the opening on the main body of the base. The first set and second set of teeth may be complementarily shaped such that they can intermesh when brought into contact.

In a fifth aspect, there is provided a headgear adjustment assembly including: one or more tension elements extending along a portion of a headgear assembly; a rotatable torque transfer element including a pinion and a first locking element, the pinion engaged with one or more tension elements, each of the one or more tension elements including a rack; an actuator configured to selectively rotate the rotatable member to adjust tension in the headgear assembly, in use; a second locking element positioned on a mount connected to a headgear strap; wherein the torque transfer element is translatable along its axis of rotation between an engaged configuration in which the first locking element and the second locking element are engaged, and a disengaged configuration in which the first locking element and the second locking element are disengaged.

In a sixth aspect, there is provided a headgear adjustment assembly, the assembly including: one or more tension elements connected to a portion of a headgear; an actuator configured to be rotated by a user; a rotatable member including a first locking element, the rotatable member coupled with the one or more tension elements, where each tension element is configured to wind or unwind about the rotatable member as the rotatable member is rotated; a housing including an interior retaining the rotatable member and including indentations on an interior wall; and a rotatable torque transfer element for communicating torque between the actuator and the rotatable member, including: a central body and one or more arms extending radially outwardly from the central body, each arm including a proximal portion connected to the central body and a distal portion configured to interact with the indentations; and a second locking element that is engageable with the first locking element, wherein engagement between the first and second locking element effects rotational communication between the torque transfer element and the rotatable member; wherein the torque transfer element is translatable between an engaged configuration in which the first locking element and the second locking element are in rotational communication, and a disengaged configuration in which the first locking element and the second locking element are not in rotational communication.

The torque transfer element may be translatable along its axis of rotation. The distal portion may be translatable between a first section and a second section the inner indentations, where the first section and a second section have different shape and/or Size. The first section may be configured to prevent rotation of the distal portion of the torque transfer element relative to the housing, and the second section is configured to enable rotation of the distal portion of the torque transfer element relative to the housing in one rotational direction but not in the opposite rotational direction. The distal portion of the torque transfer element may be configured to deform against the side wall of the second section under torque and slip between contiguous indentations in one rotational direction.

The rotatable member may be rotationally biased in a tightening direction. The adjustment assembly may include a torsional biasing member connecting the rotatable member and the housing.

The first locking element and second locking element may each include a plurality of reversibly engageable teeth. In some examples, the first locking element may alternatively comprise one or more resilient pawls. The one or more pawls may be configured to deform against the second locking element when the torque exerted by the actuator on each pawl reaches a torque threshold, such that further rotation of the actuator does not communicate torque exceeding the torque threshold to the rotatable member.

The headgear adjustment assembly may comprise a biasing member to rotationally bias the rotatable member in a tightening direction. The biasing member may bias the rotatable member towards a state corresponding to the one or more tension elements being at least partially retracted. The biasing member may be a torsional spring.

In some examples, the actuator may comprise an input member configured to receive a torque from the user's rotation of the actuator; and an output member in rotational communication with the torque transfer element; wherein the input member and output member are rotationally coupled up to a torque threshold, and rotationally decoupled above the torque threshold. The input member and output member may be frictionally coupled up to a torque threshold, and frictionally decoupled above the torque threshold. The input member and the output member may be in contact at an interface. The interface is substantially planar, or non-planar. Where the interface is non-planar it may comprise a plurality of formations.

In a seventh aspect, there is provided a patient interface assembly for supplying a flow of pressurized breathing gases to an airway of a patient, the patient interface assembly comprising: a patient interface; a headgear assembly configured to secure the patient interface to the patient; and the headgear adjustment assembly as described.

In an eighth aspect, there is provided a patient interface assembly for supplying a flow of pressurized breathing gases to an airway of a patient, the patient interface assembly comprising: a patient interface comprising: a frame comprising an inlet configured to receive the flow of pressurized breathing gases; and a cushion attached or attachable to the frame and configured to receive the flow of pressurized breathing gases from the frame and supply the flow of pressurized breathing gases to an entrance or entrances to the airway of the patient; a headgear assembly comprising: a pair of side strap portions configured to attach to the frame and extend across a side of the patient's face; a back strap portion extending between the pair of side strap portions and configured to extend around a back of the head of the patient; a top strap portion extending between the pair of side strap portions and configured to extend over a top of the head of the patient; and a tension element associated with at least one of the pair of side strap portions, the back strap portion and/or the top strap portion; and a headgear adjustment assembly coupled with the tension element and comprising a rotary actuator operable by the patient to selectively adjust an effective length of the tension element, the headgear adjustment assembly comprising a clutch mechanism configured to limit a tensile force applied to the tension element.

The headgear adjustment assembly may include a spool coupled with the rotary actuator, and the clutch mechanism comprising a first ratchet mechanism configured to: permit rotation of the spool by the actuator in a first direction to wind the tension element onto the spool, and inhibit rotation of the spool in a second direction.

The clutch mechanism may include a cam mechanism configured to disengage the first ratchet mechanism when the actuator is rotated in the second direction, to permit rotation of the spool in the second direction. The clutch mechanism may include a second ratchet mechanism configured to permit the rotary actuator to rotate in the first direction with respect to the spool to limit the tensile force applied to the tension element.

In a ninth aspect, there is provided a patient interface assembly for supplying a flow of pressurized breathing gases to an airway of a patient, the patient interface assembly comprising: a patient interface comprising: a frame comprising an inlet configured to receive the flow of pressurized breathing gases; and a cushion attached or attachable to the frame and configured to receive the flow of pressurized breathing gases from the frame and supply the flow of pressurized breathing gases to an entrance or entrances to the airway of the patient; a headgear assembly comprising: a pair of upper side strap portions; a pair of lower side strap portions; a first tension element associated with the pair of upper side strap portions; and a second tension element associated with the pair of lower side strap portions; a first adjustment assembly coupled with the first tension element; a second adjustment assembly coupled with the second tension element; wherein the first second adjustment assembly and second adjustment assembly each comprise a rotary actuator operable by the patient to selectively adjust an effective length of the respective first and second tension elements, the headgear adjustment assembly comprising a clutch mechanism configured to limit a tensile force applied to the respective first and second tension elements.

In a tenth aspect, there is provided a headgear adjustment assembly including: one or more tension elements configured to be connected to a portion of a headgear assembly; a rotatable member coupled with the one or more tension elements and configured to control an effective length of the one or more tension elements as the rotatable member is rotated; an input member configured to receive a torque from a user; and an output member rotationally coupled with the rotatable member; wherein the input member and output member are rotationally coupled up to a torque threshold, and rotationally decoupled above the torque threshold.

The input member and output member may be frictionally coupled up to the torque threshold, and frictionally decoupled above the torque threshold. The input member may contact the output member at torques above and below the torque threshold.

In some examples, there may be no deformation of the input member or the output member above and below the torque threshold.

The input member and the output member may be in contact at an interface. The interface may comprise an inner surface of the input member and an outer surface of the output member. The interface may have a predetermined frictional force, or a predetermined frictional coefficient. The torque threshold may be determined by the frictional coefficient or the frictional force at the interface.

The interface may comprise a surface of the input member in contact with a surface of the output member. A portion of the interface may on a plane that is substantially perpendicular to an axis of the rotation.

The interface may be substantially smooth and/or planar. Alternatively, the interface may be non-planar, and may comprise a plurality of formations. In some examples, the first surface and second surface may comprise a plurality of intermeshing formations.

Further aspects, novel features and advantages of the present disclosure will be readily apparent to those skilled in the art in light any one or more of the illustrative examples set out in the detailed description and drawings. The description and drawings are to be regarded as illustrative in nature, and not restrictive. Modifications or improvements may be made without departing from the spirit or scope of the disclosure and claims.

### BRIEF DESCRIPTION OF THE DRAWINGS

A number of examples will now be described by way of example with reference to the accompanying drawings, in which:

FIG. 1 is an example respiratory therapy system in which headgear adjustment assemblies, headgear, and/or patient interfaces according to the present disclosure may be used.

FIG. 2 is an exploded isometric view of an example of the headgear adjustment assembly according to the First Example.

FIG. 3 is an exploded isometric view of the headgear adjustment assembly according to the First Example.

FIG. 4 is an isometric view of a torque transfer element according to the First Example.

FIG. 5 is an isometric view of a rotatable member according to the First Example.

FIG. 6 is a front view of a base according to the First Example.

FIG. 7 is a side view of a base according to the First Example.

FIG. 8 is an isometric view of an actuator, rotatable member, face plate and base according to the First Example.

FIG. 9 is a perspective view of an actuator according to the First Example.

FIG. 10 is a front perspective view of an actuator according to the First Example.

FIG. 11 is a front view of an actuator according to the First Example.

FIG. 12 is a view of a covering cap according to the First Example.

FIG. 13 is an isometric view of a housing according to the First Example.

FIG. 14 is an isometric view of a headgear adjustment assembly, seal, and headgear according to the First Example.

FIG. 15 is a schematic drawing of tension elements according to the First Example.

FIG. 16 is a top view photograph of tension elements according to the First Example.

FIG. 17 is an isometric view of a face plate according to the First Example.

FIG. 18 is an isometric view of a headgear showing a tension element pathway according to the First Example.

FIG. 19 is an isometric view of a headgear showing a tension element pathway according to the First Example.

FIG. 20 is an isometric view of a headgear showing a tension element pathway according to the First Example.

FIG. 21 is an isometric view of a headgear showing a tension element pathway according to the First Example.

FIG. 22 is an isometric view of a headgear showing a tension element pathway according to the First Example.

FIG. 23 is an isometric view of a headgear showing a tension element pathway according to the First Example.

FIG. 24 is an isometric view of a headgear showing a tension element pathway according to the First Example.

FIG. 25 is an isometric view of a headgear showing a tension element pathway according to the First Example.

FIG. 26 is an isometric view of a headgear adjustment assembly, seal and breathing gases tube according to the Second Example.

FIG. 27 is an isometric exploded view of a headgear adjustment assembly, seal, and breathing gases tube according to the Second Example.

FIG. 28 is an isometric view of a torque transfer element and rotatable member according to the Second Example.

FIG. 29 is an isometric view of a torque transfer element, and rotatable member in the engaged configuration with a base according to the Second Example.

FIG. 30 is a front perspective view of an actuator according to the Second Example.

FIG. 31 is an isometric view of a housing according to the Second Example.

FIG. 32 is a side view of a housing according to the Second Example.

FIG. 33 is an isometric view of a base according to the Second Example.

FIG. 34 is a rear perspective view of a base according to the Second Example.

FIG. 35 is an isometric exploded view of a headgear adjustment assembly, seal, breathing gases tube and gases flow path according to the Third Example.

FIG. 36 is an isometric view of a torque transfer element and rotatable member according to the Third Example.

FIG. 37 is a wireframe diagram of a torque transfer element and rotatable member according to the Third Example.

FIG. 38 is a rear view of an actuator according to the Third Example.

FIG. 39 is an isometric view of a torque transfer element and rotatable member in engagement with a first and second rack portion of first and second tension elements according to the Third Example.

FIG. 40 is an isometric view of first and second rack portions of first and second tension elements according to the Third Example.

FIG. 41 is a front view of a base according to the Third Example.

FIG. 42 is an isometric view of a base according to the Third Example.

FIG. 43 is an isometric view of a face plate according to the Third Example.

FIG. 44 is a rear view of a face plate according to the Third Example.

FIG. 45 is an isometric view of a headgear adjustment assembly, seal, breathing gases tube and headgear according to the Third Example.

FIG. 46 is an isometric exploded view of a headgear adjustment assembly according to the Fourth Example.

FIG. 47 is a top view of a lower body according to the Fourth Example.

FIG. 48 is a bottom view of an upper body according to the Fourth Example.

FIG. 49 is a side view of side arms and engagement element according to the Fourth Example.

FIG. 50 is an isometric view of side arms and engagement element according to the Fourth Example.

FIG. 51 is an isometric view of an actuator according to the Fourth Example.

FIG. 52 is an isometric view of a torque transfer element and rotatable member according to the Fourth Example.

FIG. 53 is a side view of a torque transfer element and rotatable member according to the Fourth Example.

FIG. 54 is a bottom view of a torque transfer element and rotatable member according to the Fourth Example.

FIG. 55 is an isometric view of a headgear adjustment assembly according to the Fourth Example.

FIG. 56 is a perspective cross section of a headgear adjustment assembly according to the Fourth Example.

FIG. 57 is a perspective view of a headgear and headgear adjustment assembly according to the Fourth Example.

FIG. 58 is an exploded isometric view of a headgear adjustment assembly according to the Fifth Example.

FIG. 59 shows side, bottom, and top views of an actuator according to the Fifth Example.

FIG. 60 is a perspective exploded view of an actuator and torque transfer element according to the Fifth Example.

FIG. 61 is a side view of a torque transfer element according to the Fifth Example.

FIG. 62 is a perspective view of a rotatable member according to the Fifth Example.

FIG. 63 is a perspective exploded view of a rotatable member and a torque transfer element according to the Fifth Example.

FIG. 64 is a perspective view of a base according to the Fifth Example.

FIG. 65 shows the interrelationship between the distal protrusion of the torque transfer element and the upper and lower sets of indentations on a base according to the Fifth Example.

FIG. 66 shows a schematic of an upper, lower, and intermediate zone of indentations in a base according to the Fifth Example.

FIG. 67 is a perspective view of a cover and rotatable member according to the Fifth Example.

FIG. 68 is a side cross section view of a headgear adjustment assembly in an engaged configuration according to the Fifth Example.

FIG. 69 is a side cross section view of a headgear adjustment assembly in a disengaged configuration according to the Fifth Example.

FIG. 70 is a side cross section view of a headgear adjustment assembly in a disengaged configuration according to the Fifth Example.

FIG. 71 is a side cross section view of a headgear adjustment assembly in a disengaged configuration according to the Fifth Example.

FIG. 72 is a perspective cross section view of a headgear adjustment assembly in a disengaged configuration according to the Fifth Example.

FIG. 73 is a perspective cross section view showing operation of a headgear adjustment assembly in a transitional configuration according to the Fifth Example.

FIG. 74 is a perspective cross section view showing operation of a headgear adjustment assembly in an engaged configuration according to the Fifth Example.

FIG. 75 is a perspective cross section view showing a tightening operation of a headgear adjustment assembly in an engaged configuration according to the Fifth Example.

FIG. 76 is a perspective cross section view showing operation of a headgear adjustment assembly in a transitional configuration according to the Fifth Example.

FIG. 77 is a perspective cut-away cross section view showing operation of a headgear adjustment assembly in a disengaged configuration according to the Fifth Example.

FIG. 78 is a perspective cut-away cross section view showing operation of a headgear adjustment assembly in a disengaged configuration according to the Fifth Example.

FIG. 79 is a perspective view of parts of an adjustment assembly according to the Fifth Example.

FIG. 80 is a perspective view of parts of an adjustment assembly according to the Fifth Example.

FIG. 81 is a perspective cut-away view of parts of an adjustment assembly according to the Fifth Example.

FIG. 82 is a perspective cut-away view of parts of an adjustment assembly according to the Fifth Example.

FIG. 83 is a perspective cut-away view of parts of an adjustment assembly according to the Fifth Example.

FIG. 84 is a perspective cut-away view of parts of an adjustment assembly according to the Fifth Example.

FIG. 85 is a perspective cut-away view of parts of an adjustment assembly according to the Fifth Example.

FIG. 86 is a perspective cut-away view of parts of an alternative example of an adjustment assembly according to the Fifth Example.

FIG. 87 is a side cross-section view of parts of an alternative example of an adjustment assembly according to the Fifth Example.

FIG. 88 is an exploded view of parts of an alternative example of an adjustment assembly according to the Fifth Example.

FIG. 89 is an exploded view of parts of an alternative example of an adjustment assembly according to the Fifth Example.

FIG. 90 is a perspective cut-away view of parts of an alternative example of an adjustment assembly according to the Fifth Example.

FIG. 91 is a side cross-section view of an alternative example of an adjustment assembly in an engaged configuration according to the Fifth Example.

FIG. 92 is a side cross-section view of an alternative example of an adjustment assembly in a disengaged configuration according to the Fifth Example.

FIG. 93 is an exploded view of parts of an alternative example of an adjustment assembly according to the Fifth Example.

FIG. 94 is an exploded view of parts of an alternative example of an adjustment assembly according to the Fifth Example.

FIG. 95 is an exploded cross section view of an alternative example of an adjustment assembly according to the Fifth Example.

FIG. 96 is a perspective cut-away view of parts of an alternative example of an adjustment assembly according to the Fifth Example.

FIG. 97 is a side view of parts of an alternative example of an adjustment assembly in an engaged configuration according to the Fifth Example.

FIG. 98 is a side view of parts of an alternative example of an adjustment assembly in a disengaged configuration according to the Fifth Example.

FIG. 99 is a side cross-section view of an alternative example of an adjustment assembly in an engaged configuration according to the Fifth Example.

FIG. 100 is an exploded view of an adjustment assembly according to the Sixth Example.

FIG. 101 is a perspective view of parts of an adjustment assembly according to the Sixth Example.

FIG. 102 is a perspective cross-section view of parts of an adjustment assembly according to the Sixth Example.

FIG. 103 is an exploded view of an adjustment assembly according to the Sixth Example.

FIG. 104 is a perspective view of parts of an adjustment assembly according to the Sixth Example.

FIG. 105 is a perspective cross-section view of parts of an adjustment assembly according to the Sixth Example.

FIG. 106 is a front view of a patient interface assembly comprising an adjustment assembly as described herein.

FIG. 107 is a perspective view of a patient interface assembly comprising an adjustment assembly as described herein.

FIG. 108 is a front view of a patient interface assembly comprising an adjustment assembly as described herein.

FIG. 109 is a front view of a patient interface assembly comprising an adjustment assembly as described herein.

FIG. 110 is a front view of a patient interface assembly comprising an adjustment assembly as described herein.

### DETAILED DESCRIPTION

To easily identify the discussion of any particular element shown in the accompanying drawings, the most significant digit or digits in the associated reference numeral refers to the figure number in which that element is first introduced. A single reference numeral may refer to the same, a similar, or an equivalent element in two or more examples. For brevity, the detailed description of that element in relation to one of those examples is intended to apply equally in relation to any other examples comprising that element, except as described or otherwise clearly apparent from the context.

Unless the context clearly requires otherwise, throughout the description and claims the words “comprising,” “including” and variants are to be construed in an inclusive sense rather than an exclusive sense. That is, such terms are to be construed in the sense of “including, but not limited to.”

The terms “forward”, “rear”, “top”, “bottom”, “upper”, “lower”, “above”, “below”, “up”, “down”, and other similar terms should be understood as descriptive of the relative relationship between depicted features in the figures and not limiting on the claims, especially relating to the adjustment assemblies and patient interface assemblies described herein, which may be operated in any orientation.

Any reference to publications or products throughout this specification, including the background, should in no way be considered as an admission that the publication or product is prior art, analogous, widely known or forms part of common general knowledge in the field.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

As used herein the term “and/or” means “and” or “or”, or both.

As used herein “(s)” following a noun means the plural and/or singular forms of the noun.

To those skilled in the art to which the disclosure relates, many changes in construction and widely differing examples and applications will suggest themselves. The descriptions and drawings disclosed herein are purely illustrative and are not intended to be in any sense limiting.

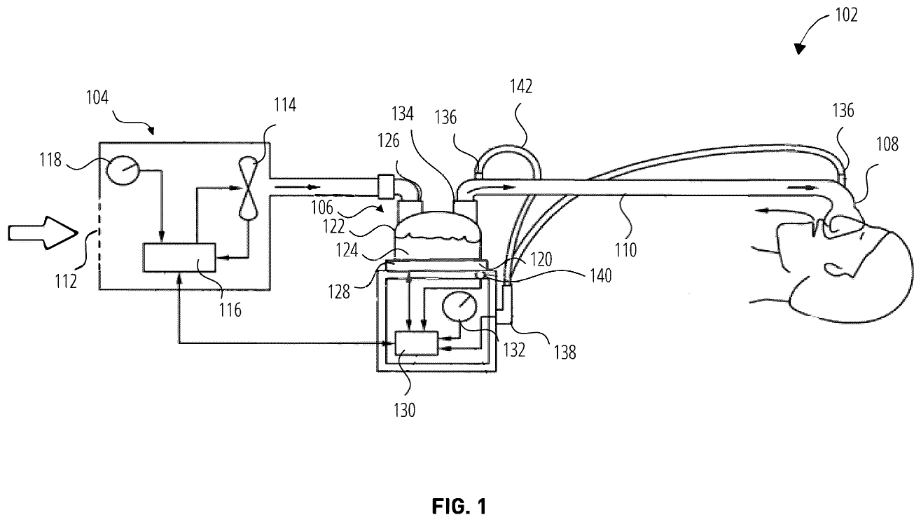

FIG. 1 illustrates an example respiratory therapy system 102 suitable for supplying breathing gases to a user for positive airway pressure (PAP) therapy (e.g., continuous positive airway pressure (CPAP) therapy or non-invasive ventilation (NIV) therapy). The example respiratory therapy system 102 may include a gas source 104, a humidifier 106, a patient interface assembly 108 and a breathing gas circuit 110 that connects the humidifier 106 (or gas source 104) to the patient interface assembly 108 The gas source 104 can provide a supply of breathing gas to the humidifier 106. The gas source 104 may comprise a blower in which breathing gas, e.g., ambient air, is drawn into the gas source 104 through an inlet 112 in the gas source 104 by an impeller 114. The rotational speed of the impeller 114 may be modulated to regulate the pressure of the breathing gas delivered to the patient. Breathing gas may include any single gas or multiple gases that are breathable by a user of the respiratory therapy system 102.

The pressure and/or flow rate of breathing gas exiting the gas source 104 may be regulated by a controller 116. The controller 116 may modulate the rotational speed of the impeller 114 according to one or more predetermined algorithms and in accordance with one or more user inputs that may be provided via a user input 118.

The gas source 104 represents an actively controlled flow generator. Other gas sources, such as a compressed air cylinder with suitable pressure or flow regulation, may alternatively, or additionally, be used to supply breathing gas. The outlet of the gas source 104 may be coupled to a separate humidifier 106, as shown. The humidifier 106 may be configured to heat and/or humidify the breathing gas prior to delivery, e.g., delivery to the user. In some examples, the humidifier 106 may be integrated with the gas source 104. The humidifier 106 may include a base 120 and a humidifier chamber 122. The humidifier chamber 122 may be configured to hold humidification fluid 124, such as water, and may be disengaged, e.g., temporarily disengaged or permanently disengaged, from the humidifier base 120 to allow it to be filled or replaced. The humidifier 106 receives gases from the gas source 104 through chamber inlet 126. The humidifier base 120 can include a heater such as a heater plate 128. The humidifier chamber 122 rests on the heater plate 128 when engaged with the humidifier base 120. The heater plate 128 dissipates heat, e.g., heat generated by electrical resistance, to the humidifier chamber 122. The humidifier chamber 122 preferably has a heat conductive base to enable the heat generated by the heater plate to pass efficiently to the humidification fluid 124. Controller 116 can also control the humidifier 106, and in particular the supply of electrical energy to the heater plate 128, to regulate any function of the humidifier 106, e.g., the temperature and humidity of the breathing gas supplied to the user. Alternatively, humidifier 106 may be controlled by a separate controller 130 according to one or more predetermined algorithms and/or in accordance with one or more user inputs that may be provided via a user input 132.

The breathing gas can be supplied to the user via a chamber outlet 134 and breathing gas circuit 110 in the form of a conduit which may incorporate a heating or warming element (not shown), e.g., a heater wire, to heat or warm (e.g., keep hot or warm) the breathing gases during transportation to the patient interface assembly 108. The electrical energy supplied to the heater wire may be controlled by the controller 116 or controller 130. The controller 116 may receive feedback from one or more sensors incorporated in a control network throughout the respiratory therapy system to monitor properties of the breathing gas, such as, but not limited to, any one or more of pressure, flow, temperature, and/or humidity. For example, the respiratory therapy system 102 may include gas flow rate sensors 136 which are connected through a connector 138 and which can communicate with the controller 130. A heater plate temperature sensor 140 can communicate with the controller 130.

The patient interface assembly 108 couples the user with the respiratory therapy system 102, such that gases, e.g., heated, and humidified gases from the humidifier 106, may be delivered to the user's respiratory system. Breathing gases can be delivered to the user at, or near, optimal temperature and humidity (e.g., warmed and fully saturated with water vapor at temperatures of between 27 and 37° C.) as the gases are delivered to the user's nares and/or mouth. Emulating the conditions within healthy adult lungs (37° C., 44 mg/L humidity) can help maintain healthy mucociliary function in users with respiratory disorders affecting secretion and for all patients humidifying the gas may help maintain comfort and compliance. A number of different styles of patient interface assembly 108, such as those disclosed herein, may be used in the example respiratory therapy system 102 or a similar system.

In the illustrated respiratory therapy system, the breathing gas circuit 110 comprises a single limb circuit, i.e., an inspiratory conduit. In other examples, which may be used for NIV therapy for example, the breathing gas circuit may comprise a dual-limb circuit. A Y-piece may be provided between the inspiratory conduit, the patient interface, and an expiratory conduit provided to convey expired gases and excess inspiratory gases from the Y-piece to a return inlet of the gas source. The expiratory conduit may include a heating or warming element, e.g., a heater wire. Alternatively, or additionally, the expiratory conduit may be formed from a breathable material.

Described herein are examples of adjustment assemblies for patient interfaces and/or headgear arrangements.

The First, Second, Third, Fourth, Fifth and Sixth Examples described below relate to adjustment assemblies for headgear, optionally with headgear, patient interfaces, seals, frames, and gas supply. The adjustment assemblies may generally include: an actuator for controlling the assembly and adjusting the tightness of the headgear, where the actuator may be configured to be user-operable and may be rotatable; a rotatable member in communication with the actuator configured to drive one or more tension elements as the rotatable member is rotated; and a torque transfer mechanism configured to communicate torque between the actuator and the rotatable member.

The adjustment assemblies described below are exemplified in a particular configuration with respect to headgear and/or a patient interface. For example, the adjustment assembly may be located on a patient interface, on a headgear strap, or on a gases supply tube. Similarly, the adjustment assembly may be integrated with a patient interface, headgear strap, or gases supply tube. However, the adjustment assembly may be configured to be located on any part of the headgear, patient interface, tubing, or straps.

The adjustment assemblies are in operative communication with headgear such that actuation of the adjustment assembly translates to an adjustment of the headgear tension, in use.

The adjustment assembly may be configured to prevent or reduce the risk of overtightening a patient interface assembly by comprising a clutch mechanism that limits the amount of torque transferred by a user onto tension elements and headgear straps. The assembly may comprise a clutch mechanism between the user-actuated input and the rotatable member coupled with one or more tension elements that extend to headgear straps. The rotatable member is configured to control an effective length of the one or more tension elements as the rotatable member is rotated. The clutch mechanism may comprise an input member configured to receive a torque from a user and an output member rotatably coupled with the rotatable member, wherein the input member and output member are rotationally coupled up to a torque threshold, and rotationally decoupled above the torque threshold.

Assemblies may further include features and combinations of features described below.

### FIRST EXAMPLE

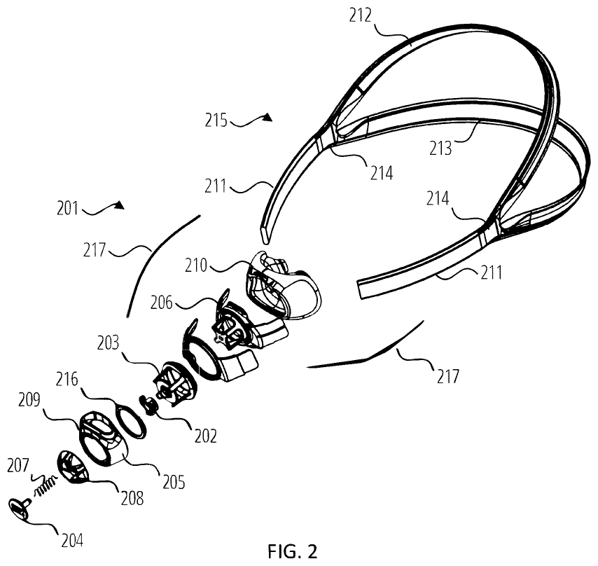

With reference to FIG. 2 to FIG. 25, there is shown a First Example of an adjustment assembly 201 for applying and or releasing tension on tension elements connected to headgear. Adjustment assembly 201 is in operative communication with headgear such that actuation of the adjustment assembly 201 translates to an adjustment of the headgear tension, in use.

The adjustment assembly 201 may include an actuator 208, torque transfer element 202, rotatable member 203, base 206 and housing 205. Broadly, the actuator 208 and rotatable member 203 are in rotational communication via torque transfer element 202. The torque communicated from the actuator 208 to the rotatable member 203 is limited to a torque threshold by the torque transfer element 202. Where the actuator 208 is rotated against the torque transfer element 202 with torque exceeding the torque threshold, the torque transfer element 202 deforms such that torque exceeding the torque threshold is not transferred to the rotatable member 203. The rotatable member 203 is connected to one or more tension elements which are connected to a headgear. The effective length of the one or more tension elements—that is, the length of the part of the tension element transferring forces between the headgear and adjustment assembly-is therefore controlled by the rotatable member.

The effective length of the tension element, and thus the tension on the tension element in use, may be selectively adjusted by a user whilst avoiding or minimizing a risk of overtightening.

### Actuator

An actuator 208 may be positioned on a front part of the adjustment assembly 201, configured to be directly actuated by a user, and in particular configured to be selectively rotated clockwise and counter-clockwise by a user's fingers. In this way, the actuator 208 may be configured as a dial. The actuator 208 may be configured to provide sensory feedback to the user, for example, in the form of audible or tactile feedback during adjustment. The actuator may be configured to provide tactile or audible feedback in conjunction with incremental rotation of the actuator 208. As tightening of a headgear for respiratory therapy can require fine control over the forces exerted on the straps and the degree to which the straps are tightened, there may benefit in the use of a finger-actuated actuator 208. The ability to tighten a headgear using a finger-actuated rotational movement allows very fine control over the degree of tightening, making it easier for a user to achieve the correct tension in the headgear.

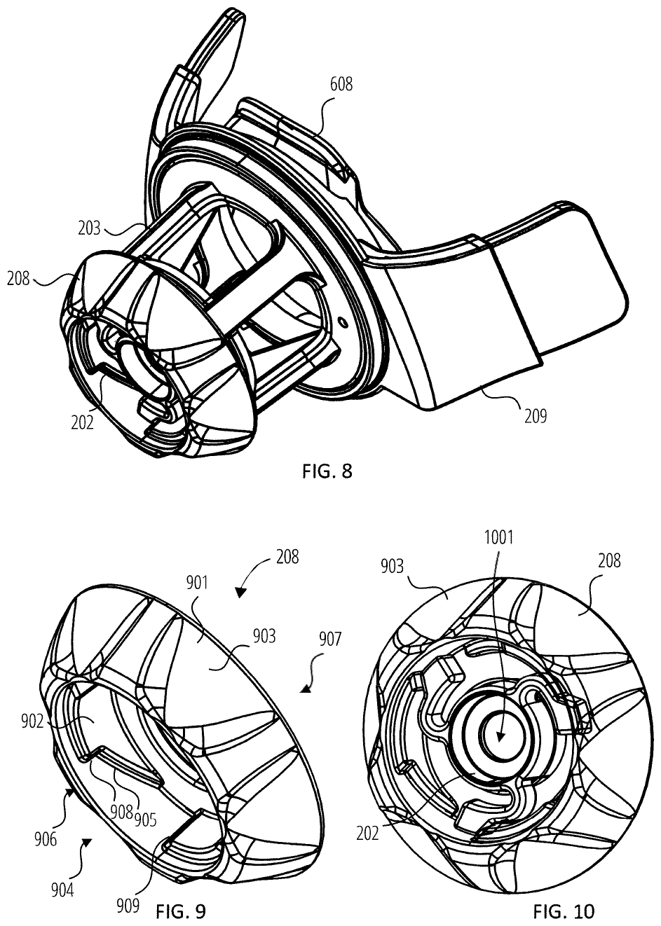

With particular reference to FIG. 9 to FIG. 11, the actuator 208 includes an outer surface 901, and a central recess defining an interior including an interior wall 902. The outer surface 901 may have a generally annular or frustoconical profile. Gripping features 903 are located on the outer surface 901 to assist the user to locate the actuator 208 by touch and also to assist the user to maintain an adequate grip on the actuator 208 when tightening or untightening the tension elements. Gripping features shown in FIG. 9 include a plurality of concave impressions or scallops located on the outer surface 901 of the actuator 208. Alternative gripping features 903 may alternatively or additionally include: one or more concave features positioned along a middle portion of the outer surface 901, such that the diameter of the outer surface 901 is lower towards the middle and greater towards front and rear portions of the outer surface 901, and/or a plurality of ribs which may be positioned longitudinally or circumferentially on the outer surface 901. The outer surface 901 may be textured (e.g., knurled) or smooth to the touch. Grip of the gripping features 903 can be increased by construction of, or application to, the outer surface 901 a soft and/or pliable material and/or material including a relatively higher coefficient of friction compared to a hard plastic material, which can be more easily gripped by a user's fingers, such as a soft polymer.

The actuator 208 includes an interior at least partially defined by a front opening 904, a rear opening 1001, and one or more interior walls 902. The interior is configured to at least partially house the torque transfer element 202. A rear wall 1101 of the actuator 208 includes rim projecting inwardly from the rear end of the interior wall 902. The rear wall 1101 includes the rear opening 1001 at a central position, such that the rear wall 1101 is an annular rim or flange about the central rear opening 1001. Thus the rear wall 1101 serves to retain the torque transfer element 202 within the interior of the actuator 208 and the rear opening 1001 serves to allow the communication of the torque transfer element 202 with parts positioned to the rear of the actuator, such as a rotatable member 203.

The interior wall 902 includes at least one inwardly protruding contour configured to interact with the torque transfer element 202. The contour includes a ramp 905 along a portion of the interior wall 902. The ramp 905 is sloped from a front end 906 to a rear end 907 along the interior wall 902. A function of the ramp 905 is to translate rotational movement of the actuator 208 to axial movement of the torque transfer element 202 in the interior cavity, as described in further detail below.

A front stop 908 is positioned at or near the front end 906 of the ramp 905, the front stop 908 including a flat face for abutment with the torque transfer element 202, as is described in further detail below. The face of the front stop 908 may be at right angles to the surface of the interior wall 902, or angled or sloped away from right angle. The face of the front stop 908 may have the same depth, in the radial or axial direction, as that of the ramp 905, or it may have a slightly greater or lesser depth.

The rear end 907 of the ramp 905 may be near to or aligned with the rear wall of the interior cavity. A rear stop 909 is positioned at or near the rear end of the ramp 905, or the rear stop 909 may be separated from the rear end of the ramp 905 by a radially flat section located between the rear end of the ramp 905 and the rear stop 909. The rear stop 909 may be configured as a flat face. The face of the rear stop 909 may be angled or sloped slightly away from the normal (relative to a radial line from the central axis). The face of the rear stop 909 may include chamfered or filleted portions.

The ramp 905, front stop 908 and rear stop 909 may be provided as a unitary projection on the interior wall 902. Alternatively, two of the ramp 905, front stop 908 and rear stop 909 may appear as unitary projections, or each of the ramp 905, front stop 908 and rear stop 909 may appear as separate projections.

The ramp 905, front stop 908 and rear stop 909 may be integrally formed with the body of the actuator 208. Alternatively, any of the ramp 905, front stop 908 and rear stop 909 may be separate to, and rigidly connected to, the interior wall 902 of the actuator 208.

### Torque Transfer Element

The torque transfer element 202 operationally connects the actuator 208 and rotatable member 203. It is configured to communicate torque from the actuator 208 to the rotatable member 203, and to limit the amount of torque transferred to the rotatable member 203.

With particular reference to FIG. 4 and FIG. 10, the torque transfer element 202 is positioned in the interior of the actuator 208, and the actuator 208 and torque transfer element 202 share the same axis of rotation.

The torque transfer element includes a central body 401 and one or more arms 402 extending generally radially from the central body 401. The central body 401 includes a generally cylindrical body to which the one or more arms 402 are attached. The central body 401 may include a central aperture 403. The central body 401 may include a connector, such as a snap fit connector, for connecting to the rotatable member 203. Alternatively, the central body 401 may be integrally formed with the rotatable member 203.

Each arm 402 includes a proximal portion 404 integrally formed with, or rigidly connected to, the central body 401, and a distal portion 405 integrally formed with, or rigidly connected to, the proximal portion 404.

The distal portion 405 may be offset from the proximal portion in a circumferential direction. For example, each arm 402 has a leading side and a trailing side, the leading side and trailing side being the forward and rear side of the arm, respectively, when rotated in a tightening direction. Each arm 402 may be substantially arcuate, or include a substantially arcuate portion. The proximal portion 404 extends radially outwardly from the central body 401, and the distal portion 405 may be oriented at an angle with respect to the proximal portion 404. For example, the distal portion 405 may be oriented from the proximal portion 404 at an angle of approximately 90degrees.

The distal portion 405 trails the proximal portion 404 in the untightening direction giving the distal portion 405 an appearance of being swept back from the proximal portion 404. With reference to FIG. 4, for example, the torque transfer element 202 is configured to be rotated in a clockwise direction by the actuator 208 to tighten the headgear. The distal portion 405 of each arm 402 extends from the proximal portion 404 in the clockwise direction. The distal portion 405 trails the proximal portion 404, and has a swept back appearance, in the anticlockwise direction.

The distal portion 406 may have a curvature along its length. The trailing side of the distal portion 405 is substantially equidistant from an outer side surface of the central body 401 along its length. The arm 402 may include a curvature on its leading side of about approximately 90 degrees defining the transition between the proximal portion 404 and distal portion 405.

Accordingly, each arm 402 is cantilevered from the central body 401. The proximal portion 404 cantilevers radially from the central body 401, and the distal portion 405 cantilevers substantially perpendicularly from the proximal portion 404.

The distal portion 405 is configured for engaging with the interior contours of the actuator 208. To better engage with the actuator 208, the arm 402 includes a distal protrusion 406 that projects radially outwardly from the distal portion 405 and is configured to engage with the contours of the interior wall 902 of the actuator 208. The distal protrusion 406 has a leading side corresponding with the leading side of the arms 402. The leading side of the distal protrusion 406 is configured to interact with contours on the interior of the actuator 208, and is shaped and sized so as to tune the desired amount of torque required by the actuator 208 to cause deformation of the arm 402.

The arms 402 provide interference with the contours of the actuator 208. The arms 402 are resilient in the radial direction, but less resilient (i.e., stiffer) in the axial direction.

The torque transfer element 202 and interior of the actuator 208 are sized such that the distal portions 405 and/or the distal protrusions 406 of the arms 402 abut with the contours of the inner wall of the actuator 208.

The distal protrusion 406 may include curved or angled portions, and/or chamfered or filleted edges. The leading side of the distal protrusion 406 may be inclined with respect to a radial direction. In some examples, the leading side of the distal protrusion 406 may be inclined to complement the rear stop 909 of the actuator 208, as best shown in FIG. 10.

At least a portion of each arm 402 is formed of resilient material, such that it may deform radially inwardly in response to external forces and resiliently return to, or towards, its resting state when the force is removed. Each arm 402 may be constructed to resist deformation in an axial direction.

The torque transfer element includes three substantially identical arms 402 positioned equidistantly around the perimeter of the central body 401. However, the torque transfer element may include any number of arms 402, for example 1, 2, 3, 4, 5, or 6 arms. Each arm 402 of the torque transfer element 202 is configured to rest on, or otherwise interact with, a corresponding ramp 905. Accordingly, in examples comprising three arms, the actuator includes three corresponding ramps, front stops, and rear stops. The position of the ramps on the interior of the actuator corresponds to the position of the arms of the torque transfer element. In other examples, there may be a dissimilar number of arms 402 and contours. In one example, there may be three arms 402 and five contours.

Although not shown, the position of the contours and arms may be reversed on the actuator 208 and torque transfer element 202. For example, the actuator 208 may include one or more inwardly projecting arms. The arms may be substantially as described herein, but attached at their proximal end to an inner wall of the actuator 208 and including their distal end or distal protrusions extending into an inner part of the interior. In this example, the torque transfer element includes contours positioned on its central body, the contours including a ramp, and optionally a front stop and rear stop at front and rear ends thereof.

### Spring/Biasing Member

Axial movement of the torque transfer element 202 in the actuator interior is in part controlled by the axial biasing of the torque transfer element 202 rearwardly (towards the rear wall of the actuator 208), that is, axial biasing of the torque transfer element 202 towards the rear end of the ramp 905. Axial biasing can be produced with springs or the use of other resilient members that exert an axial urging force on the torque transfer element 202.

With reference to FIG. 2, biasing member 207 is positioned in the interior of the actuator 208 between the covering cap 204 and the torque transfer element 202 to exert a rearward biasing force on the torque transfer element 202. However, biasing member 207 may be positioned in other parts of the assembly 201 and still provide the biasing force. For example, the biasing member may be located in the housing 205.

The biasing member 207 urges the torque transfer element 202 towards the rear end 907 of the interior, such that arms 402 are biased towards contact with the surface of the ramp 905 or rear wall 1101. Relative rotation of the actuator 208 and engagement between the distal portion 405 and the ramp 905 causes the movement of the torque transfer element 202 in the axial direction, and relative rotational movement along the surface of the ramp 905. As the biasing member 207 urges the torque transfer element 202 rearwardly, forwards axial movement is resisted by the biasing member 207 and overcome by a countering forward axial force exerted as the actuator 208 rotates the ramp 905 against the torque transfer element 202 in an untightening direction.

The biasing member 207 further biases the first locking element towards the second locking element, as will be described in more detail below.

Where biasing member 207 is a helical compression spring, it is compressed between the covering cap 204 and torque transfer element 202. In other examples, the biasing member 207 may comprise a helical tension spring. Whilst a helical spring is shown in FIG. 2, other suitable springs or biasing elements may be used.

### Rotatable Member

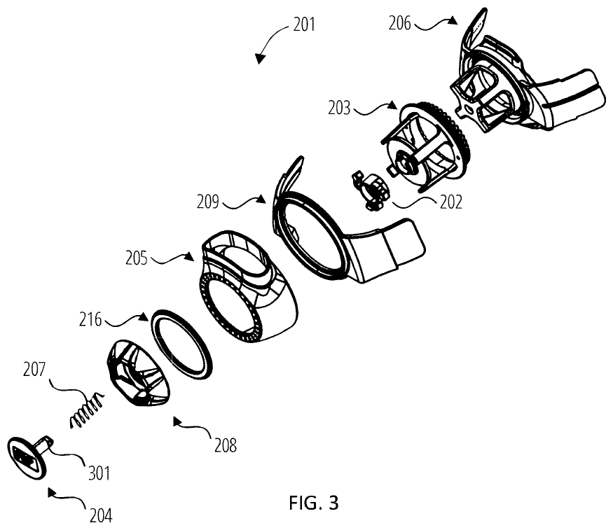

As shown in FIG. 2 and FIG. 3, the adjustment assembly 201 includes a rotatable member 203 for winding, storing, and unwinding the tension element. The rotatable member 203 is connected to a rear end of the torque transfer element 202.

With particular reference to FIG. 5, the rotatable member 203 includes a front body 503 and a rear body 504 being rigidly connected or integrally formed with each other and separated by one or more posts 505. The space between the posts 505 defines windows defining a portion of a gaseous flow path.

The front body 503 of the rotatable member 203 is connected to the torque transfer element 202. The connection may be a rigid connection, or the rotatable member 203 and torque transfer element 202 may be integrally formed. For example, the rotatable member 203 may connect to the torque transfer element 202 via a snap fit connection. The snap fit connection is configured to transfer torque directly from the torque transfer element 202 to the rotatable member 203 at least up to maximum torque levels borne by the torque transfer element 202. The rotatable member 203 and the torque transfer element 202 may be keyed to ameliorate or prevent relative rotation between the components.

The rotatable member 203 includes an annular channel 502 on an outer wall thereof into which the tension element 217 may be wound, stored and/or unwound. In some examples, the rotatable member 203 may have two or more annular channels 502. Each annular channel 502 may be configured to receive one or more tension elements 217. In some examples, two or more annular channels 502 may have a different diameter. Rotation of the rotatable member 203 in a first direction winds the tension element 217 about the annular channel 502 and rotation of the rotatable member 203 in a second direction (opposite the first direction) unwinds the tension element 217 from the annular channel 502. In this way, the length of the tension element that is outside the housing 205 can be shortened or extended by rotation of the rotatable member 203.

The annular channel 502 is positioned on the rear body 504 and is defined by a rim 506 positioned on a front side of the annular channel 502 configured to rotate against or near the inner wall of the housing 205, and a flange 507 positioned on a rear side of the annular channel 502.

The rear body 504 of the rotatable member 203 includes an opening 508 defining a portion of the gases flow path. The opening 508 is surrounded by the annular channel 502.

A first locking element 501 is positioned on the rear side of the flange 507 of the rear end 907 of the rotatable member 203. The purpose of the first locking element 501 is to control the rotation of the rotatable member 203 by reversibly engaging with the base 206, and in particular by interacting with a complementary second locking element 601 mounted on the base 206. The first locking element 501 includes a plurality of rearwardly projecting teeth 509 circularly arranged around the perimeter of the flange 507. The rearwardly projecting teeth 509 may be equally spaced and sized.

The plurality of rearwardly projecting teeth 509 are configured to intermesh with a plurality of forward projecting teeth 602 on the second locking element 601. Each set of teeth are complementarily shaped such that they can intermesh with each other, with the tips of each tooth on one locking element engaging with the bottom of a groove between teeth on the other locking element.

The teeth may be uniformly slanted off-center to impart a directional bias to the first locking element 601, such that the rearwardly projecting teeth 509 may be rotated against the second locking element 601 with less resistance in one direction compared to the opposite direction. Varying the angle of the off-center slant affects the resistance to rotation. In one example, rearwardly projecting teeth 509 are angled at about 30 degrees and have a depth of about 0.4 mm. The angle of the off-center slant on the teeth allows the rotation of the rotatable member 203 in the tightening direction, but, in concert with the rearwards biasing force, inhibits the rotation of the rotatable member in the untightening direction.

Relative rotational movement of the rotatable member 203 and the base 206, and more specifically the first locking element 501 and the second locking element 601 thereof, may provide for indexed rotation of the actuator 208. That is, the rearwardly projecting teeth 509 and the forward projecting teeth 602 create a plurality of detents whereby the rotatable member 203 may rotate with respect to the base 206 in a plurality of discrete increments. For example, the adjustment assembly 201 may comprise about 20-60 detents, preferably about 30-50 detents, and most preferably about 40 detents (i.e., for a full 360° rotation of the rotatable member 203). This may be advantageous for enabling precise and/or repeatable adjustment of the headgear. In other examples, the rotatable member 203 may be configured to rotate substantially continuously rather than incrementally.

The biasing member 207 operates to bias the torque transfer element 202 against the ramp 905, and may further operate to bias the first locking element 501 against the second locking element 601. The torque transfer element 903 is biased towards the rear end of the ramp 905 and/or towards the rear wall 1101 of the actuator. Rotation in the untightening direction causes the ramp 905 to act on the arms 402 of the torque transfer element 903, inducing an axial force opposing the biasing force and causing the arms 402 to slide forwards along the ramp 905 towards the front stop 908.

The biasing member 207 exerts a biasing force on the rotatable member 203, and the first locking element 501, towards the second locking element 601. Accordingly the first locking element 501 is urged against the second locking element 601. The biasing force must be overcome for the first locking element 501 to be separated from the second locking element 601.

The cooperation of the first locking element 501 and the second locking element 601 allows the incremental tightening of the tension elements 217 and acts as a ratchet on the tensioning element. Engagement of the first locking element 501 with the second locking element 502 allows the rotation of the rotatable member 203 in a tightening direction for the tensioning element as the respective teeth slip over each other, but prevents or inhibits the rotation of the rotatable member 203 in the untightening direction for the tensioning element.

The biasing force is overcome by screw action of the ramp 905 on the torque transfer element 202. Rotation of the actuator 208 in the untightening direction causes the distal portions of the arms 402 to slide along the ramp 905 such that the torque transfer element 202 is translated axially forwards in the interior of the actuator 208. The rotational force overcomes the rearwards biasing force exerted by the biasing member 207 against the torque transfer element 202 and moves the rotatable member 203 forwards, disengaging the first locking element 501 from the second locking element 601 to permit rotation of the rotatable member 203 in the untightening direction.

During cooperation of the first locking element 501 and the second locking element 601, the action of the teeth of the first locking element 501 slipping over teeth of the second locking element 601 into contiguous grooves in the tightening direction may produce a clicking noise that gives the user audible feedback on the tightening of the tension elements. The incremental movement of the first locking element 501 relative to the second locking element 601 may also produce tactile feedback, as the user feels each incremental shift of the first locking element 501 as a vibration in the actuator and/or face-contacting portions of the adjustment assembly 201.

### Housing

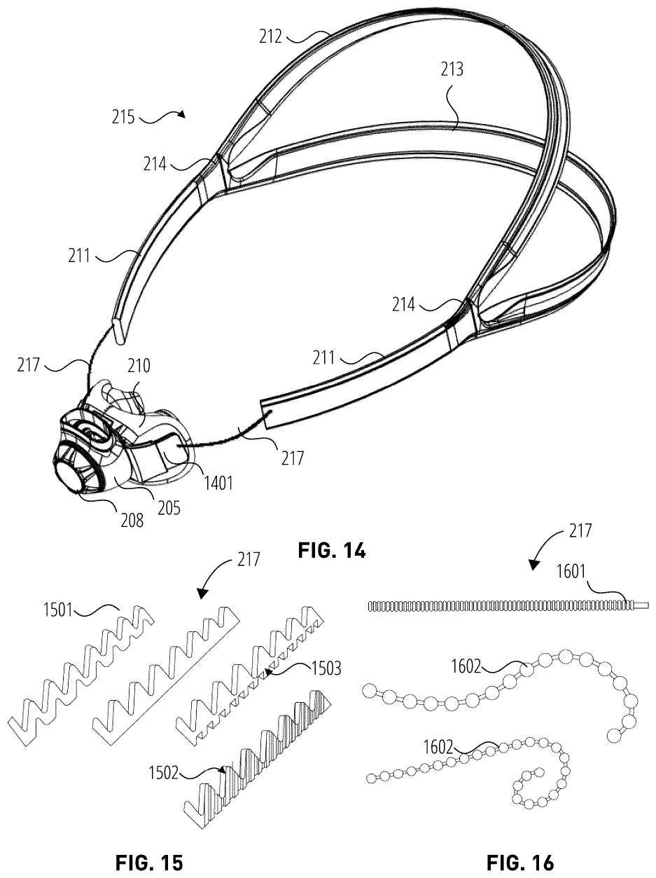

As shown in FIG. 2, FIG. 3, FIG. 13 and FIG. 14, a housing 205 is provided for retaining the rotatable member 203 and stored tension elements 217.

With particular reference to FIG. 13, the housing 205 includes a side wall 1301 that at least partially defines a housing interior for retaining the rotatable member 203. A front end of the housing 205 includes a front opening 904 facing the actuator 208 and a rear end of the side wall includes a rear opening facing the base 206.

The side wall 1301 of the housing 205 includes a breathing gases inlet 1302 surrounded by a connection member 1303 extending outwardly from the periphery of the breathing gases inlet 1302 and configured to connect with a breathing gases tube (not shown). The breathing gases inlet 1302 may be located at any position on the side wall 1301 of the housing 205, and the desired location may depend on a user's preference of the orientation of a breathing gases tube. For example, the breathing gases inlet 1302 may be positioned on an upper side of the side wall 1301, where the breathing gases tube is configured to traverse over the user's nasal bridge. In other examples, the breathing gases inlet 1302 may be provided at a front of the housing. In yet other examples, the housing 205 may comprise two or more breathing gases inlets 1302 which may be selectively coupled with a breathing gases tube or plugged at the user's preference. In any of these examples, the breathing gases inlet 1302 may be configured to connect to the breathing gases tube directly or via an elbow. The patient interface may comprise a swivel joint or a ball joint between the breathing gases inlet 1302 and the elbow.

The front end of the housing 205 is configured to form a rotatable seal with the actuator 208, such that the actuator 208 may rotate relative to the housing 205. To better achieve a rotatable seal, an intervening seal ring (e.g., an O-ring 216) may be positioned between a peripheral edge of the rear end of the actuator 208 and the front end of the housing 205. In some examples, the seal may be overmolded to the housing 205 and/or the actuator 208. In some examples, the seal may comprise a sealing lip configured to provide a pressure-assisted seal, in use.

The rear end of the housing 205 connects to a face plate 209, described in further detail below, such that the rear opening of the housing 205 communicates with an opening on the face plate 209 and the central opening on the base 206.

The housing 205 may be configured to be rotatable against the face plate 209 and/or base 206, and rotatable with respect to the internal components such as the rotatable member 203 and first and second locking elements. Thus, the housing 205 may allow the rotatability of the breathing gases inlet 1302, and associated breathing gases tube, relative to the base 206. Alternatively, the housing 205 may be rigidly connected to the base 206 such that the housing 205 does not rotate relative to the base 206, and the breathing gases inlet 1302 and associated breathing gases tube are held in a fixed position relative to the base 206.

### Face Plate

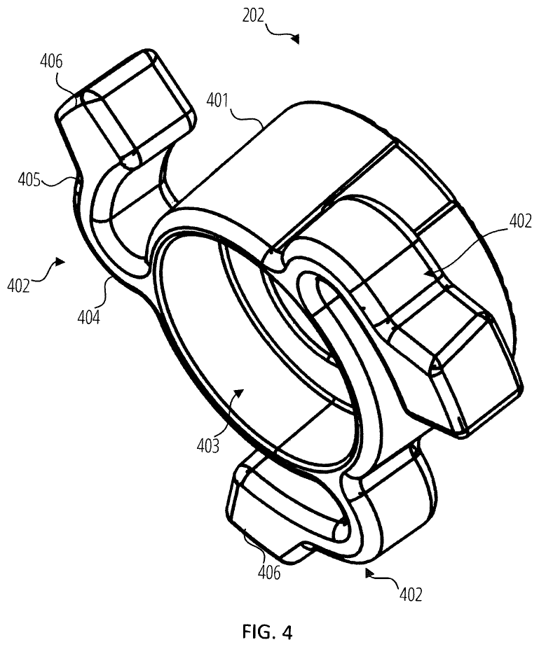

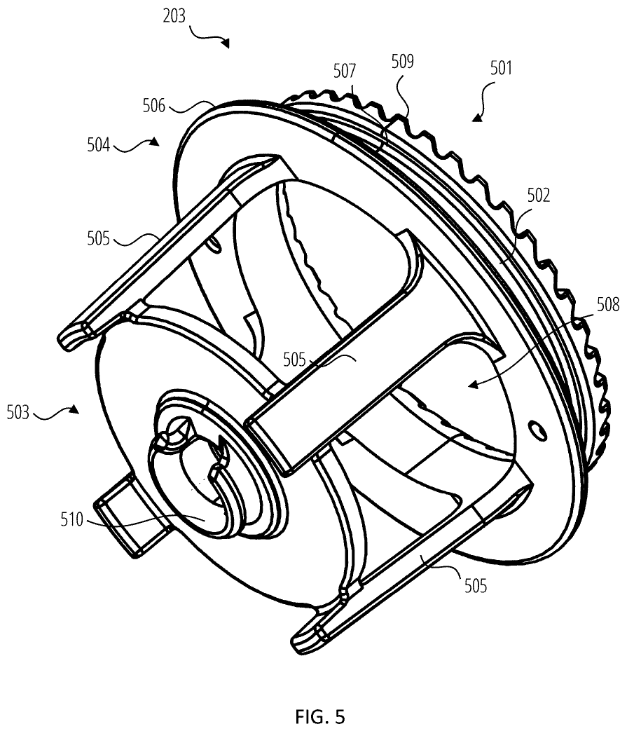

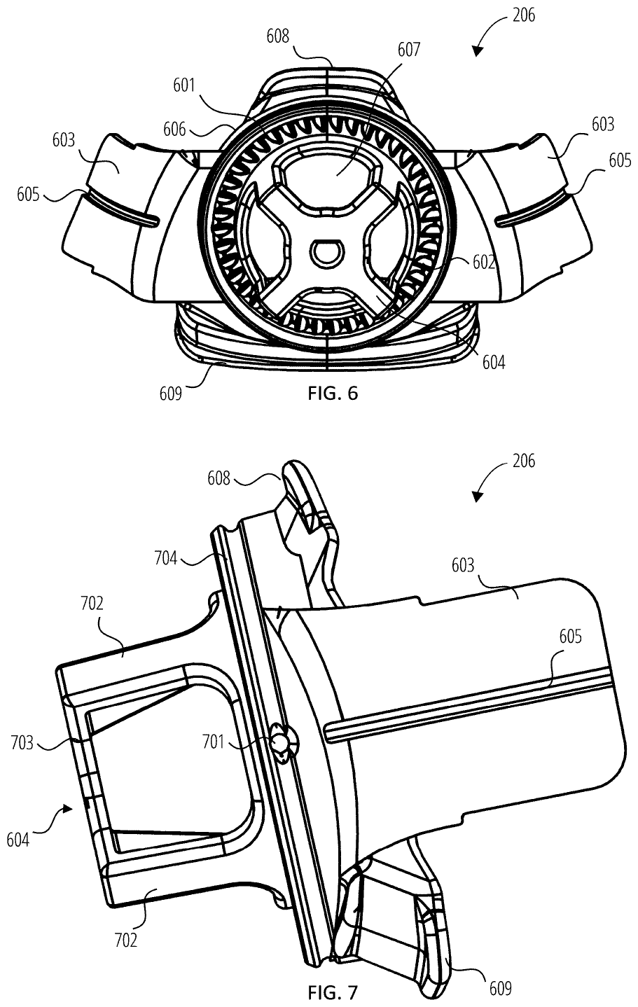

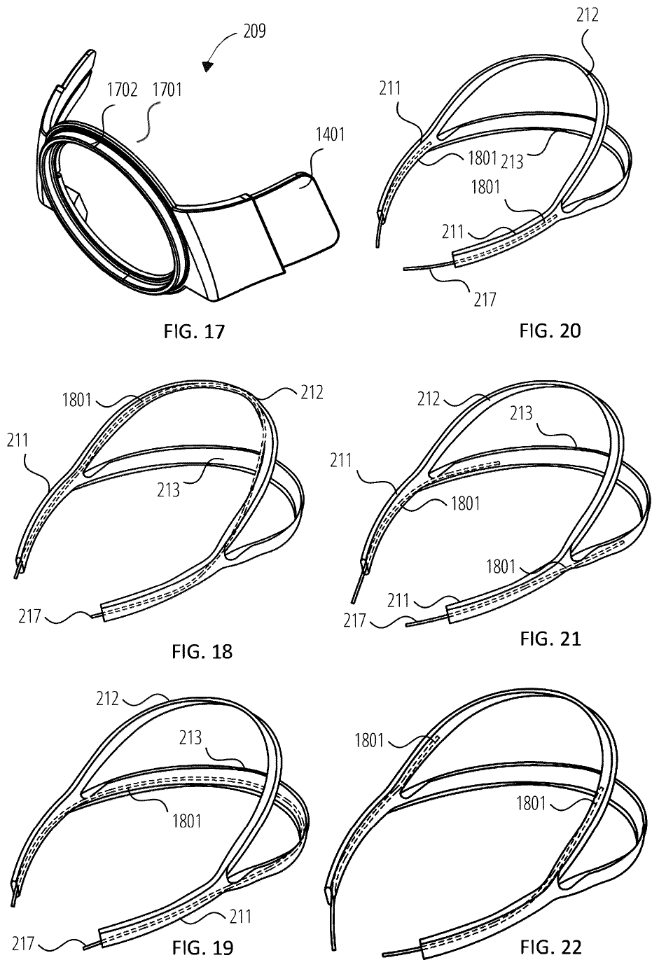

Referring to FIG. 17, the assembly includes a face plate 209 including a middle section 1701 and left and right side arm covers 1401 (see FIG. 7). The face plate 209 sits between the base 206 and the housing 205, and includes a raised ridge 1702 for sealingly engaging with the inside edges of the rear opening of the housing 205. The left and right side arm covers 1401 extend outwardly and rearwardly from the left and right sides of the face plate 209 and together with the corresponding left and right side arms 603 on the base 206, form covered side channels including a path for the tension element 217 to pass from the adjustment assembly to the headgear.

### Base

Referring to FIG. 2, and FIG. 3 the adjustment assembly 201 includes a base 206 for connecting the assembly to a face seal 210, and for directing the one or more tension elements to the headgear. The base 206 is configured to be held in a fixed position on the face of the user by the headgear in use, via headgear straps and/or one or more tension elements 217. The base 206 in turn anchors the seal 210 in a fixed position. As such, the actuator 208, torque transfer element 202 and rotatable member 203 may rotate in relation to the base 401.

The base 206 is positioned to the rear of the rotatable member 203 and housing 205. With particular reference to FIG. 6 and FIG. 7, the base 206 includes left and right side arms 603, a second locking element 601, a forwardly projecting mount 604, and one or more parts for connecting to a seal 210 (such as top flange 608 and bottom flange 609).

The left and right side arms 603 extend outwardly and rearwardly from left and right sides of the base 206. Each side arm includes a guide channel 605 for directing the tension elements 217 to the headgear. The guide channels 605 retain the tension element 217 along a path from a point where the tension element 217 projects from the rotatable member 203 towards the side arms of a headgear 215. The guide channel 605 may include recessed portions within the side arms that are configured to receive the tension element 217 along at least a portion of the path. Alternatively, or additionally, the guide channel 605 may include raised sections such as protrusions, lugs, or ridges to define the path and guide the tension element 217.

The base 206 includes a main body 606 including a central opening 607 surrounded by the second locking element 601. The second locking element 601 is shaped and sized to complementarily match the shape and size of the first locking element 501. As such, the second locking element 601 includes a plurality of forwardly projecting teeth arranged on a front side of the main body 606, as described above. Each of the plurality of teeth are circularly arranged around the perimeter of the opening 607, are equally spaced and sized, and are uniformly slanted off-center. The angle of the off-center slant is reciprocal/inverse to the angle of the off-center slant of the teeth in the first locking element 501, such that the first and second locking elements can reversibly intermesh. A rim 704 is located around the outer perimeter of the second locking element 601, the rim 704 configured to engage with the rear of the face plate 209 at or about the middle section 1701. The rim 704 may include one or more tension element apertures 701 for the tension element 217 to slidably pass towards the guide channels 605.

The first locking element 501 is biased towards the second locking element 601 on the base 206, for example by biasing member 207, such that the teeth of the first locking element 501 intermesh with the teeth of the second locking element 601. Rotational force in the tightening direction induces sufficient axial/forwards force to overcome the biasing force and causes the teeth of the first locking element 501 to shift forwards and incrementally slide over the teeth of the second locking element 601. This may be achieved by setting the angle at which the teeth of the first locking element 501 and second locking element 601 contact each other, relative to the direction of the tightening rotational force, such that the resisting forces to the rotational force (e.g., frictional forces) may be overcome by a user rotating the actuator with their fingers.

The first and second locking elements may be configured such that rotational forces in the untightening direction produces relatively less axial forwards force to overcome the biasing force and therefore the teeth of the first locking element 501 do not slide over the teeth of the second locking element 601 in the other direction. This may be achieved by setting the angle at which the teeth of the first locking element 501 and second locking element 601 contact each other, relative to the direction of the untightening rotational force, such that the resisting forces to the rotational force (e.g., frictional forces) are not overcome by a user rotating the actuator with their fingers. That is, the teeth may have an asymmetric profile, such as a sawtooth profile. Accordingly, the first locking element 501 must be axially disengaged from the second locking element 601 (e.g., by rotation of the torque transfer element 202 in the untightening direction to shift the rotatable member 203 forwards) in order to allow the rotatable member 203 to rotate in the untightening direction.

In an alternative example, the first and second locking elements may be configured such that rotational forces in the untightening direction induce similar axial forwards force to overcome the biasing force, allowing the teeth of the first locking element 501 to incrementally slide over the teeth of the second locking element 601. In this example, rotation of the actuator 208 in the untightening direction may allow the incremental untightening rotation of the rotatable member 203.

The base 206 includes a mount 604 including one or more mount posts 702 and a forward projection 703 extending from the main body 606. Mount 604 is configured to nest within the rotatable member 203, and particularly in the space defined between the opening 508, posts 505, and front body 503. The rotatable member 203 is thus rotatable about mount 604. The forward projection 703 has an opening 607 configured to receive the connector 1201 of the pin 301 of the covering cap 204 (described in further detail below), and in particular to receive a snap fit connector from the pin 301. The posts 505 are spaced apart around the opening 607, and gas flow windows are defined by the spaces between the mount posts 702, along with the forward projection 703 and main body 606.

The base 206 includes one or more parts for connecting to a seal 210 which is configured to contact the user's face and provides breathing gases to the user's nose and/or mouth in the provision of respiratory therapy. At the rear end of the base 206, top flange 608 and bottom flange 609 engage with the seal 210, such that an opening of the seal 210 may envelope around and sealingly engage the top and bottom flanges and outer sides of the base 206.

### Seal

The adjustment assembly 201 may be configured to communicate with a patient interface, such as a cushion or seal module. Referring to FIG. 2 and FIG. 14, a nasal pillows-type seal is shown. The seal 210 includes a front opening including a peripheral edge configured to surround the top and bottom flanges of the base 206 and form an airtight seal against a wall of the base 206. A portion of the base, including the top and bottom flanges, projects into the interior cavity of the seal 210. The seal 210 comprises a pair of nozzles each configured to form a seal about a respective one of the user's nares for the supply of pressurized gases to the user's nasal passages in use. In other examples, the seal may include a nasal cannula (contacting or extending into the nares), nasal mask (sealing around the nose), compact nasal mask (sealing around the nares and/or a lower part of the nose), nasal pillows (sealing around or on an inner side of each of the nares), oral mask (sealing around the mouth), full face mask (sealing around the nose and mouth), compact full face mask (combining an oral mask and a nasal mask or nasal pillows), a total face mask (sealing around the eyes, nose and mouth) endotracheal tube (inserted into the trachea through the mouth or nose), tracheostomy tube (inserted into the trachea through an incision in the neck), or other known types of interfaces.

### Covering Cap

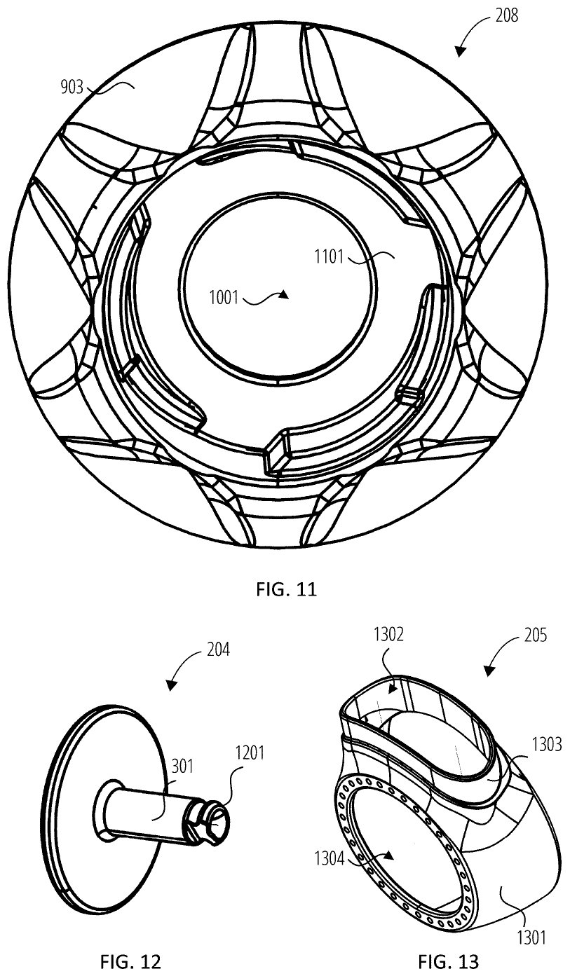

The front of the actuator 208 includes a front opening 904 and an angled circular front edge configured to engage with a circular edge of a covering cap 204, illustrated in FIG. 2, FIG. 3, and FIG. 12. The covering cap 204 includes a substantially circular disc including an outer surface that may be the foremost point of the adjustment assembly 201 when worn by a user, and it may bear branding or other information. The covering cap 204 is configured to engage with the actuator 208 to seal the front opening 904. The covering cap 204 may engage the front end of the actuator 208 in an airtight seal. The covering cap 204 includes a pin 301 extending axially from the center of the covering cap 204. The pin 301 has a distal end including a connector 1201 for engaging with a mounting portion positioned within the housing 205 or base 206. In particular, the connector 1201 is configured to engage with the mount 604 on the base 206. The connector 1201 may be a snap fit connector configured to engage with a receiving portion (e.g., a snap fit receiver) in the base 206. The connector 1201 may include a distal flange portion that inhibits the detachment of the pin 301 from the base 206 and a narrowed portion that is encircled by the receiving portion on the base 206. The narrowed portion is of smaller diameter relative to that of the central pin. Accordingly, the pin 301 is configured to project through the interior of the actuator 208. Pin 301 passes through the central opening of the torque transfer element 202 and rotatable member 203. Accordingly, the torque transfer element 202 and rotatable member 203 can freely rotate about the pin 301. In one example, the pin 301 is of sufficiently smooth material to allow a low friction interaction between the pin 301 and the torque transfer element 202 and rotatable member 203. In another example, the pin 301 is of a sufficiently narrow diameter such that it does not interact with the inner sides of the apertures of the torque transfer element 202 and rotatable member 203. The covering cap 204 may be rigidly connected to the base 206, or it may be rotatably connected to the base 206.

The connection between the covering cap 204 and the base 206 may assist or be wholly responsible for retaining the intervening components in place (including the actuator 208, torque transfer element 202, rotatable member 203, and housing 205).

### Gases Flow Path

The adjustment assembly 201 includes a gases flow path between the breathing gases inlet 1302 and the seal 210. The gases flow path may be substantially airtight. The breathing gases flow path enters the assembly at the breathing gases inlet 1302, and passes through the windows and central aperture of the rotatable member 203, the windows of the mount 604 and aperture of the base 206. The gases flow path is in contact with the torque transfer element 202 and rotatable member 203.

### Tension Element

The tension element 217 is for transferring the tensioning forces from the rotatable member 203 onto the headgear. One or more tension elements 217 are configured to engage with the rotatable member 203 so that they may be wound, stored, and unwound from the rotatable member 203, and the annular channel 502 in particular. The tension element 217 may be a substantially inextensible or inelastic material that is suitable to transfer such tensioning forces. The tensioning element has a tensile strength sufficient to remain substantially inextensible or inelastic at tensions associated with blow-off and tube-drag forces. Alternatively, at least a portion of the tension element 217 may be elastic or extensible.

The tension element 217 may be of a relatively narrow gauge or diameter, such that storage of a spool of the tensioning element is possible in a relatively small volume, such as the volume around the annular channel 502 and housing 205. The tension element 217 may therefore be in the form of a thin elongate filament. The tension element 217 may include a strand of flexible material, two or more strands of material braided, woven, or otherwise formed together into a line, thread, ribbon, or tape.

The tension element 217 may include features to increase grip or purchase with the headgear and/or rotatable member 203. For example, FIG. 16 shows several alternative examples of the tension element, including a flexible filament including a plurality of ribs 1601 spaced along its length, or a flexible filament including a plurality of beads 1602 spaced along its length (i.e., a ball chain or snake link). The tension element may include a length of chain links, corrugations, notches, ribs along its length.

The tension element may be configured to have rigidity in one dimension and flexibility in an orthogonal direction. In this example, the tension element may be in the form of a ribbon or tape. FIG. 15 shows alternative examples of corrugated ribbons 1501 including greater flexibility in one dimension compared to an orthogonal dimension. In some configurations, relief recesses 1503 can be provided to increase the flexibility of the corrugated ribbons 1501. In an example, the corrugated ribbons 1501 may include a series of grooves 1502 positioned along one side thereof.

One or more tension elements are configured to connect to the rotatable member 203 at one or more end portion thereof. In an example, the tension elements 217 connect at a position on the rotatable member 203, for example on or near the annular channel 502. Rotation of the rotatable member 203 in a first direction or a second direction drives the tension elements 217 in a corresponding first or second direction. For example, each tension element 217 is configured to wind or unwind about the rotatable member 203 as it is rotated.

Alternatively, one or more tension elements may connect to the rotatable member 203 at an intermediate point along its length, such as a mid-point. For example, a single continuous tension element may extend down a headgear strap on one side of the patient interface assembly, connect with the rotatable member 203, then extend up another headgear strap on an opposing side of the patient interface assembly. In another example, a single continuous tension element may extend down an upper side strap of the headgear on one side of the patient interface assembly, connect with the rotatable member 203, then extend up a lower side strap on the same side of the patient interface assembly (with a second tension element doing the same on the opposing side of the patient interface assembly).

### Headgear

The adjustment assembly 201 may be connected to headgear, for example via one or more tension elements. The headgear may be any type of headgear used in respiratory therapy. Preferably, the headgear includes flexible elements that may be tightened about a user's head and face. The headgear may also include rigid elements, for example to hold the shape of the headgear to improve the ease of donning and doffing, and to better distribute tightening forces around the headgear.

The headgear may include one or more side straps 211, a top strap 212 and a back strap 213, and one or more pathways 1801 for receiving the tension element 217. Each of the one or more side straps 211 connect at a distal end thereof to a junction portion 214. The top strap 212 connects to left and right side junction portions 214 at distal ends thereof. The back strap 213 connects to left and right side junction portions 214 at distal ends thereof.

One or more of the side straps, top strap and back strap may be substantially elastic or have some degree of stretchiness. Alternatively, or additionally, one or more of the side straps, top strap and back strap may be substantially inextensible.

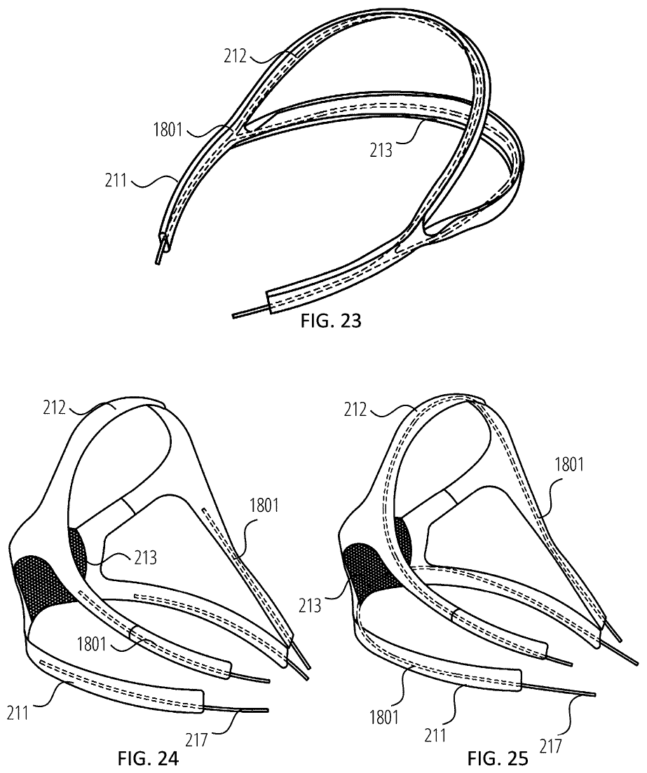

FIG. 20 to FIG. 23 illustrate examples of two-point headgear comprising a pair of side straps 211 configured to attach to respective sides of a patient interface with a two-point attachment. FIG. 24 and FIG. 25 illustrate examples of four-point headgear comprising a pair of upper side straps, configured to respectively pass between an eye and an ear of the user, and a pair of lower side straps, configured to respectively pass beneath an ear of the user.

The rotatable member 203 may advantageously enable simultaneous and symmetric adjustment of the length and/or tension of two or more headgear straps. In other examples, the adjustment assembly 201 may be configured to simultaneously adjust the length and/or tension of two or more headgear straps in different proportions. For example, in a patient interface assembly comprising a full-face mask with a four-point headgear attachment, it may be preferable to increase tension relatively more in the upper straps than the lower straps as the actuator 208 is rotated, to improve sealing in the user's nasal bridge region. In another example, the patient interface may comprise a forehead support providing a pivot point, and it may be desirable to adjust a length of the lower straps relatively more than the upper straps as the actuator 208 is rotated.

In some examples, the adjustment assembly may be configured to provide independent adjustment of the tension in two or more straps. For example, the adjustment assembly may have two actuators each associated with different straps of the headgear.

### Pathways

As shown in FIG. 20 to FIG. 25, the headgear 215 may include one or more pathways 1801 for at least one tension element 217 (pathways 1801 are shown in bold line on the Figures). The one or more pathways 1801 may be positioned on, or in, the one or more side straps 211 of the headgear 215. In addition, the pathways 1801 may be positioned on, or in, the top strap 212. In addition, the one or more pathways 1801 may be positioned on, or in, the back strap 213.

The pathways are positioned along one or more side straps of the headgear. The one or more pathways may be a continuous pathway, such as a continuous pathway positioned along left and right side straps 211 and one or both of the top strap 212 and the back strap 213 as shown in FIG. 18, FIG. 19, FIG. 23 and FIG. 25. Alternatively, the one or more pathways may include separate pathways, for example a left side pathway positioned on a left side of the headgear (e.g., positioned on the left side strap and optionally a portion of one or more of the top strap 212 and back strap 213) and a right side pathway positioned on a right side of the headgear (e.g., positioned on the right side strap and optionally a portion of one or more of the top strap 212 and back strap 213) as shown in FIG. 20 and FIG. 24.

Pathways 1801 may be flexible, semi-rigid or substantially rigid, include a substantially rigid component, or include a thermoformed plastic element. A semi-rigid or rigid pathway may advantageously impart a non-linear shape upon the respective flexible tension element, thereby enabling the tension to be routed around the user's ears even when under tension, for example. The one or more pathways may include a channel or tube positioned on the headgear configured to receive the tension elements therethrough. The one or more channel or tube may be formed of, or include a portion of, flexible, rigid, or semi-rigid material. For example, the channel or tube may be formed of a knitted material, a woven or non-woven textile material, a flexible polymer material. Alternatively, the channel or tube may be formed of, or include a portion of, a more rigid material such as a thermoformed polymer material or a metallic material.

The one or more pathways may be integrally formed with a part of the headgear. For example, the pathways may be formed as part of a portion (e.g., a textile portion or a rigid portion) of the side straps 211, top strap 212, and/or the back strap 213. Alternatively, the one or more pathways may be, or include, separately formed components that are connected to the headgear (e.g., at one or more positions along the headgear). For example, the one or more pathways may include channels that are configured to connect to the headgear via a reversible fastening connection such as a hook-and-loop fastener (e.g., Velcro®), buttons, domes, zips, or the like.

In an example, two separate pathways may be positioned along each of the side straps 211 of the headgear, configured to receive two separate tension elements along each side strap. The number and precise position of the pathways will depend in part on the nature of the patient interface, and in particular whether the patient interface is configured as a nasal pillows, nasal mask or full face mask, for example. For a full-face mask, a four-point configuration for headgear and pathways may be preferred, whereas for nasal and oro-nasal masks, a two-point configuration is sufficient. Examples of four-point configurations for headgear and pathways are shown in FIG. 24 and FIG. 25.

The one or more tension elements 217 are positioned along the one or more pathways in the headgear, in use. The tension element may connect to one or more anchor points on the headgear or pathways, e.g., in the side straps 211 as shown in FIG. 20 and FIG. 24, the back strap 213 as shown in FIG. 21, the top strap 212 as shown in FIG. 22. Alternatively, the tension element may be configured to run through the pathways and connect at each end thereof to the adjustment assembly 201, as shown in FIG. 18, FIG. 19, FIG. 23, and FIG. 25. In other examples, the headgear may comprise combinations of terminated and looped tension elements. In some examples, a tension element may bifurcate. As shown in FIG. 23, the tension element may include a bifurcated middle section in which the middle portion of the tension element includes two separate filaments that can follow a top strap 212 and a back strap 213, respectively, and converge towards the ends thereof to a single filament to follow each side strap.

### Engaged/Disengaged Configuration

The torque transfer element 202 is translatable along its axis of rotation between an engaged configuration in which the first locking element 501 and the second locking element 601 are in rotational communication, and a disengaged configuration in which the first locking element 501 and the second locking element 601 are not in rotational communication.

The engaged configuration and disengaged configuration are defined by the relative position of the first locking element 501 and the second locking element 601, and the relative position of the arms 402 of the torque transfer element 202 on the ramp 905.

In the engaged configuration, the distal portion of the arms 402 of the torque transfer element 202 are aligned or in contact with the rear end of the ramp 905, the first locking element 501 is in contact with, and urged towards, the second locking element 601 due to the biasing force exerted by the biasing member 207. In the engaged configuration, the rotatable member 203 may be rotated in a tightening direction so as to incrementally wind in the tensioning element about the annular channel 502. Restoring forces exerted by the tensioning elements on the rotatable member 203 are resisted due to the intermeshing of the slanted teeth on the first locking element 501 and second locking element 601.

In the disengaged configuration, the distal portion of the arms 402 of the torque transfer element 202 are aligned or in contact with the front end of the ramp 905 and the first locking element 501 is separated from the second locking element 601 along the direction of the axis of rotation. The first locking element 501 and the second locking element 601 are thus held in a separated or disengaged state in part due to the biasing force being overcome by the resisting force between the front end of the ramp 905 and the distal portion of the arms 402.

In the disengaged configuration, the rotation of the rotatable member 203 is not constrained by a locking element and it may freely rotate in either the tightening or untightening direction. Accordingly, in the disengaged configuration, the tension elements may be unwound from the rotatable member 203. The user may unwind the tension elements by rotating the actuator 208 in the untightening direction, or alternatively the user may pull directly on the tension elements or headgear straps attached thereto so as to directly unwind the tension elements.

Interaction between the torque transfer element and the actuator

The torque transfer element 202 and interior of the actuator 208 are sized such that the torque transfer element 202 is able to move along its axis of rotation within the interior. This allows the axial movement of the torque transfer element 202 within the interior as it runs along the ramp 905.

Axial and rotational movement of the torque transfer element 202 is produced by the interaction between the distal protrusion 406 and the contours of the interior of the actuator 208. The distal protrusions 406 are configured to run along the surface of the ramp 905, and to abut against the front stop 908 and rear stop 909. In this way, the rotation of the actuator 208 is communicated to the torque transfer element 202 via the contours of the actuator 208 and in particular the front stop 908 and rear stop 909.

The inwardly protruding contours (e.g., the ramp 905, front stop 908 and rear stop 909) are configured to interact with the torque transfer element 202 in different ways depending on the direction of rotation of the actuator 208. The contours are configured such that rotation of the actuator 208 in a first direction (a tightening direction) causes the engagement of the leading side of the distal protrusion 406 of the torque transfer element 202 with the rear stop 909. Rotation of the actuator 208 in a second direction (untightening direction) causes the engagement of the distal protrusion 406 and/or distal portion 405 first with the ramp 905, then optionally with the front stop 908.

As noted above, the torque transfer element 202 is biased towards the rear end of the interior of the actuator 208, and the arm 402 sits against the rear wall 1101 and/or the rear stop 909. When a user rotates the actuator in a tightening direction, the rear stop 909 abuts against the distal portion 405 of the torque transfer element 202, which brings the actuator 208 into rotational communication with the torque transfer element 202 such that torque is transferred from the actuator 208 to the torque transfer element 202. Torque is transferred from the torque transfer element 202 to the rotatable member 203, which transfers the torque to the tension element connected thereto which in turn applies tightening force to the headgear. As the headgear is tightened against the head of the user, the amount of torque required to further tighten the headgear increases. The torque transfer element 202 is configured to slip over the contours of the actuator when the tightening force applied by the user reaches a threshold. In this way, the assembly may be configured to prevent overtightening of the headgear by limiting the amount of torque a user can apply to the tension elements via the actuator 208.

When the actuator 208 is rotated in an untightening direction, the arm 402 is urged forwards along the ramp 905 such that the torque transfer element 202 is translated axially forwards, e.g., towards the covering cap 204 of the actuator 208. Thus, rotation in the untightening direction retracts the torque transfer element into the interior of the actuator. Optionally, the arm 402 may move along the ramp 905 until it abuts the front stop 908, although it may not be necessary for the torque transfer element 202 to move all the way along the ramp 905 in order to disengage the first locking element 501 from the second locking element 601. Axial movement of the torque transfer element 202 is transferred directly to the rotatable member 203 and the first locking element 501, thus causing a disengagement of the first locking element 501 from the second locking element 601. Once disengaged, the rotatable member 203 may freely rotate in the untightening direction. The headgear may therefore be loosened by further rotating the actuator 208 in the untightening direction, or by applying tension to the headgear to pull the tension elements from the rotatable member 203.

### Preventing Overtightening—Torque Threshold and Predetermined Spring Constant

The adjustment assembly 201 is in operational communication with the headgear via one or more tension elements 217, which are configured to apply and release tightening forces from the rotatable member 203 to the headgear straps. When a user rotates the actuator 208 in a tightening direction, tightening force is transferred to the rotatable member 203 which drives the tension elements 217 in a tightening direction. The effective length of the tension elements 217 is shortened, thus pulling the headgear and headgear adjustment assembly closer together, tightening the headgear about a user's head, and increasing the force pushing the seal onto the user's face.

To avoid overtightening of the headgear, the torque transfer element 202 is configured to deform against the actuator 208 when the torque applied to the actuator by the user reaches a threshold level. More specifically, the arms 402 of the torque transfer element 202 are configured to deform radially inwardly over the actuator contours such that the actuator 208 rotates independently of the torque transfer element 202 above a set torque level. The deformation of the arms 402 may be in any part of the arm, or may be more dominant in the proximal portion 404 or distal portion 405.

The threshold torque may be predetermined to be substantially equivalent to the torque required to achieve the recommended force required to hold the seal 210 onto the face of the user and avoid blow-off. For example, the threshold torque may be the torque at or near the level required to balance forces that in use act to move the seal away from the user's face, including typical blow-off forces created by the flow generator during CPAP therapy as well as tube drag forces.

The assembly is therefore configured such that the torque transfer element 202 will sufficiently deform at the torque threshold. This may be achieved by modulation of properties of the torque transfer element 202 and/or the actuator 208.

The number of arms 402 on the torque transfer element 202 may be modulated to achieve a particular torque threshold. Alternatively, or additionally, each arm 402 may be constructed to have a predetermined spring constant, which may be achieved by use of materials including known strength and resiliency properties and/or manufacturing processes known to produce an arm including a particular spring constant. The predetermined spring constant is proportional to the amount of torque required to deform the arm, and therefore proportional to the torque threshold.

The torque required to deform the arm 402 about the contours may be adjusted by modulating the respective shape and size of the abutment faces on the rear stop 909 and the distal protrusions 406. Variations may be made to the depth of contact between the abutment faces or the relative angle of the abutment faces to tune the torque threshold. For example, the depth of contact that the abutment faces of the rear stop 909 and the distal protrusion 406 overlap may be between 3.5 millimeters (mm) and 7.5 mm. Where the abutment faces on the rear stop 909 and distal protrusion 406 are at, or near, right angles to the direction of rotation a greater torque will be required for the distal protrusion 406 to deform and slip over the rear stop 909. Therefore, the abutment faces of the rear stop 909 and distal protrusion 406 may angle or slope away from right angles to the direction of rotation. The angle may be between 10 and 45 degrees, for example.

The actuator 208 and the torque transfer element 202 in combination may be said to form a ratchet mechanism. The arms 221 act as pawls and the contours of the actuator 208 act as teeth. The ratchet mechanism permits some relative rotation of the actuator 208 in the first (tightening) direction with respect to the torque transfer element 202 (i.e., when the torque exceeds a threshold), while inhibiting relative rotation of the actuator in the second direction.

The actuator 208 and the torque transfer element 202 in combination may also be said to form a cam mechanism when the actuator 208 is rotated in the second direction. The ramps 905 act as a cam and the arms 402 act as followers.

The actuator 208, torque transfer element 202, rotatable member 203 and base 206 in combination may be said to form a clutch mechanism. Rotation of the actuator 208 in the first direction, below the torque threshold, causes interference between the arms 402 and the contours which transmits torque from the actuator 208 to the torque transfer element 202, and from the torque transfer element 202 to the rotatable member 203. Rotation of the actuator 208 in the first direction, above the torque threshold, engages the ratchet mechanism which causes the arms 402 to deflect inwardly and pass over the contours of the torque transfer element 202, with relatively little (if any) torque transmitted from the actuator 208 to the torque transfer element 202. Rotation of the actuator 208 in the second direction engages the cam mechanism, moving the torque transfer element 202 and the rotatable member in the axial direction, disengaging the first locking element 501 of the rotatable member 203 from the second locking element 601 of the base 206 and allowing the rotatable member 203 to freewheel. Further rotation of actuator 208 in the second direction engages the ratchet mechanism in the reverse direction, causing interference between the arms 402 and the contours which transmits torque from the actuator 208 to the torque transfer element 202, and from the torque transfer element 202 to the rotatable member 203.

### SECOND EXAMPLE

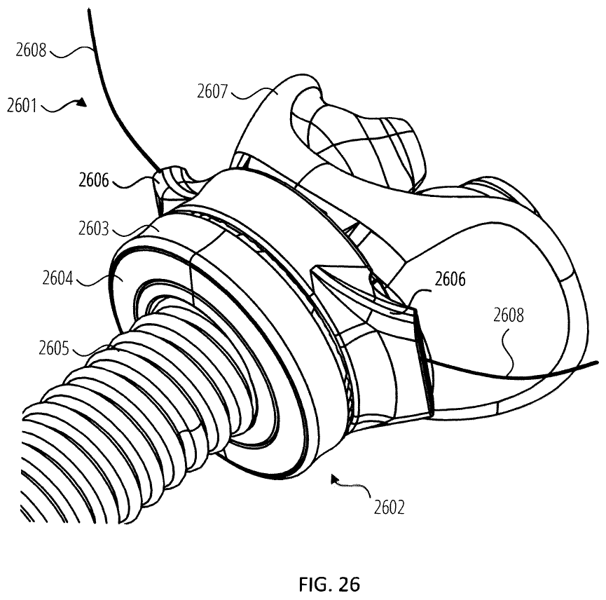

With reference to FIG. 26 to FIG. 34, there is provided a Second Example of an adjustment assembly 2601.

The assembly 2601 may include an actuator 2602, torque transfer element 2701, rotatable member 2702, base 2703, conduit 2704, and housing 2705. Details of each of these components are described below, by way of non-limiting example.

### Actuator

As shown in FIG. 26, the actuator 2602 may be located at a front part of the assembly 2601. Actuator 2602 may include a side wall including an outer surface 2603, an inner wall 3003 with a front end and a rear end, a rear wall 3001. Actuator 2602 may include a central recess defining an interior 3002. The interior 3002 is configured to at least partially house the torque transfer element 2701.

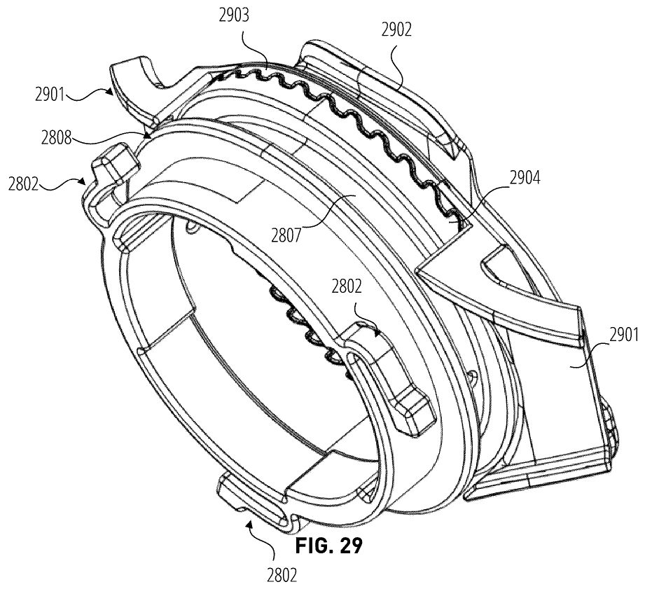

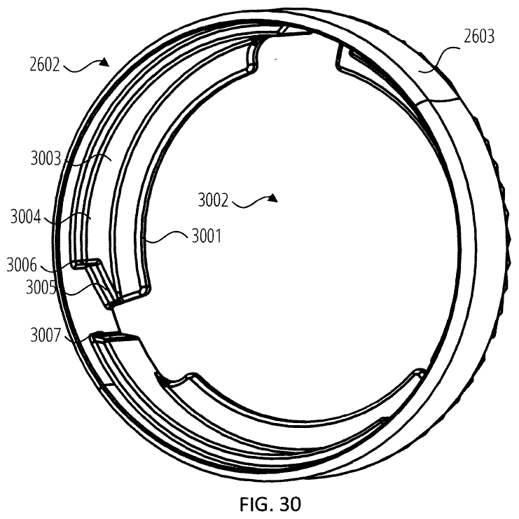

As described above for the First Example (shown in FIG. 2 to FIG. 25), and as shown in FIG. 30, the outer surface 2603 is configured to be actuated by a user, and in particular is configured to be rotated clockwise and counter-clockwise by a user's fingers. The outer surface 2603 includes a generally annular profile, and may include gripping features such as a concave profile along a middle portion of the outer surface 2603, such that the diameter of the outer surface 2603 is reduced towards the middle and greater towards front and rear portions of the outer surface 2603.

The inner wall 3003 includes at least one inwardly protruding contour 3004 configured to interact with the torque transfer element 2701. As with the First Example, a function of the contour 3004 is to translate rotational movement of the actuator 2602 to axial movement in the torque transfer element 2701.

The contour 3004 includes a ramp 3005 along a portion of the inner wall 3003. The ramp 3005 is sloped from the front end to the rear end along a distance of the inner wall 3003. A front stop 3006 is positioned at or near the front end of the ramp 3005, and is configured for abutment with the torque transfer element 2701. The rear end of the ramp 3005 may be near to or aligned with the rear wall 3001 of the interior 3002. A rear stop 3007 is configured for abutment of the torque transfer element 2701 and is positioned at or near the rear end of the ramp 3005, or the rear stop 3007 may be separated from the rear end of the ramp 3005 by a radially flat section located between the rear end of the ramp 3005 and the rear stop 3007. Particular features of the ramp 3005, front stop 3006 and rear stop 3007 may include those features substantially as described for the First Example.

The rear wall 3001 of the actuator 2602 is configured as an inwardly projecting rim from the rear end of the actuator side wall, which defines the interior 3002 at a central position, such that the rear wall 3001 is an annular rim or flange about the central rear opening. Thus the rear wall 3001 serves to retain the torque transfer element 2701 within the interior of the actuator and the rear opening serves to allow the communication of the torque transfer element 2701 with parts of the assembly outside of the interior 3002 of the actuator 2602.

A covering cap 2604 is configured to sealingly engage with the front end of the actuator 2602. The covering cap 2604 is configured to connect a breathing gases tube 2605 to the actuator 2602 in particular and the adjustment assembly 2601 more generally. The covering cap 2604 is substantially annulus shaped, and has an outer perimeter configured to seal with the front end of the actuator 2602. In an example, the covering cap includes an opening configured to sealingly engage with the breathing gases tube 2605. Inwardly projecting connecting flanges 2706 may be positioned on the inner sides of the opening of the covering cap 2604, configured to assist in connecting with the breathing gases tube 2605 to the covering cap 2604.

The actuator 2602 is thus configured to include a gases flow path 2707, receiving the gases flow path 2707 from a front end of actuator 2602. The gases flow path 2707 may start from the breathing gases tube 2605 and pass through the interior 3002 of the actuator 2602. Gases flow path 2707 may be in direct contact with the interior 3002 or isolated from the interior 3002 by the breathing gases tube 2605.

### Torque Transfer Element

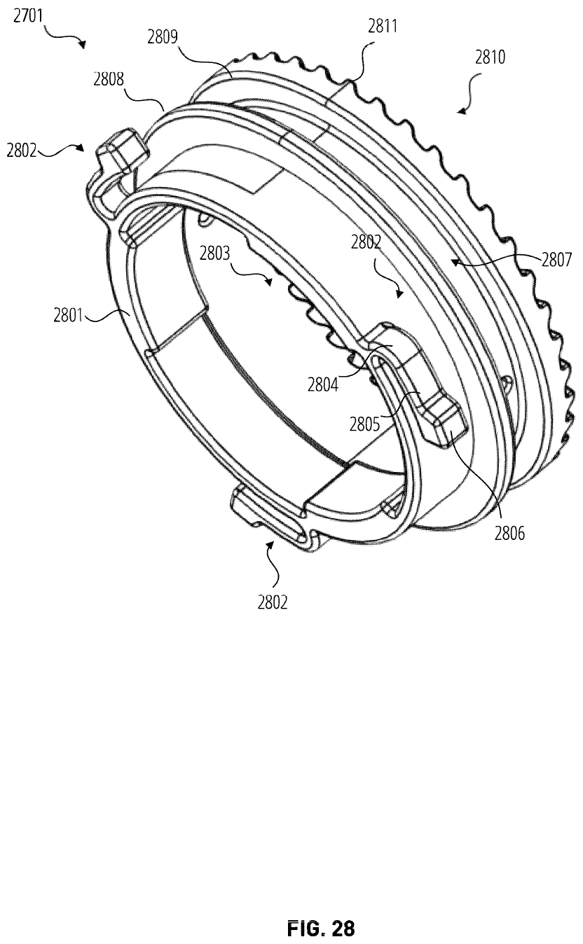

The torque transfer element 2701, shown in FIG. 28 and FIG. 29, includes a central body 2801 and one or more radially extending arms 2802 from the central body 2801. The central body 2801 may be a generally cylindrical body connected to the arms 2802. The central body 2801 includes a central aperture 2803 through which the gases flow path 2707 traverses, either in direct contact with the torque transfer element 2701 or isolated from the torque transfer element 2701. The construction of arm 2802 is substantially the same as described above for the First Example. Arm 2802 includes a proximal portion 2804 integrally formed with, or rigidly connected to, the central body 2801, and a distal portion 2805 integrally formed with, or rigidly connected to, the proximal portion 2804. A distal protrusion 2806 may be positioned substantially at an end portion of the distal portion 2805.

The torque transfer element 2701 is positioned in the interior 3002 of the actuator 2602, and the actuator 2602 and torque transfer element 2701 are substantially coaxial. The torque transfer element 2701 and interior 3002 are sized such that the distal portions 2805 of the arms 2802 and/or the distal protrusions 2806 abut with the contours 3004 of the inner wall 3003 of the actuator 2602. The torque transfer element 2701 and interior 3002 are further sized such that the torque transfer element 2701 is able to move along its axis of rotation within the interior 3002. This allows the axial movement of the torque transfer element as arms 2802 run along the ramp 3005.

Both axial and rotational movement of the torque transfer element 2701 is produced by the interaction between the distal protrusion of the arm and the contours of the inner wall of the interior of the actuator. The distal protrusions 2806 of the arm 2802 are configured to run along the surface of the ramp 3005, and to abut against the front stop 3006 and rear stop 3007. In this way, the rotation of the actuator 2602 is communicated to the torque transfer element 2701 via the contours of the actuator 2602 and in particular the front stop 3006 and rear stop 3007.

### Rotatable Member

As shown in FIG. 28 and FIG. 29, to the rear of the torque transfer element 2701 there is a coaxially positioned rotatable member 2702 for winding, storing, and unwinding the tension element 2608. The rotatable member 2702 may be integrally formed or connected with a rear end of the torque transfer element 2701, where the torque transfer element 2701 is positioned to the front of the rotatable member 2702. The connection may be a rigid connection, or the rotatable member 2702 may be integrally formed with the rear end of the torque transfer element 2701. The rotatable member 2702 and the torque transfer element 2701 may be keyed to ameliorate or prevent relative rotation between the components. The rotatable member 2702 includes an annular channel 2807 into which the tension element may be wound, stored and/or unwound. Rotation of the rotatable member 2702 in a first direction winds the tension element about the annular channel 2807 and rotation of the rotatable member 2702 in a second direction unwinds the tension element 2608 from the annular channel 2805. In this way, the effective length of the tension element 2608 that is outside of the housing can be shortened or extended by rotation of the rotatable member 2702. In some examples, the rotatable member 2702 may have two or more annular channels 2807. Each annular channel 2807 may be configured to receive one or more tension elements 2608. In some examples, two or more annular channels 2807 may have different diameters.

An outwardly protruding rim 2808 is positioned on a front side of the annular channel 2807, and is configured to rotate against or near the inner wall 3003 of the housing 2705. The torque transfer element 2701 may be notionally delineated from the rotatable member 2702 at the outwardly protruding rim 2808. FIG. 28 shows the torque transfer element 2701 and the rotatable member 2702 formed as an integral unit, sharing the same axis of rotation.

A central aperture 2803 located in the torque transfer element 2701 and rotatable member 2702 is configured to define a portion of the gases flow paths 2707, and may further accommodate the conduit 2704 or breathing gases tube 2605 extending therethrough. Both torque transfer element 2701 and rotatable member 2702 are configured to rotate about a gases flow path 2707. The central aperture 2803 has a substantially constant diameter within the interiors of both torque transfer element 2701 and rotatable member 2702. That is, the diameter of the central aperture 2803 in the rotatable member 2702 and torque transfer element 2701 is substantially constant. In part, this is to allow a conduit 2704 or breathing gases tubes 2605 of substantially consistent diameter to extend through the central aperture 2803.

The rear end of the rotatable member 2702 may include a flange 2809 positioned on a rear side of the annular channel 2807. A first locking element 2810 positioned on the rear side of the rotatable member 2702, such as on the rear side of the flange 2809. The purpose of the first locking element 2810 is to control the rotation of the rotatable member 2702 by interacting with a complementary second locking element 3301 mounted on the base 2703. For example, the first locking element 2810 includes a plurality of rearwardly projecting teeth 2811 arranged on a rear side of the flange 2809. The rearwardly projecting teeth 2811 are arranged around the perimeter of the flange. They may be equally spaced and sized, and may be asymmetrically shaped, such as uniformly slanted off-center. The angle of the off-center slant imparts a directional bias to the first locking element 2810 and the interaction of the locking element with the base 2703, such that the teeth may be rotated against the second locking element 3301 with less resistance in one direction compared to the opposite direction. Varying the angle of the off-center slant affects the resistance to rotation. Each of the rearwardly projecting teeth 2811 are angled at about 30 degrees and have a depth of about 0.4 mm. The first locking element 2810 and its interaction with the second locking element 3301 is substantially the same as the first and second locking elements described above for the First Example.

### Housing

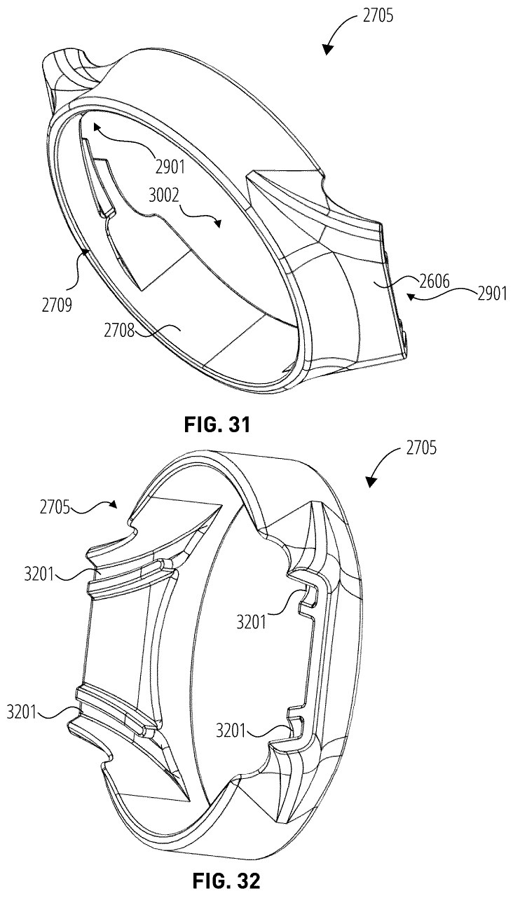

As shown in FIG. 31 and FIG. 32, the adjustment assembly 2601 includes a housing 2705 for retaining the rotatable member 2702 and stored tension elements 2608.

The housing 2705 includes an interior side wall 2708 that at least partially defines an interior 3002 for retaining the rotatable member 2702. A front end 2709 of the housing includes a front opening facing the actuator 2602 and a rear end 2710 of the housing includes a rear opening facing the base 2703.

The front end 2709 of the housing 2705 is configured to form a rotatable connection with a rear end of the actuator 2602, such that the actuator 2602 may rotate relative to the housing which is held in a fixed position. The headgear adjustment assembly 2601 may include an intervening seal ring (e.g., an O-ring) between a peripheral edge of the rear end of the actuator and the front end of the housing 2705.

Left and right side arm covers 2606 are positioned on opposing sides of the housing main body. Side arm covers 2606 cooperate with side arms 3302 on the base 2703 to define left and right side channels 2901 for receiving the tension elements 2608 and to direct the tension elements 2608 from the interior of the housing towards straps of the headgear. The side channels 2901 have a tapering cross section as the tension element pathway narrows as it extends away from the interior of the housing 2705. The left and right side arm covers 2606 include top and bottom grooves 3201 for receiving a complementary shaped rib on the base. The grooves 3201 on the side arms and the ribs on the base interconnect to assist in connecting and correctly positioning the housing 2705 and the base 2703.

The rear end 2710 of the housing 2705 connects to the base 2703, such that the rear opening of the housing communicates with an opening on the base 2703.

### Base

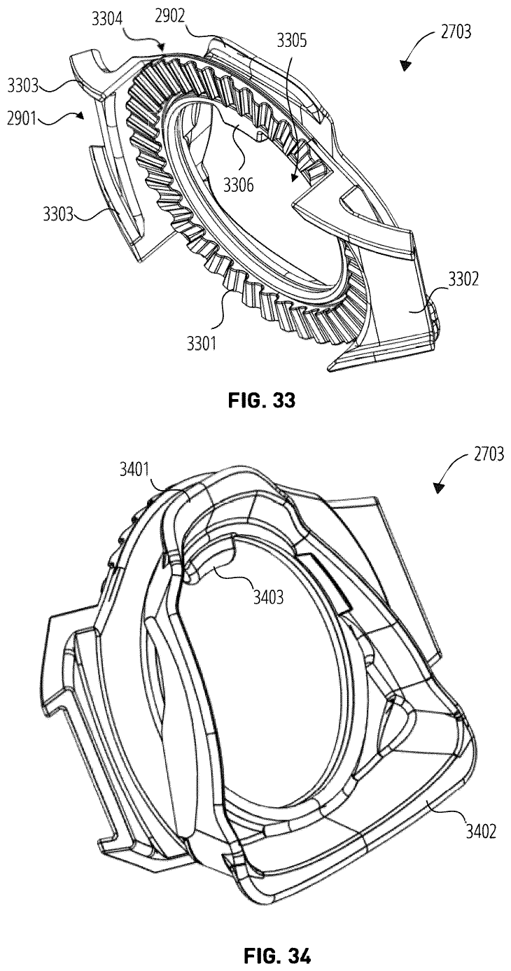

The base 2703, shown in FIG. 33 and FIG. 34, is positioned to the rear of the rotatable member 2702 and housing 2705, and includes a frame for supporting a face seal 2607, left and right side arms 3302, and a second locking element 3301.

The left and right side arms 3302 cooperate with the left and right side arm covers 2606 on the housing. As described above, the side arms 3302 of the housing 2705 and the side arm covers 2606 of the base 2703 interconnect by the engagement of top and bottom ribs 3303 on the base 2703 with the respective top and bottom grooves 3201 on the housing 2705. The housing 2705 and base 2703 thus cooperate to define a guide channel for directing the tension element 2608 to the headgear. The guide channels retain the tension elements along a path from the tension element apertures in the housing towards the side arms of a headgear.

The base 2703 includes a main body 3304 including a central opening 3305, and the second locking element 3301. The central opening 3305 is surrounded by the second locking element 3301. The second locking element 3301 is shaped and sized to complementarily match the shape and size of the first locking element 2810. In this regard, the second locking element 3301 includes a plurality of forwardly projecting teeth arranged on a front side of the main body 3304. Each of the plurality of teeth are circularly arranged around the perimeter of the central opening 3305, are equally spaced and sized, and are uniformly slanted off-center. The angle of the off-center slant is reciprocal/inverse to the angle of the off-center slant of the teeth in the first locking element.

The second locking element 3301 and its interaction with the first locking element 2810 is substantially the same as that described above for the First Example, except as shown or described.

The base 2703 includes one or more parts for connecting to a seal 2607 (shown in FIG. 26 as a nasal pillows type seal). At the rear end of the base 2703, a top flange 2902 and a bottom flange 3402 are configured to engage with the seal 2607, such that a front opening of the seal 2607 may envelope around, and sealingly engage with, the flanges and outer sides of the base.

### Gases Flow Path

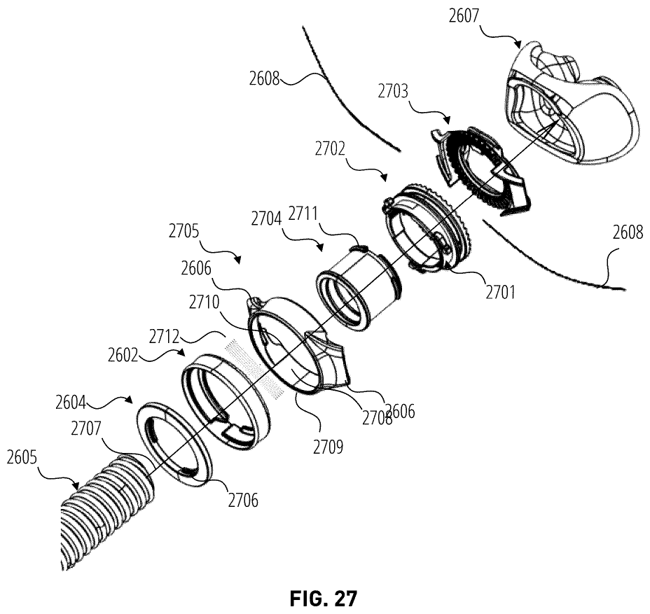

A gases flow path 2707 may pass through the adjustment assembly 2601 in a substantially straight line (see FIG. 27). For example, the gases flow path 2707 may extend from a front end to a rear end of the adjustment assembly 2601 in a substantially straight line. The gases flow path 2707 may at least partially extend along a central axis of rotation of the actuator 2602, torque transfer element 2701, rotatable member 2702 and base 2703. The gases flow path 2707 is unimpeded by any parts, especially moving parts, of the assembly 2601.

### Breathing Gases Tube and Conduit

Breathing gases tube 2605 extends through the opening of the covering cap 2604 and at least into a portion of the interior of the actuator 2602. In another example, the breathing gases tube 2605 may extend through the interior of the actuator 2602 and the housing 2705. In another example, the breathing gases tube extends through openings in the actuator, housing, torque transfer element and optionally the base 2703. In another example, the breathing gases tube 2605 may be coupled with an elbow coupled with the covering cap 2604, actuator 2602, and/or conduit 2704.

The breathing gases tube 2605 sealingly engages with the conduit 2704. The breathing gases tube 2605 and conduit 2704 may be joined by overmolding. Alternatively, the breathing gases tube 2605 may include a ridge (e.g., a helical ridge) on an outer side thereof, and conduit 2704 may include a helical thread on an inner wall, where the ridge and thread are configured to engage with each other in a screw type relationship.

Conduit 2704 is configured to engage at a rear end thereof with the base 206. Conduit 2704 includes a flange 2711 at a rear end thereof for engaging with the base 2703 around the central opening 3305. Flange 2711 may be configured to interlock with lip 3306, such that flange 2711 may be connected to base 2703 by rotating the flange 2711 behind lip 3306. Accordingly, conduit 2704 can sealingly engage with the breathing gases tube 2605 at a front end and the central opening 3305 of the base at a rear end, thus bringing the gases flow path 2707 into fluid communication with the seal 2607, and isolating the gases flow path 2707 from the parts of the adjustment assembly 2601, and in particular from the moving parts of the assembly such as the tension element 2608, torque transfer element 2701, rotatable member 2702 and actuator 2602.

### Biasing Member

As with the First Example, axial movement of the torque transfer element 2701 in the actuator interior is in part controlled by the axial biasing of the torque transfer element 2701 rearwardly (towards the rear wall of the actuator 2602), that is, axial biasing of the torque transfer element 2701 towards the rear end of the ramp 3005. Axial biasing can be produced with springs or the use of other resilient members that exert an axial urging force on the torque transfer element 2701.

Accordingly, biasing member 2712 is positioned in the interior of the actuator 2602 between the covering cap 2604 and the torque transfer element 2701 to exert a rearward biasing force on the torque transfer element 2701. However, biasing member 2712 may be positioned in other parts of the assembly 2601 and still provide the biasing force. For example, the biasing member 2712 may be located in the housing 2705 to the rear of the rotatable member and/or torque transfer element 2701. The biasing member 2712 performs substantially the same function as described in the First Example.

### Tension Element

The tension element 2608 is for transferring the tensioning forces from the rotatable member 2702 onto the headgear. One or more tension elements 2608 are configured to engage with the rotatable member 2702 so that they may be wound, stored, and unwound from the rotatable member 2702, and the annular channel 2807 in particular. The tension element 2608 and/or headgear may be substantially as described for the First Example.

### THIRD EXAMPLE

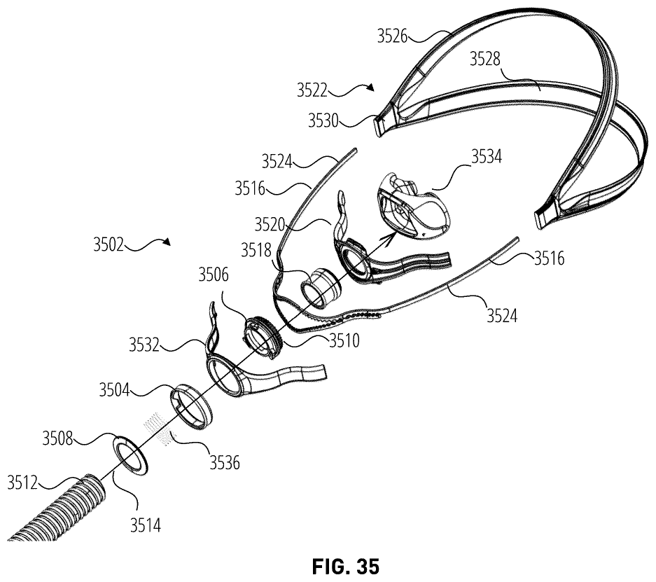

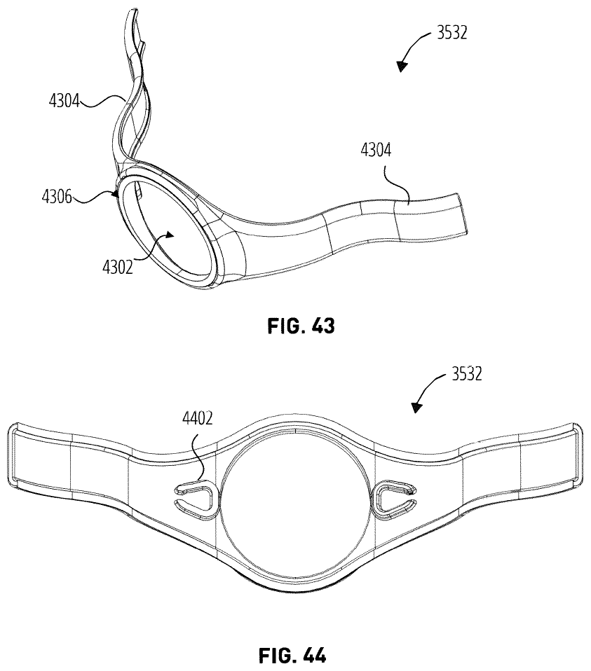

With reference to FIG. 35 to FIG. 45, there is provided a Third Example of an adjustment assembly 3502. The adjustment assembly 3502 may include an actuator 3504, torque transfer element 3506, rotatable member 3510, and base 3520. Adjustment assembly 3502 includes a rack and pinion assembly.

### Actuator

As best shown in FIG. 37, an actuator 3504 is positioned at a front part of the assembly, and includes an outer surface 3702 and a central recess defining an interior including an interior wall 3704 and a rear wall 3706. As described in previous examples, the outer surface 3702 is configured to be actuated by a user, and in particular is configured to be rotated clockwise and counter-clockwise by a user's fingers.

The outer surface 3702 includes a generally annular profile, and includes gripping features including a concave impression along a middle portion of the outer surface, such that the diameter of the outer surface is lower towards the middle and greater towards front and rear portions of the outer surface.

The actuator 3504 includes a front end 3708, a rear end 3710, and an interior defined in part by an interior wall 3704 and the internal space between the front end 3708 and rear end 3710. The interior is configured to at least partially house the torque transfer element 3506.

The interior wall 3704 includes at least one inwardly protruding contour 3712 configured to interact with the torque transfer element 3506. As with other examples described herein, a function of the contour 3712 is to translate rotational movement of the actuator 3504 to axial movement in the torque transfer element 3506 in the interior. For this, the contour 3712 includes a ramp 3714 along a portion of the interior wall 3704. The ramp 3714 is sloped from the front end 3708 to the rear end 3710 along a distance of the interior wall 3704.

The rear end of the ramp 3714 may be near to or aligned with the rear wall 3706 in the interior. A rear stop 3716 is positioned at or near the rear end of the ramp 3714, or the rear stop 3716 may be separated from the rear end of the ramp 3714 by a radially flat section located between the rear end of the ramp 3714 and the rear stop 3716, as shown in the drawings. The face of the rear stop 3716 is angled or sloped slightly away from the normal (relative to a radial line from the central axis). Alternatively, the rear stop 3716 may be configured as a flat face at right angles to the interior wall 3704.

In contrast to the First and Second Examples, the actuator 3504 as shown in FIG. 35 to FIG. 45 does not include a front stop for abutting the distal protrusion 3602 at the top end of the ramp 3714. However, a front stop could be provided if desired. In this example, an upper limit to axial movement of the torque transfer element 3506 is defined by either the top end of the ramp 3714 and/or by a top wall of a covering cap 3508.

When a user rotates the actuator 3504 in a tightening direction, the rear stops 3716 abut against the distal portions of the torque transfer element 3506, which brings the actuator 3504 into rotational communication with the torque transfer element 3506 such that torque is transferred from the actuator 3504 to the torque transfer element 3506. Torque is transferred from the torque transfer element 3506 to the rotatable member 3510, which transfers the torque to the tension element 3516 connected thereto which in turn applies tightening force to the headgear 3522. As the headgear 3522 is tightened, the amount of torque required to further tighten the headgear 3522 increases. The torque transfer element 3506 is configured to slip over the contours 3712 of the actuator 3504 when the tightening force applied by the user reaches a threshold, like the mechanism described in the First Example. In this way, the assembly may be configured to prevent overtightening of the headgear by limiting the amount of torque a user can apply to the tension elements 3516 via the actuator 3504.

The ramp 3714 and rear stop 3716 may be provided as a unitary projection on the interior wall 3704. Alternatively, the ramp 3714 and rear stop 3716 may be provided as separate projections on the interior wall 3704.

The ramp 3714 and rear stop 3716 may be integrally formed with the body of the actuator 3504. Alternatively, any of the ramp 3714 and rear stop 3716 may be separate to, and rigidly connected to, the interior wall 3704.

The rear wall of the actuator 3504 is configured as an inwardly projecting rim from the rear end of the side wall of the actuator 3504. The rear wall includes a rear opening at a central position, such that the rear wall is an annular rim or flange about the central rear opening. Thus the rear wall serves to retain the torque transfer element 3506 within the interior of the actuator 3504 and the rear opening serves to allow the communication of the torque transfer element 3506 with parts of the assembly outside of the interior of the actuator 3504.

A covering cap 3508 is configured to sealingly engage with the front end of the actuator 3504. The covering cap 3508 is configured to connect the breathing gases tube 3512 to the actuator 3504 in particular and the adjustment assembly 3502 more generally.

The covering cap 3508 is substantially annulus shaped, and has an outer perimeter configured to seal with the front end of the actuator 3504. In an example, the covering cap includes an opening configured to sealingly engage with a breathing gases tube 3512. Inwardly projecting connecting flanges may be positioned on the inner sides of the opening of the covering cap 3508, configured to assist in connecting with the breathing gases tube 3512.

The actuator 3504 is configured to receive a gases flow path 3514 from a front end thereof. A gases flow path 3514 begins in the breathing gases tube and passes through the interior of the actuator 3504.

### Torque Transfer Element and Rotatable Member

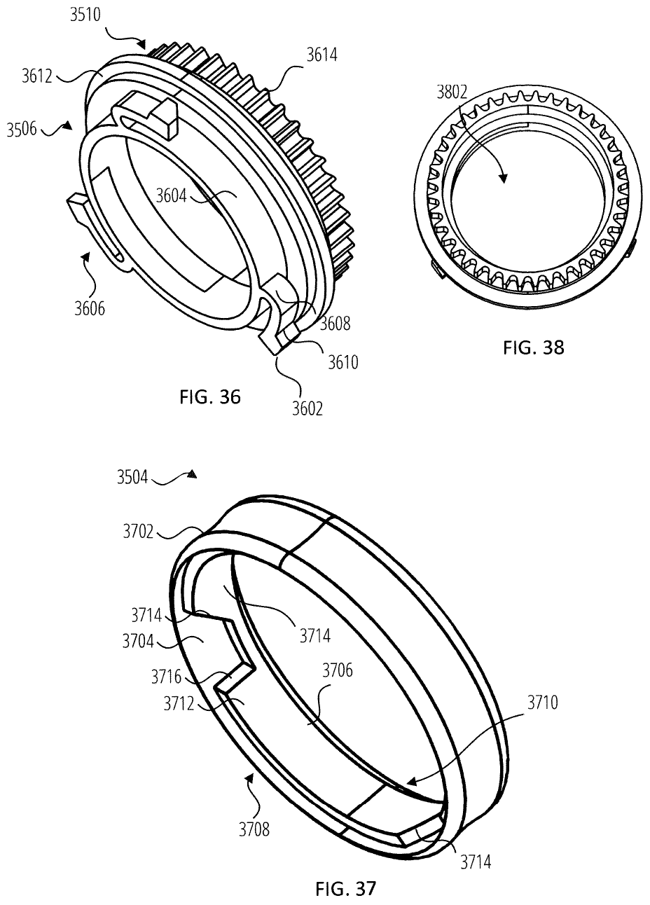

As with the previous example, and as shown in FIG. 36, the torque transfer element 3506 includes a central body 3604 and one or more radially extending arms 3606 from the central body 3604. The central body 3604 includes a generally cylindrical body that attaches to the arms. The central body 3604 may include a central aperture 3802. Each arm 3606 includes a proximal portion 3608 integrally formed with, or connected to, the central body 3604, and a distal portion 3610 integrally formed with, or connected to, the proximal portion 3608.

The torque transfer element 3506 is positioned in the interior of the actuator 3504, and the actuator 3504 and torque transfer element 3506 share the same axis of rotation. The torque transfer element 3506 and interior are sized such that the distal portions 3610 of the arms 3606 and/or the distal projections of the arms 3606 abut with the contours 3712 of the interior wall 3704 of the actuator 3504. The torque transfer element 3506 and interior are further sized such that the torque transfer element 3506 is able to move along its axis of rotation within the interior. This allows the axial movement of the torque transfer element 3506 as it runs along the ramp 3714.

Both axial and rotational movement of the torque transfer element is affected by the interaction between the distal protrusion of the arm and the contours of the inner wall of the interior of the actuator. The distal portions and/or protrusions of the arm are configured to run along the surface of the ramp, and to abut against the front and rear stops. In this way, the rotation of the actuator is communicated to the torque transfer element.

As shown in FIG. 36 and FIG. 38, the rotatable member 3510 is connected to the rear end of the torque transfer element 3506. The rotatable member 3510 is configured to drive one or more tension elements 3516 in a first direction or a second direction as the rotatable member 3510 is rotated in a first direction or a second direction, respectively. In this way, the effective length of the tension element 3516 can be shortened or extended by rotation of the rotatable member 3510. In this example, the tension elements 3516 and the rotatable member 3510 operate in a rack and pinion arrangement.

The torque transfer element 3506 may be notionally delineated from the rotatable member 3510 at an outwardly protruding rim 3612 positioned on a front side of the teeth 3614. The rim 3612 is configured assist in retaining the tension element 3516 in alignment with the teeth 3614 and rotatable member 3510.

The torque transfer element 3506 and the rotatable member 3510 are formed as an integral unit, sharing the same axis of rotation, and configured to rotate about a gases flow path 3514. The torque transfer element 3506 is connected to the rotatable member 3510, where the torque transfer element 3506 is positioned to the front of the rotatable member 3510. The connection may be a rigid connection, or the torque transfer element 3506 may be integrally formed with the rotatable member 3510. A central aperture 3802 in the torque transfer element 3506 and rotatable member 3510 is configured to include a portion of the gases flow path 3514, and may further include a conduit 3518 or breathing gases tube 3512 extending therethrough. The central aperture 3802 has a substantially consistent diameter within the interiors of both parts. That is, the diameter of the central aperture 3802 in the rotatable member 3510 and torque transfer element 3506 is substantially coincident. In part, this is to allow the conduit 3518 or breathing gases tubes breathing gases tube 3512 (of substantially consistent diameter) to extend through the central aperture 3802.

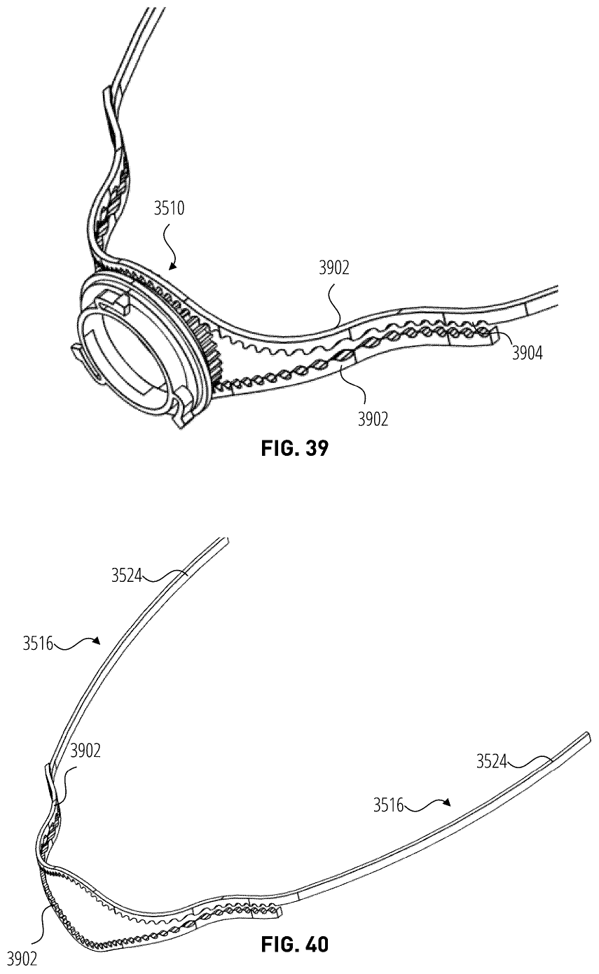

As shown in FIG. 39, the rotatable member 3510 is mounted between the one or more tension elements 3516. The rotatable member 3510 and the tension element 3516 can be positioned within or on the base 3520. The ends of the one or more tension element 3516 can connect to or be integrated into the headgear 3522. The one or more tension elements 3516 can be flexible enough to wrap slightly around the rotatable member 3510 to provide more purchase between the tension element 3516 and the rotatable member 3510 and bring the tension element 3516 into alignment for generally symmetrical headgear attachment.

In this example, the rotatable member 3510 and tension element interact as a pinion and rack. The rotatable member 3510 includes an annular body including a plurality of outwardly projecting spaced apart teeth 3614. As the rotatable member 3510 rotates, the teeth 3614 move the rack 3902. In this manner, the rack 3902 can be used to adjust the tension on the headgear. In some examples, the rotatable member 3510 may have two or more annular bodies each including a plurality of outwardly projecting spaced apart teeth 3614. Each annular body may be configured to receive one or more tension elements. In some examples, two or more annular bodies may have a different diameter.

Any suitable locking mechanism can be used to lock the position of the headgear 3522, the rack 3902, the rotatable member 3510 and/or the actuator 3504. For instance, a pin or the like can be used to inhibit rotation of the rotatable member 3510 and/or the actuator 3504. In some configurations, a friction break, a clamping mechanism, a cammed break member or the like can be used to inhibit movement of one or more of the headgear 3522, the rack 3902, the rotatable member 3510 and/or the actuator 3504.

### Base and Face Plate

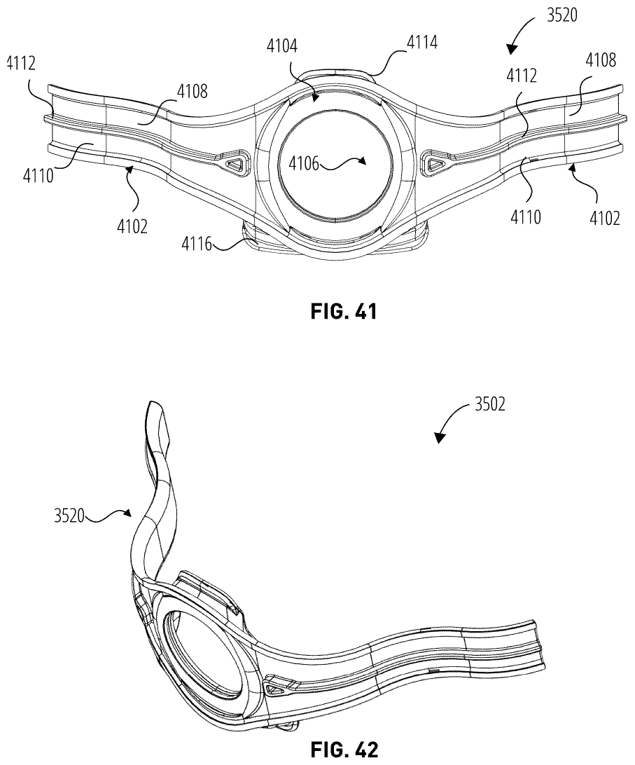

The base 3520, shown for example in FIG. 41, is positioned to the rear of the rotatable member 3510 and includes left and right side arms 4102 extending laterally outwardly from a main body 4104 including a central opening 4106. The base 3520 may be formed of a rigid material such as a thermoplastic, polycarbonate, or the like.

A rear side of the face plate 3532, shown in FIG. 44, connects to the base 3520, such that the central opening 4302 of the face plate communicates with the central opening 4106 on the base 3520. The face plate 3532 is configured to interact with the base 3520 to form an internal housing that retains one or more tension elements 3516, and the rotatable member 3510. Face plate 3510 thus comprises a central opening 4302, and left and right side arms 4304 including a shape and curvature corresponding with the shape and curvature of the left and right side arms 3526 of the base 3520.

A front end of the face plate 3532 is configured to form a rotatable seal with a rear end of the actuator 3702, such that the actuator 3504 may rotate relative to the face plate 3532. To better achieve a rotatable seal, the adjustment assembly 3502 may include an intervening seal ring (e.g., an O-ring) between a peripheral edge of the rear end of the actuator and the front end of the face plate 3532.

The left and right side arms 4102 on the base 3520 cooperate with the left and right side arms 4304 on the face plate 3532. The side arms of the face plate 3532 and the base 3520 contact each other at top and bottom portions thereof to define a left and right guide channels for directing the tension element along a path from the assembly to the left and right sides of the headgear 3522. The guide channels retain the tension elements along a path from the tension element apertures in the housing towards the side arms of a headgear. Left and right guide channels are further defined into a top guide channel 4108 and a bottom guide channel 4110 by one or more ridges 4112 disposed along a median portion of the front side of the side arms on the base 3520. The one or more ridges 4112 act to keep the first and second tension torque transfer elements 3506 separate from one another, for example as they move in opposite directions from each other as they are driven in first and second directions. One or more ridges 4402 may be positioned on side arms 4304 of the face plate to cooperate with the ridges 4112 on the base 3520. For example, FIG. 41 shows left and right side ridges 4112 on the base 3520 including elongate portion and a widened portion located at an inner end thereof, which are overlapped by the ridges 4402 (see FIG. 44) on the face plate 3532. The ridges thus have a function of interlocking the face plate and the base in addition to defining the guide channels for the tension elements 3516. The guide channels have a tapering cross section as the tension element pathway narrows as it extends outwardly from the assembly.

Side arms may be configured such that the guide channels extend outwardly along the left and right sides of a user's face along at least a portion of the user's cheek. This is to prevent or ameliorate forces transferring from the tension elements 3516 to the sides of a user's face as the tension elements are tightened.

The base 3520 includes one or more parts for connecting to a seal 3534. At the rear end of the base 3520, a top flange 4114 and a bottom flange 4116 are configured to engage with the seal 3534, such that a front opening of the seal 3534 may envelop, and sealingly engage with, the flanges and outer sides of the base 3520.

### Biasing Member

As with the First and Second Example, axial movement of the torque transfer element 3506 in the actuator interior is in part controlled by the axial biasing of the torque transfer element 3506 rearwardly (towards the rear wall of the actuator 3504), that is, axial biasing of the torque transfer element 3506 towards the rear end of the ramp 3714. Axial biasing can be produced with springs or the use of other resilient members that exert an axial urging force on the torque transfer element 3506.

Accordingly, biasing member 3536 is positioned in the interior of the actuator 3504 between the covering cap 3508 and the torque transfer element 3506 to exert a rearward biasing force on the torque transfer element 3506. However, biasing member 3536 may be positioned in other parts of the adjustment assembly 3502 and still provide the biasing force. For example, the biasing member 3536 may be located to the rear of the rotatable member 3510 and/or torque transfer element 3506.

### Tension Elements

The tension element 3516 is for transferring the tensioning forces from the rotatable member 3510 onto the headgear 3522. One or more tension elements 3516 are configured to engage with the rotatable member 3510. In this example, first and second tension elements 3516 each include rack 3902 located along an end portion of the tension element including a series of teeth 3904. At least a portion of the rack 3902 is retained in the guide channels. The tension elements may be formed of a rigid or semi rigid material. The rigidity of the tension elements allows the headgear to be loosed by driving the tension elements with the pinion to lengthen the effective length of each tension element. The rack 3902 may be sufficiently flexible to be driven about the rotatable member 3510. Part of the tension element 3516 may be integrally formed with side straps 3524.

The first and second tension elements are configured to be mounted on the rotatable member 3510 at upper and lower positions, such that the first tension element is driven across an upper side of the rotatable member 3510 in a first direction and the second tension element is driven across a lower side of the rotatable member 3510 in the opposite direction. Accordingly, first and second tension elements may be mirror images of each other. Rotation of the rotatable member 3510 in thus drives the first and second tension elements in opposing directions, so that they are both retracted (or extended) simultaneously. When the tension elements 3516 are connected to the headgear, rotation of the rotatable member 3510 thus effects a respective tightening or loosening of the headgear.

### Engaged/Disengaged Configuration

As with other Examples, torque transfer element 3506 is translatable along its axis of rotation between an engaged configuration and a disengaged configuration. In this Example, however, the engaged configuration includes the engagement of the rotatable member 3510 with the tension element 3516 and the disengaged configuration includes the disengagement of the rotatable member 3510 from the tension element 3516.

The engaged configuration and disengaged configuration are thus defined by the relative position of the rotatable member 3510 and the tension element 3516. The engaged and disengaged configurations may be further defined by the relative position of the arms 3606 of the torque transfer element 3506 on the ramp 3714, where the tension element 3516 is positioned at a rear or bottom end of the ramp 3714 in the engaged configuration.

In the engaged configuration, the rotatable member 3510 may be rotated in a tightening direction so as to drive the tension element 3516 in the tightening direction. Restoring forces exerted by the tensioning elements on the rotatable member 203 may be resisted by a locking mechanism (not shown) that can be used to lock the position of the headgear 3522, the rack 3902, the rotatable member 3510 and/or the actuator 3504.

In the disengaged configuration, the rotatable member 3510 is axially displaced from engagement with the rack 3902. The tension element 3516 is therefore disengaged from the rotatable member 3510 and not constrained by any locking forces. The tension element 3516 may freely slide in the tightening or untightening direction. The user may loosen or untighten the tension elements by rotating the actuator 208 in the untightening direction, or alternatively the user may pull directly on the tension elements or headgear straps attached thereto so as to directly loosen the tension elements.

### Headgear

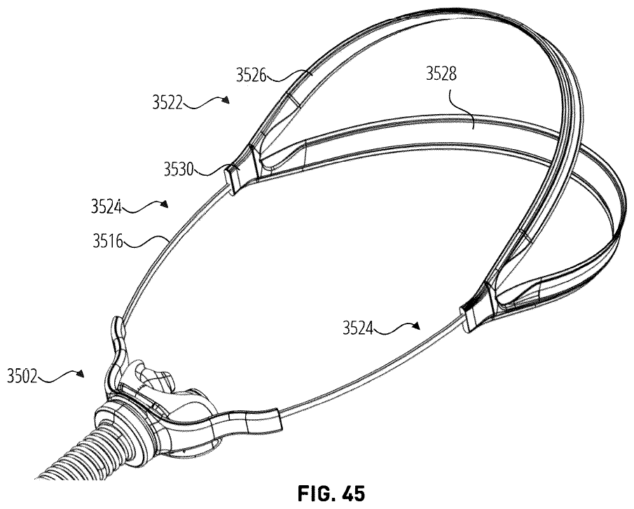

As shown in FIG. 45, the headgear 3522 includes one or more side straps 3524, a top strap 3526 and a back strap 3528 and one or more pathways for receiving the tension element. Each of the one or more side straps 3524 connect at a distal end thereof to one or more junction portions 3530. The top strap 3526 and back strap 3528 each connect to left and right side junction portions 3530 at ends thereof.

The tension element 3516 may connect to a headgear strap, or portion. Alternatively, or additionally, the side straps 3524 may be integrally formed with or connected to the tension elements 3516. For example, left and right side straps 3524 may extend from the respective junction portions 3530 towards the front of the face and join the corresponding left or right tension element 3516. In the example shown in FIG. 40, a tension element 3516 includes the side straps 3524 and the rack portion 3902 as an integral element. A portion of the side strap is shaped to extend along a line in part defined by the side of a user's face. An end of the side strap 3524 distal with respect to the top and rear straps may be formed to curve inwardly towards the adjustment assembly 3502 such that a portion of its distal end is configured to be retained within the guide channels and interior of the assembly. In one example, the tension element 3516 may be made of a rigid or semi-rigid material to maintain its curved shape when under tension, for example such that the tightening forces exerted on the tension element 3516 by the assembly are transferred to the headgear without overly squeezing or pinching the face of the user.

### Breathing Gases Tube and Conduit

In one example, the breathing gases tube 3512 extends through the opening of the covering cap 3508 and into at least a portion of the interior of the actuator 3504. In another example, the breathing gases tube extends through the interior of the actuator 3504 and the central aperture 3802 of the torque transfer element 3506 and optionally the base 3520.

In another example, the breathing gases tube 3512 sealingly engages with a conduit 3518. The breathing gases tube 3512 and conduit 3518 may have substantially similar features to those described for the Second Example.

As noted above, in any of these examples, the breathing gases tube 3512 may be configured to connect to the opening directly or via an elbow. There may be a swivel joint or a ball joint between the breathing gases tube 3512 and/or the opening and the elbow.

### Gases Flow Path

As with the Second Example, a gases flow path 3514 may pass through the adjustment assembly 3502 in a substantially straight line. The gases flow path 3514 may have substantially similar features to those described for the Second Example.

### FOURTH EXAMPLE

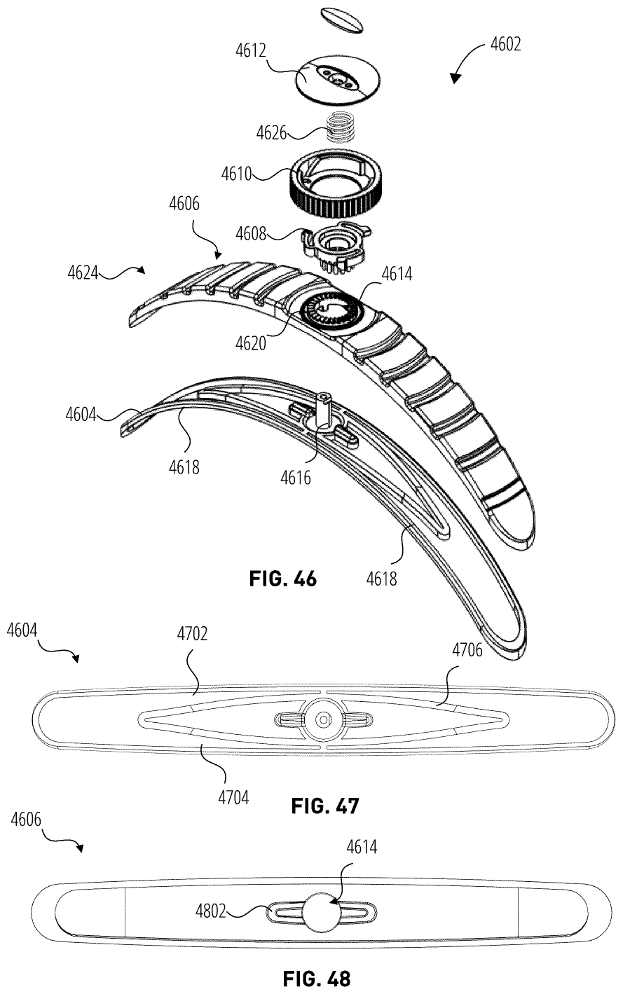

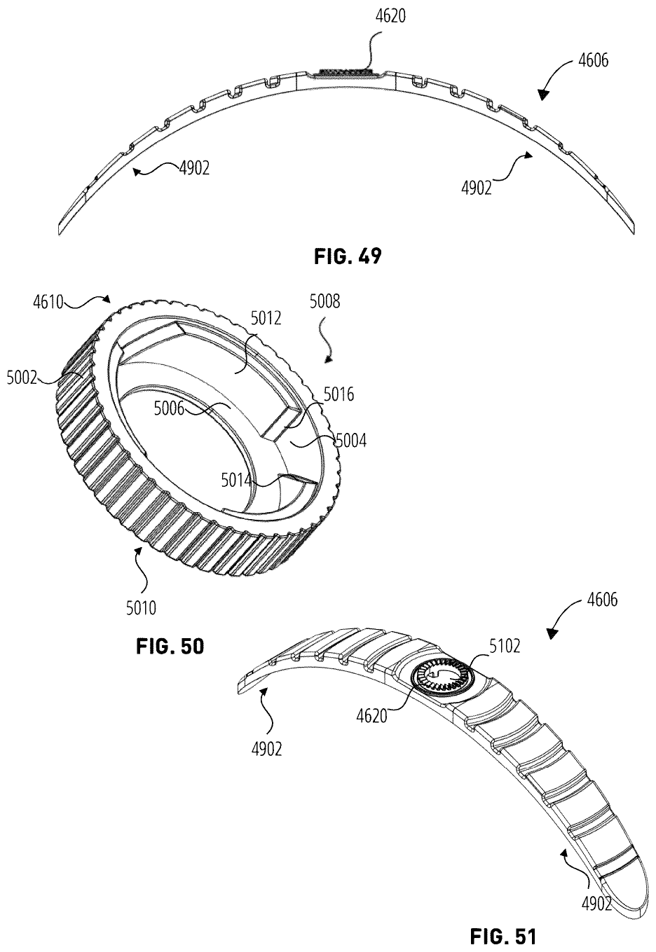

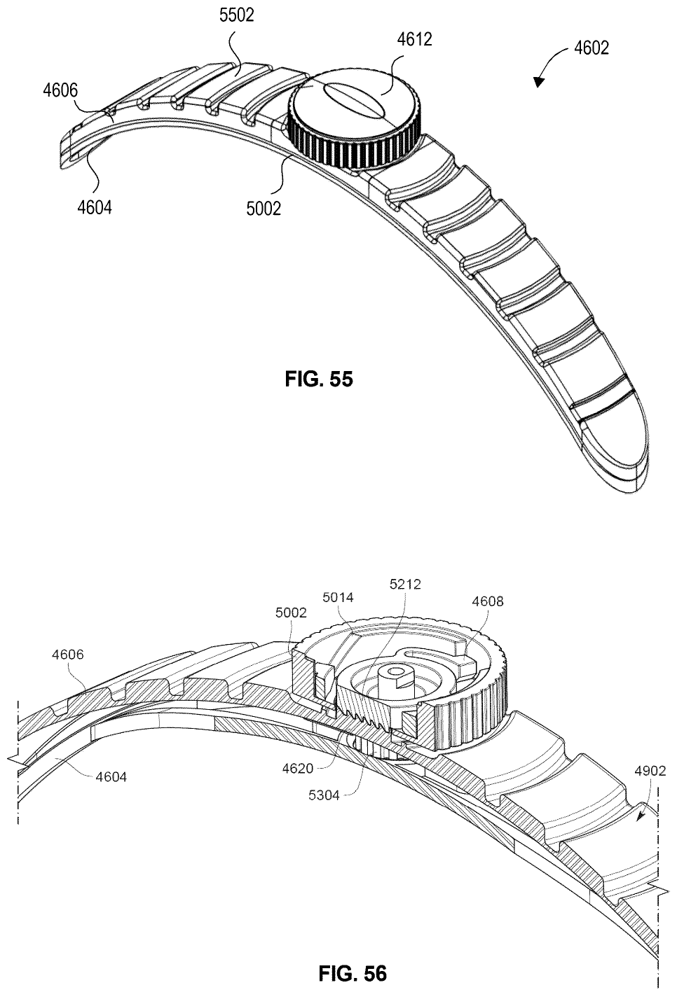

With reference to FIG. 46 to FIG. 57, there is provided a Fourth Example of an adjustment assembly 4602. The adjustment assembly 4602 may include an actuator 4610, torque transfer element 4608, rotatable member 5302, and base 4624. Adjustment assembly 4602 is described here with reference to a rack and pinion assembly.

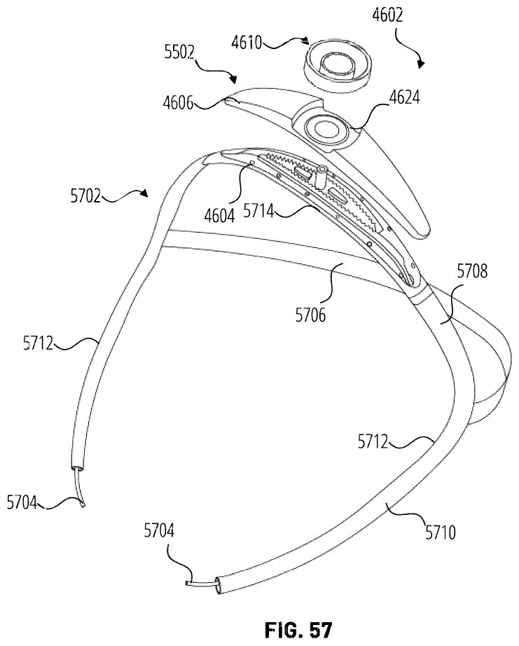

FIG. 46 shows the adjustment assembly 4602 configured to be positioned on a headgear strap. In particular, the assembly is configured to be positioned on a top headstrap or a rear headstrap of the headgear 5702 (FIG. 57 shows the adjustment assembly 4602 positioned on the top headstrap of headgear 5702). The assembly 4602 includes a housing 5502 including a lower body 4604 and corresponding upper body 4606, a torque transfer element 4608, an actuator 4610 and covering cap 4612. The adjustment assembly 4602 is configured to include one or more tension elements for transferring tension to the headgear so as to tighten or loosen the headgear. The assembly 4602 may be connected to a strap, for example by welding, adhesives or overmolding.

### Actuator

As best shown in FIG. 50, the actuator 4610 includes an outer surface 5002 constructed of a molded plastic material, and a central recess defining an interior including an interior wall 5004 and a bottom wall 5006. As described above, the outer surface 5002 is configured to be actuated by a user, and in particular is configured to be rotated clockwise and counter-clockwise by a user's fingers.

The outer surface 5002 includes a generally annular profile, and includes gripping features positioned thereon to improve gripping by the fingertips. FIG. 50 shows a plurality of ribs positioned longitudinally around the circumference of the outer surface 5002. In other examples, the outer surface 5002 may be knurled or scalloped.

The actuator 4610 includes a top end 5008, a bottom end 5010, and an interior defined in part by the interior wall 5004 and the internal space between the top end 5008 and bottom end 5010. The interior is configured to at least partially house the torque transfer element 4608.

The interior wall 5004 includes at least one inwardly protruding contour 5012 configured to interact with the torque transfer element 4608. As with other examples described herein, a function of the contour 5012 is to translate rotational movement of the actuator 4610 to axial movement in the torque transfer element 4608 in the interior. For this, the contour 5012 includes a ramp 5014 along a portion of the interior wall 5004. The ramp 5014 is sloped from the bottom end 5010 to the top end 5008 along a distance of the interior wall 5004.

The bottom end of the ramp 5014 may be near to or aligned with the bottom wall 5006 in the interior. A rear stop 5016 is positioned at or near the rear end of the ramp 5014, or the rear stop 5016 may be separated from the rear end of the ramp 5014 by a radially flat section located between the rear end of the ramp 5014 and the rear stop 5016. The face of the rear stop 5016 is angled or sloped slightly away from the normal (relative to a radial line from the central axis). Alternatively, the rear stop 5016 may be configured as a flat face at right angles to the interior wall 5004.

In contrast to other Examples described herein, the actuator 4610, as shown, does not include a separate front stop at the top end of the ramp 5014 (corresponding to front stops 3006, for example). In this example, an upper limit to axial movement of the torque transfer element 4608 is defined by either the top end of the ramp 5014 and/or the inner surface of a covering cap 4612.

When a user rotates the actuator 4610 in a tightening direction, the rear stops 5016 abut against the distal portions 5202 of the torque transfer element 4608, which brings the actuator 4610 into rotational communication with the torque transfer element 4608 such that torque is transferred from the actuator 4610 to the torque transfer element 4608. Torque is transferred from the torque transfer element 4608 to the rotatable member 5302, which transfers the torque to the tension element connected thereto which in turn applies tightening force to the headgear. As the headgear is tightened, the amount of torque required to further tighten the headgear increases. The torque transfer element 4608 is configured to deform and slip over the contours of the actuator when the tightening force applied by the user reaches a threshold as described in the First Example. In this way, the assembly may be configured to prevent overtightening of the headgear by limiting the amount of torque a user can apply to the tension elements via the actuator 4610.

The bottom wall 5006 of the actuator 4610 is configured as an inwardly projecting rim from the rear end of the interior wall 5004 of the actuator 4610. The bottom wall 5006 includes a rear opening at a central position, such that the bottom wall 5006 is an annular rim or flange about the central rear opening. Thus, the bottom wall 5006 serves to retain the torque transfer element within the interior of the actuator and the rear opening serves to allow the communication of the torque transfer element with parts of the assembly outside of the interior of the actuator.

A covering cap 4612 is configured to engage with the front end of the actuator 4610. The covering cap 4612 is substantially annulus shaped, and has an outer perimeter configured to seal with the top end of the actuator 4610.

### Torque Transfer Element and Rotatable member

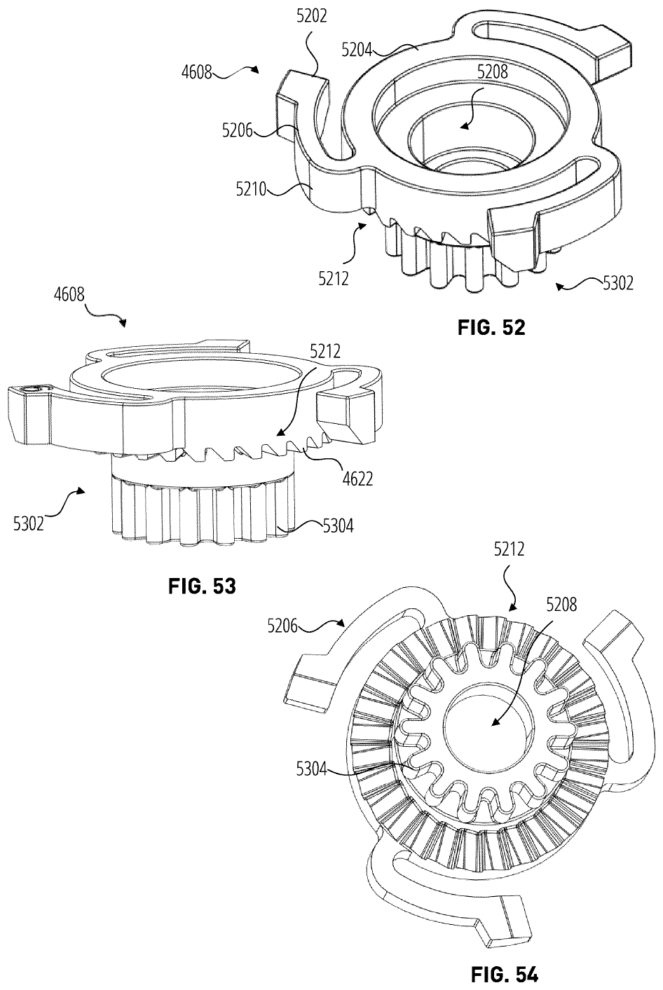

As best shown in FIG. 52, FIG. 53, FIG. 54, and FIG. 56, the torque transfer element 4608 includes a central body 5204 and one or more radially extending arms 5206 from the central body 5204. The central body 5204 includes a generally cylindrical body that attaches to the arms 5206. The central body 5204 may include a central aperture 5208. Each arm 5206 includes a proximal portion 5210 integrally formed with, or connected to, the central body 5204, and a distal portion 5202 integrally formed with, or connected to, the proximal portion 5210. The features of the arms may be substantially similar to those features described in previous Examples.

FIG. 52 shows an example in which the torque transfer element 4608 and the rotatable member 5302 are formed as an integral unit, sharing the same axis of rotation. The torque transfer element 4608 is connected to the rotatable member 5302, where the torque transfer element 4608 is positioned to the front of the rotatable member 5302. The connection may be a rigid connection, or the torque transfer element 4608 may be integrally formed with the rotatable member 5302. A central aperture 5208 is located in the torque transfer element 4608 and rotatable member 5302.

The central body 5204 has a larger diameter than the rotatable member 5302, such that the central body 5204 has an overhanging portion relative to the rotatable member 5302, the overhanging portion defining an overhanging surface on which is positioned a first locking element 5212. The first locking element 5212 includes a plurality of teeth 4622 arranged on the overhanging surface. Each of the plurality of teeth 4622 are circularly arranged around the perimeter of the overhanging surface, are equally spaced and sized, and are uniformly slanted off-center.

The features of the first locking element 5212 and second locking element 4620 may be substantially similar to the first and second locking elements described in previous Examples.

The rotatable member 5302 is connected to the rear end of the torque transfer element 4608. The rotatable member 5302 is configured to drive one or more tension elements in a first direction or a second direction as the rotatable member 5302 is rotated in a first direction or a second direction, respectively. In this way, the effective length of the tension element can be shortened or extended by rotation of the rotatable member 5302. In this example, the tension elements and the rotatable member 5302 operate in a rack and pinion arrangement. In other examples, the tension element may operate in a filament and spool arrangement.

The torque transfer element 4608 and the rotatable member 5302 are formed as an integral unit, sharing the same axis of rotation. The torque transfer element 4608 is connected to the rotatable member 5302, where the torque transfer element 4608 positioned to the front of the rotatable member 5302. The connection may be a rigid connection, or the torque transfer element 4608 may be integrally formed with the rotatable member 5302. A central aperture 5208 is positioned in the torque transfer element 4608 and rotatable member 5302.

The rotatable member 5302 is mounted on the one or more tension elements. The rotatable member 5302 and the tension element can be positioned within the housing. One or more ends of the one or more tension element can connect to or be integrated into the headgear. The one or more tension elements can be flexible enough to wrap slightly around the rotatable member 5302 to provide more purchase between the tension element and the rotatable member 5302.

In this example, the rotatable member 5302 and the one or more tension elements interact as a pinion and rack, respectively. The interaction between the rotatable member and tension elements in this Example is substantially similar to that described in the Third Example. The rotatable member 5302 includes an annular body including a plurality of outwardly projecting spaced apart teeth 5304. As the rotatable member 5302 rotates, the teeth 5304 cause axial movement of the one or more racks. In this manner, the one or more racks can be used to adjust the tension on the headgear. In some examples, the rotatable member 5302 may have two or more annular bodies each including a plurality of outwardly projecting spaced apart teeth. Each annular body may be configured to receive one or more tension elements. In some examples, two or more annular bodies may have a different diameter.

Any suitable locking mechanism can be used to lock the position of the headgear, the one or more racks, the rotatable member 5302 and/or the actuator 4610. For instance, a pin or the like can be used to inhibit rotation of the rotatable member 534 and/or the actuator 4610. In some configurations, a friction brake, a clamping mechanism, a cammed brake member or the like can be used to inhibit movement of one or more of the headgear, the one or more racks, the rotatable member 5302 and/or the actuator 4610.

In some such configurations, a coil spring or other biasing member can urge the input device toward the limit associated with the smaller headgear size. As such, the headgear can expand but then automatically retract to the predetermined use size under the influence of the biasing member.

### Housing

As shown in FIG. 47 and FIG. 48, the housing 5502 includes a lower body 4604 and an upper body 4606 configured to mate together. The housing 5502 is generally arcuate in shape, and configured to be connected to or integrally formed with a strap for headgear. The housing shown in FIG. 46 to FIG. 48 is configured to be formed with a top strap for headgear, but the assembly could be configured as a rear strap or side strap.

The lower body 4604 and upper body 4606 interact by joining together at side edges to define an internal housing for retaining the rotatable member 5302 and one or more tension elements. Upper body 4606 includes a central opening 5102, and left and right side arms 4902 including a shape and curvature corresponding with the shape and curvature of the left and right side arms 4102 of the lower body 4604.

The upper body 4606 includes an opening 4614 on a middle portion thereof for receiving the rotatable member 5302. An upper side of the upper body 4606 is configured to rotatably engage with the actuator 4610, such that the actuator 4610 may rotate relative to the upper body 4606 when held in a fixed position.

The lower body 4604 includes a post 4616 for rotatably mounting the rotatable member 5302 at the central aperture 5208.

The left and right side arms 4618 on the lower body 4604 cooperate with the respective left and right side arms 4902 on the upper body 4606. The side arms 4902 of the upper body 4606 and the lower body 4604 define a left and right guide channel for directing the respective tension element along a path from the torque transfer element 4608 to the left and right side arms. The guide channels retain the tension elements along a path from the tension element apertures in the housing towards outer lateral ends of the side arms. Left and right guide channels are further defined into a front guide channel 4702 and a rear guide channel 4704 by one or more ridges 4706 disposed along a portion of the side arms on the lower body 4604. The one or more ridges 4706 act to keep the first and second tension elements separate from one another, for example as they move in opposite directions from each other as they are driven in first and second directions.

One or more ridges 4802 may be positioned on an inner side of the upper body 4606 to cooperate with the ridges 4706 on the lower body 4604. For example, FIG. 47 shows left and right side ridges 4706 on the lower body 4604 which are overlapped by the ridges 4802 (see FIG. 48) on the upper body 4606. The ridges thus have a function of interlocking the upper body 4606 and the lower body 4604 in addition to defining the guide channels for the tension elements. The upper and lower body may interlock by a snap fit connection. In this example, the guide channels taper outwardly from a central part of the housing 5502 such that the tension element pathway expands as it extends outwardly from the rotatable member 5302.

The upper body 4606 includes an opening 4614 in a central portion thereof surrounded by a second locking element 4620. The second locking element 4620 is shaped and sized to complementarily match the shape and size of the first locking element 5212. In this regard, the second locking element 4620 includes a plurality of upwardly projecting teeth 4622 arranged on a top side of the upper body 4606. The teeth 4622 are circularly arranged around the perimeter of the opening 4614, are equally spaced and sized, and are uniformly slanted off-center. The angle of the off-center slant is reciprocal/inverse to the angle of the off-center slant of the teeth in the first locking element 5212.

The interaction between the first locking element 5212 and the second locking element 4620 allows incremental tightening of the tension elements. Rotation of the actuator communicates torque to the rotatable member 5302 via the torque transfer element 4608. As the actuator 4610 is rotated in the tightening direction, the teeth of the first locking element 5212 rotate over complementarily shaped teeth on the second locking element 4620.

The features of the first and second locking elements may be substantially similar to the first and second locking elements described in the Examples above.

### Tension Elements

A patient interface assembly including first and second tension elements 5704 is shown in FIG. 57. Each tension element 5704 includes a rack portion 5714 located along an end portion of the tension element including a series of teeth. The tension elements are formed of a rigid or semi rigid material. The rack portion 5714 may be sufficiently flexible to be driven about the rotatable member 5302. Preferably, the tension element 5704 is sufficiently stiff along its length to be pushed out of the adjustment assembly and along pathways 5712 in headgear 5702 by the rotation of the rotatable member without significant bunching. Part of the tension element may be integrally formed with headgear straps, such as a top strap. Alternatively, the tension element part including the rack portion 5714 may be located in the housing 5502. The tension element 5704 may extend along a pathway 5712 in the headgear straps to a distal end of the side straps 5710.

The housing 5502 is configured to receive first and second tension elements. The first and second tension elements are configured to engage the rotatable member 5302, such that the first tension element is driven across an upper side of the rotatable member 5302 in a first direction and the second tension element is driven across a lower side of the rotatable member 5302 in the opposite direction. Rotation of the rotatable member 5302 thus drives the first and second tension elements in opposing directions. When the tension elements are connected to the headgear, rotation of the rotatable member 5302 in either direction thus effects a respective tightening or loosening of the headgear.

### Biasing Member

As with the above Examples, axial movement of the torque transfer element 4608 in the actuator interior is in part controlled by the axial biasing of the torque transfer element 4608 downwardly (towards the bottom wall of the actuator 4610), that is, axial biasing of the torque transfer element 4608 towards the rear end of the ramp 5014. Axial biasing can be produced with springs or the use of other resilient members that exert an axial urging force on the torque transfer element 4608.

Accordingly, biasing member 4626 may be positioned in the interior of the actuator 4610 between the covering cap 4612 and the torque transfer element 4608 to exert a rearward biasing force on the torque transfer element 4608. However, biasing member 4626 may be positioned in other parts of the adjustment assembly 4602 and still provide the biasing force. For example, the biasing member 4626 may be located to the rear of the rotatable member 5302 and/or torque transfer element 4608.

### Headgear

Headgear may be provided including connection means for engaging with the adjustment assembly 4602. Alternatively, the headgear may be integrally formed with the adjustment assembly 4602. For example, the adjustment assembly 4602 may be integrally formed with a top strap, rear strap, or side strap of the headgear.

Headgear 5702 includes side straps 5710, a top strap 5708 and a rear strap 5706, and one or more pathways 5712 for receiving tension elements 5704.

FIG. 57 shows adjustment assembly 4602 incorporated into a top strap 5708 of a headgear 5702. lower body 4604 may be attached or molded onto a headgear strap, such as top strap 5708.

### FIFTH EXAMPLE

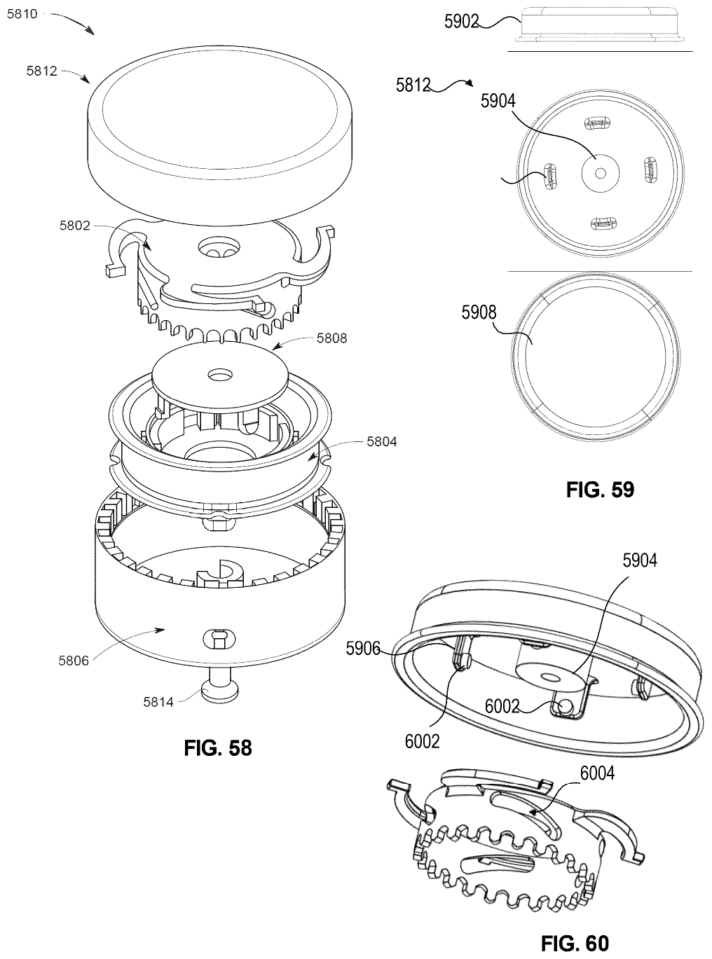

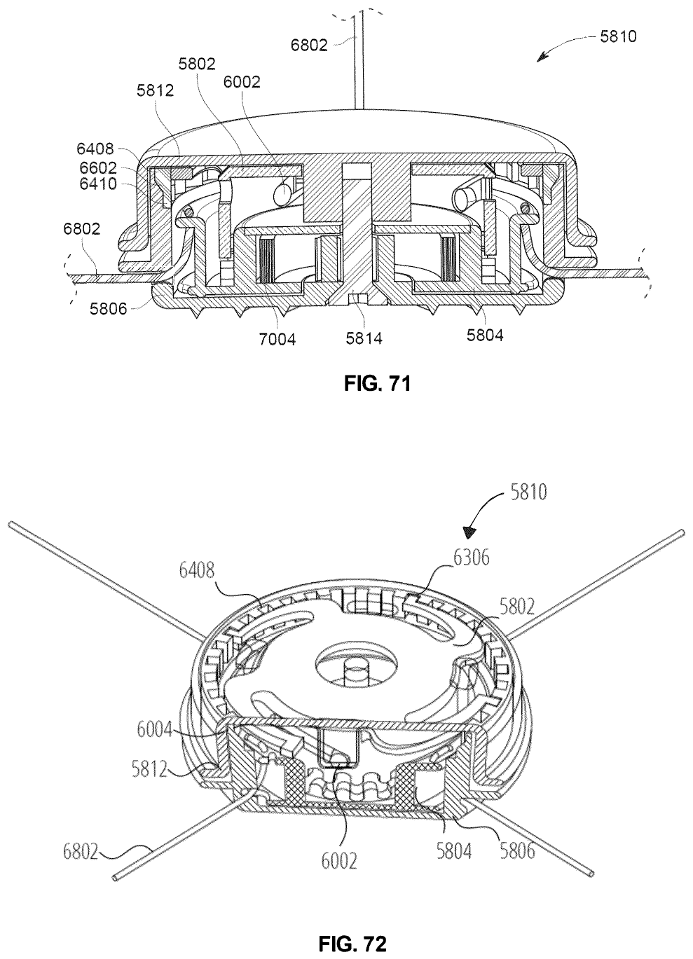

With reference to FIG. 58 to FIG. 71, there is provided a Fifth Example of an adjustment assembly 5810. The adjustment assembly 5810 may include an actuator 208, torque transfer element 5802, rotatable member 5804, and base 5806.

### Actuator

With particular reference to FIG. 59, the assembly includes an actuator 5812 configured to be rotated by a user, including a top wall 5908, side wall 5902, and an interior defined by the top wall 5908 and side wall 5902. A circular mount 5904 extends from the center of the top wall 5908 into the interior, the mount 5904 including a recess in its center. A plurality of posts 5906 extend from the top wall 5908 into the interior. Each of the plurality of posts 5906 are ideally equally spaced apart from other posts 5906, equally spaced apart from the circular mount 5904, and spaced apart from the side wall 5902. A protrusion 6002 extends substantially perpendicularly from the side of the post 5906 at a distal end thereof.

### Torque Transfer Element

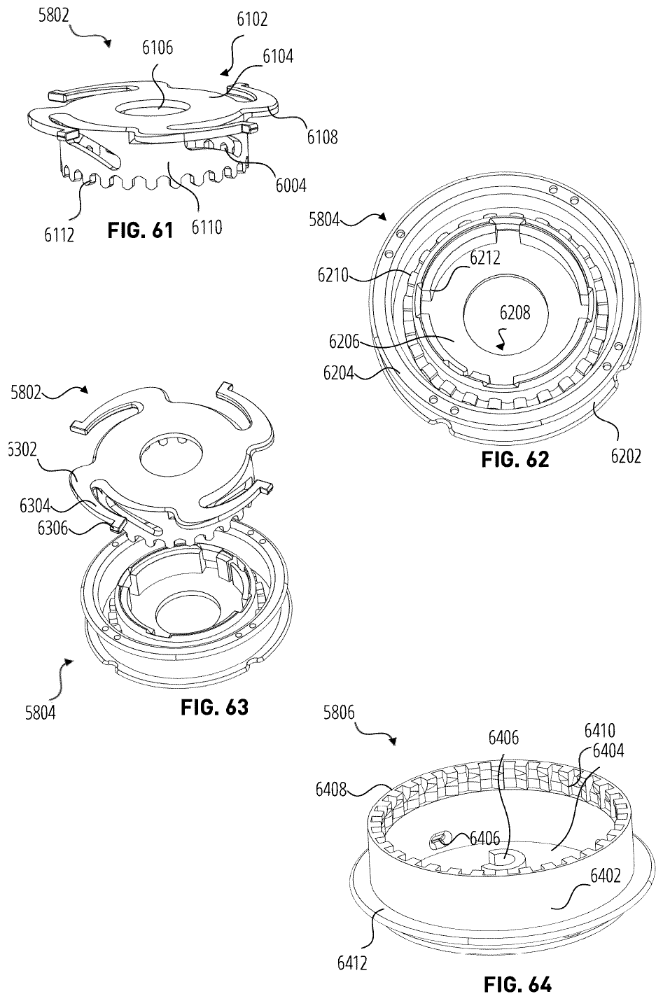

The assembly further includes a rotatable torque transfer element 5802 for receiving torque from the actuator 5812. With particular reference to FIG. 60 and FIG. 61, the torque transfer element 5802 is configured to be received in the interior of the actuator 5812. The torque transfer element 5802 includes a central body 6102 including a top wall 6104 with a central opening 6106, and one or more arms 6108 extending radially from an upper portion of the central body 6102. The torque transfer element 5802 may include any number of arms, for example, between one and eight, although as shown in FIG. 61, in one example the torque transfer element 5802 includes four arms. The central opening 6106 is shaped to receive the mount 5904 of the actuator 5812 therethrough. Each arm 6108 may have substantially the same features as described for the arms in the torque transfer elements of earlier Examples described herein. For example, each arm 6108 includes a proximal portion 6302 integrally formed with, or rigidly connected to, the central body 6102, a distal portion 6304 integrally formed with, or rigidly connected to, the proximal portion 6302, and a distal protrusion 6306 extending radially outwardly from the distal portion 6304 and the central body 6102.

A side wall 6110 of the torque transfer element 5802 extends perpendicularly relative to the top wall 6104, defining a cylindrical body. A locking element is positioned on a lower end of the side wall 6110, configured to engage with a complementary locking element located on the rotatable member 5804. The locking element includes a plurality of downwardly projecting teeth 6112. The locking element may include between 10 and 40 teeth 6112, and as shown in FIG. 61, between about 20 and 26 teeth 6112. Each of the plurality of teeth 6112 are circularly arranged about the lower end of the side wall 6110, and are substantially equally spaced and sized. The teeth 6112 may each be symmetric. In an example, each of the plurality of teeth 6112 may be oriented in a substantially perpendicular arrangement relative to the bottom end of the side wall 6110, such that the teeth 6112 are not angled off-center, and no directional bias is imparted to the locking element. The plurality of teeth 6112 are configured to intermesh with a plurality of teeth on a complementary locking element positioned on the rotatable member 5804. Each of the plurality of teeth 6112 are complementarily shaped such that the tips of each tooth on one side engaging with a groove between teeth 6112 on the other side.

One or more elongate apertures 6004 are positioned along portions of the side wall 6110. The elongate apertures 6004 are sloped from a lower part to an upper part of the side wall 6110 along a distance of the side wall circumference. The elongate apertures 6004 are configured to slidably engage with the protrusions 6002 of the actuator posts 5906.

In use, rotation of the actuator 5812 causes the post protrusions 6002 to slide along the sloped elongate apertures 6004 and abut at an end thereof. Further rotation of the actuator 5812 in the same direction directly transfers torque from the actuator 5812 to the torque transfer element 5802. As the elongate aperture is sloped from an upper part to a lower part of the side wall 6110, the rotation of the actuator 5812 imparts an axial translation of the torque transfer element 5802 relative to the actuator 5812.

### Rotatable Member

The assembly further includes a rotatable member 5804 for winding, storing, and unwinding the tension element 6802, where the rotatable member 5804 is reversibly engageable with the torque transfer element 5802. With particular reference to FIG. 62 and FIG. 63, the rotatable member 5804 includes an outer wall 6202 including upper and lower outwardly extending rims 6204, a lower wall 6206 extending perpendicularly inwardly from the outer wall 6202, and a central opening 6208 in the lower wall 6206.

An interior of the rotatable member 5804 is defined by the inner sides of the outer wall 6202 and lower wall 6206. A plurality of upwardly facing teeth 6210 are positioned on the inner side of the lower wall 6206, configured to reversibly interlock with the downwardly facing teeth 6112 on the torque transfer element 5802. An inner wall 6212 extends upwardly from the lower wall 6206 on the inner side of the plurality of teeth 6210. The outer wall 6202, inner wall 6212 and the upwardly facing teeth 6210 define an annular trough in the rotatable member 5804 into which the side wall 5902 of the torque transfer element 5802 can be nested. Reversible axial translation of the torque transfer element 5802 driven by the actuator 5812 causes the reversible engagement and disengagement of the teeth 6112 on the torque transfer element 5802 and teeth 6210 on the rotatable member. For example, rotation of the actuator 5812 in a tightening direction drives the post protrusions 6002 towards the upper end of the elongate apertures 6004 and thus the axial movement of the torque transfer element 5802 towards the rotatable member 5804. Rotation of the actuator 5812 in an untightening direction drives the post protrusions 6002 in the opposite direction towards the lower end of the elongate apertures 6004 and thus causes an axial retraction of the torque transfer element 5802 away from the rotatable member 5804 and further into the interior of the actuator 5812. The torque transfer element 5802 and rotatable member 5804 are thus configured such that the axial movement produces a reversible engagement and disengagement of the teeth on the torque transfer element 5802 and the rotatable member 5804.

The outer side of the outer wall 6202 is configured to wind, store, and unwind one or more tension elements. The upper and lower rims 6204 assist in retaining the tension elements on the rotatable member 5804.

### Base

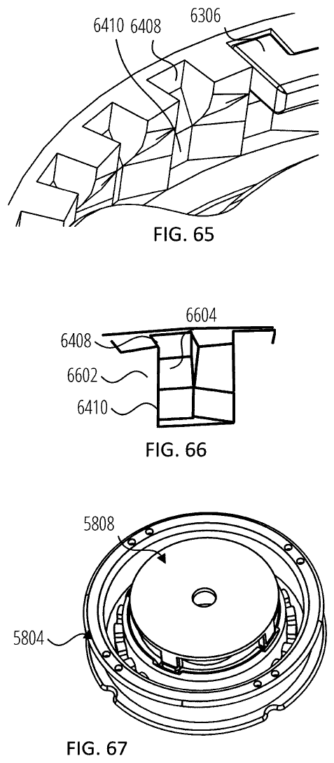

With particular reference to FIG. 64, FIG. 65 and FIG. 66, the assembly includes a base 5806 comprising a side wall 6402 and a lower wall 6404 defining a base interior that is configured to receive the rotatable member 5804 and the torque transfer element 5802. One or more openings 6406 in the side wall 6402 are provided to direct the tension element 6802 between the rotatable member 5804 and the exterior of the base 5806. A substantially vertical post 6902 is located in the center of the lower wall 6404, which is configured to extend through and mount the central opening of lower wall of the rotatable member 5804 so that it may rotate about the post 6902. The post 6902 may be cylindrical or have a C-shaped cross-section, and includes a central vertical recess.

The adjustment assembly 5810 is configured to connect to a patient interface or headgear via the base 5806, which may include fasteners or connectors to engage with the headgear or patient interface. For example, FIG. 64 shows the base including an extended rim 6412 on the exterior side of the outer wall which can engage with headgear material.

Around the inner perimeter of an upper part of the base side wall 6402 there is provided an upper set 6408 and a lower set 6410 of indentations each configured to interact with the distal protrusions 6306 of the torque transfer element arms 6108. The upper and lower sets of indentations are configured such that the distal protrusion 6306 can move axially between the upper and lower sets, and thus between the engaged and disengaged configuration.

The torque transfer element 5802 is engaged with the upper set 6408 of indentations when in its disengaged configuration. The upper set 6408 of indentations have steeply angled lateral sides (e.g., approximately at right angles to the inner wall) which are configured such that the distal protrusions 6306 of the torque transfer element 5802 are rotationally locked with the base 5806, and such that the torque transfer element 5802 is prevented from rotating relative to the base 5806.

The torque transfer element 5802 is engaged with the lower set 6410 of indentations when in its engaged configuration. The depth of each of the lower indentations is less than the depth of the upper indentations. The arms 6108 of the torque transfer element 5802 may therefore be deformed inwardly as the torque transfer element 5802 is translated from the upper set 6408 of indentations to the lower set 6410. Each of the lower indentations include a laterally sloping face on one side of the indentation, which is configured to enable the distal protrusions 6306 to slip between contiguous lower indentations. Slipping of the distal protrusions 6306 is achieved by inward deformation of the arms 6108 as they are urged inwardly during rotation of the distal protrusion 6306 against the sloped face of the indentation. The opposing side of the indentation is more steeply angled (and may be substantially similarly angled to the upper set of indentations), such that the distal protrusions may be directionally biased in one rotational direction, being the tightening direction.

As best shown in FIG. 66, situated between the upper and lower sets of indentations is a transition zone 6602 configured to guide the distal protrusions 6306 and to permit a smooth translation of the torque transfer element 5802 between upper and lower sets of indentations. The transition zone 6602 includes a radial ramp 6604 portion that spans the difference in radial depth between the upper and lower sets of indentations.

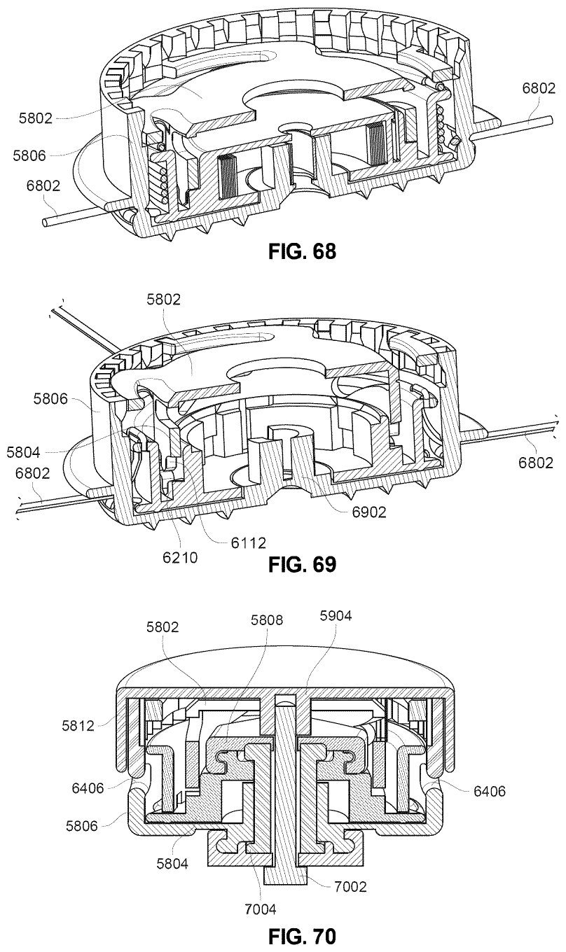

With particular reference to FIG. 68, in the engaged configuration, the teeth 6112 on the torque transfer element 5802 are intermeshed with the teeth 6210 on the rotatable member 5804, and the distal protrusions 6306 sit in the lower set 6410 of indentations on the base 5806. Rotation of the actuator 5812 in the tightening direction causes the rotation of the torque transfer element 5802 relative to the base 5806, which is enabled by the inward deformation of the arms 6108 about the sloped surfaces of the lower indentations. Rotation of the torque transfer element is communicated directly to the rotatable member due to the intermeshing of the respective downwardly and upwardly facing teeth, which in turn causes the tightening of the tension element about the rotatable member. The rotatable member 5804 incrementally winds more tension element and tightens the headgear in a controlled manner.

With particular reference to FIG. 69, rotation of the actuator 5812 in the untightening direction causes the assembly to shift to the disengaged configuration, in which the teeth 6112 on the torque transfer element 5802 are axially translated away from the teeth 6210 on the rotatable member 5804, and the distal protrusions 6306 shift from engagement with the lower set 6410 of indentations to the upper set 6408 of indentations via the transition zone 6602. In this configuration, the torque transfer element 5802 and base 5806 are rotationally locked together, and the rotatable member 5804 is able to rotate freely in the base interior, thus allowing the tension elements to be wound and/or unwound from the rotatable member 5804 in response to external forces from the user (e.g., the user pulling on the tension elements or headgear straps), or from forces from a biasing member.

### Cover and Biasing Member

As shown in FIG. 67, a cover 5808 is connected to the rotatable member 5804, and positioned between the torque transfer element 5802 and the rotatable member 5804. The cover 5808 has a circular top surface with a central aperture, and one or more legs that engage with the inner wall 6212 of the rotatable member 5804. The legs engage with the rotatable member 5804 to hold the cover 5808 in place. The engagement may be reversible.

In one example, the pin 5814 connecting the base to the actuator 5812 may comprise a rod 7002, may be constructed of an elastic or resilient material that may bias the actuator 5812 towards the base 5806 and thus bias the adjustment assembly 5810 into the engaged configuration. The rod 7002 may be a resilient piece of silicone which can be stretched as the adjustment assembly 5810 is moved into the disengaged configuration. The rod 7002 may pass through central openings in the rotatable member 5804, and torque transfer element 5802 and engage within the recess of the mount 5904 of the actuator 5812.

Alternative biasing members may be used. For example, a coil spring may be provided, which surrounds and is supported by the base post and extends through the central opening 6208 of the rotatable member 5804 to the lower surface of the cover 5808. The coil spring may be connected to the base 5806 and cover 5808 at each end such that the rotatable member 5804 is rotationally biased in the tightening direction.

The adjustment assembly may further comprise a torsional biasing member 7004 configured to bias the rotational member 5804. FIG. 70 shows torsional biasing member 7004 positioned about the rod 7002 and extending between the base 5806 and rotatable member 5804. Torsional biasing member 7004 may be a tube or rod of resilient or elastic material, such as a silicone polymer. A lower end of the torsional biasing member connects to or through the base. An upper end of the torsional biasing member connects to the rotatable member. Preferably, torsional biasing member 7004 biases the rotatable member 5804 in the tightening direction so that, in use, the adjustment assembly 5810 is biased to at least partially retract the tension elements 6802 about the rotatable member 5804. However, torsional biasing member 7004 may be biased towards other states, such as to at least partially extend the tension elements 6802.

FIG. 72 to FIG. 75 show the engaging and tightening operation of the adjustment assembly 5810 of the Fifth Example. FIG. 72 shows the adjustment assembly 5810 with four tension elements 6802 each extending from the adjustment assembly 5810 body substantially equidistantly apart. The torque transfer element 5802 is in the disengaged configuration, so the distal protrusions 6306 of its arms 6108 sit in the upper set 6408 of indentations on the base, and the locking element on the torque transfer element 5802 is axially translated away from the locking element on the rotatable member 5804.

The post protrusions 6002 on the actuator 5812 (actuator 5812 is shown in FIG. 72 to FIG. 78 as a transparent outline, so as to show inner parts) are located at a lower end of the elongate apertures 6004 on the torque transfer element 5802. The rotatable member 5804 is able to rotate independently of the torque transfer element 5802 (and by extension, the actuator 5812) in the base interior. Each tension element 6802 (two of which are shown) is shown fully extended from the rotatable member.

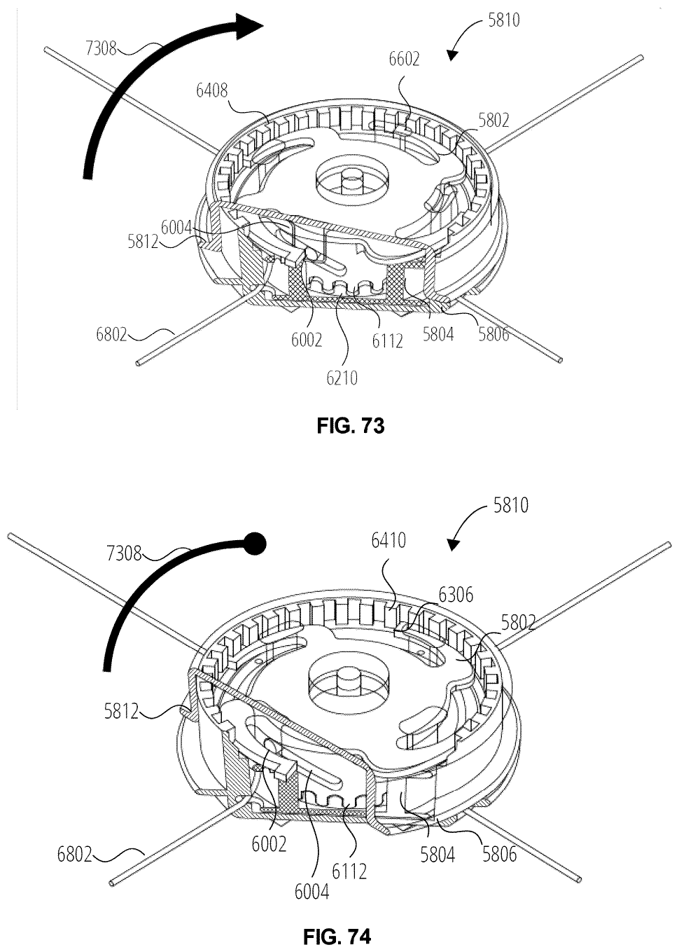

FIG. 73 shows the adjustment assembly 5810 in an intermediate position between the disengaged configuration shown in FIG. 72 and an engaged configuration, as a result of a rotation of the actuator 5812 in the tightening direction shown by the curved arrow 7308 in FIG. 73 (the clockwise direction). Rotation of the actuator 5812 causes its post protrusions 6002 to slide along the elongate apertures 6004 on the torque transfer element 5802 and drive the torque transfer element 5802 axially towards the rotatable member 5804. The teeth 6112 of the torque transfer element 5802 partially intermesh with the teeth 6210 on the rotatable member 5804, and the distal protrusions 6306 on the torque transfer element 5802 shift axially from the upper set 6408 of indentations into the transition zone 6602. Accompanying the axial shift is a radial inward deformation of the arms 6108 due to a difference in radial distance of the upper set and lower set of indentations.

Further clockwise rotation 7308 of the actuator in FIG. 73 drives the torque transfer element 5802 into the engaged configuration, which is shown in FIG. 74 and in which the movement of the actuator driving the adjustment assembly into the engaged configuration is indicated by the curved line and circle in FIG. 74. The post protrusions 6002 continue to slide along the elongate apertures 6004 and cause further axial movement of the torque transfer element 5802 so that its teeth 6112 further intermesh with the teeth 6210 on the rotatable member 5804. The distal protrusions 6306 shift from the transition zone 6602 into the lower set 6410 of indentations.

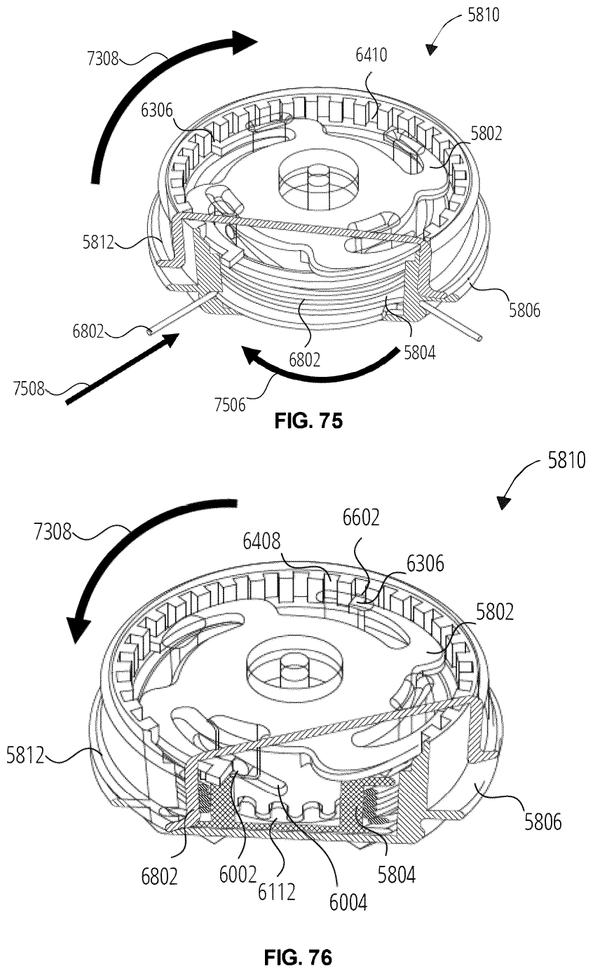

In the engaged configuration shown in FIG. 75, the actuator 5812 and rotatable member 5804 are rotationally coupled such that further rotation 7308 of the actuator 5812 in the tightening direction causes rotation 7506 of the rotatable member 5804 and retraction 7508 of tension element 6802 about the rotatable member 5804. FIG. 75 shows the rotatable member 5804 with a length of tension element 6802 wound about an annular channel in its outer wall as a result of rotation of the rotatable member 5804, retraction of the tension elements 6802 and the resulting shortening of the effective length of the tension elements 6802 outside of the adjustment assembly 5810 (which, when worn, corresponds to a tightening of the headgear about the user's head).

FIG. 76 to FIG. 78 show the disengaging and untightening operation of the adjustment assembly. FIG. 76 shows the initial stage of disengagement and untightening, such as to loosen or doff the assembly. The user rotates 7308 the actuator 5812 in the untightening direction (shown in FIG. 76 as an anticlockwise direction). The post protrusions 6002 slide along the elongate apertures 6004 to drive the torque transfer element 5802 axially away from the rotatable member 5804, thus causing the teeth 6112 to begin to disengage from the teeth 6210 of the rotatable member, and the distal protrusions 6306 to move into the transition zone 6602.

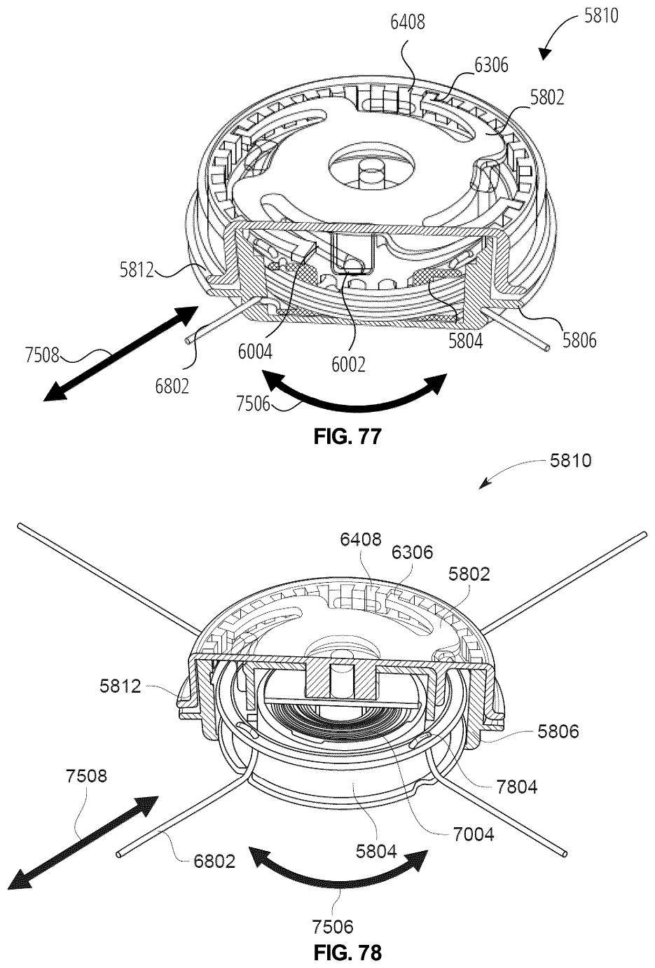

FIG. 77 and FIG. 78 shows the torque transfer element 5802 fully disengaged from the rotatable member 5804, such that the rotatable member 5804 may rotate 7506 independently of the torque transfer element 5802. The tension elements 6802 may be unwound 7508 from the rotatable member 5804 by a user manually extending the tension elements 6802 (e.g., by pulling on the tension elements or the headgear straps that contain them). FIG. 78 shows the tension elements fully unwound from the rotatable member. The distal protrusions 6306 are positioned in the upper lower set 6410 of indentations, which prevents further rotation of the actuator 5812 in the untightening direction. Each tension element 6802 is connected to the rotatable member 5804 at a tension element attachment point 7804, so it does not completely come away from or slip around the rotatable member 5804.

FIG. 78 shows a torsional biasing member 7004 biasing the rotatable member 5804 towards the tightening direction (i.e., clockwise in this example). The torsional biasing member 7004 may be any element suitable for storing and releasing tension about an axis. The torsional biasing member 7004 is shown as a spiral spring, but other suitable components could be used, such as other torsion springs such as coil springs or elastic materials. The torsional biasing member 7004 is located in a central recess of the rotatable tension element 6802 and connected at a first end to the rotatable member 5804 and connected at a second end to the base 5806. The untightening force on the rotatable tension element 6802 exerted by the user pulling on the tension elements 6802 builds torsional tension in the torsional biasing member 7004. When the unwinding force is released, the torsional biasing member 7004 returns to its relaxed state by rotating the rotatable element in the tightening direction (clockwise) and thus retracts at least a portion of the tension element(s).

Alternatively, the biasing direction of the torsional biasing member 7004 can be reversed so that it builds torsional tension in the tightening direction and drives the rotatable member 5804 in the untightening direction. This arrangement may be particularly useful where the tension elements 6802 are sufficiently stiff to be unspooled and pushed out of the adjustment assembly by the rotation of the rotatable member without bunching. For example, the tension elements 6802 may be filaments that may be wound about the rotatable member 5804 but also stiff enough to transfer compressive force through the tension element 6802.

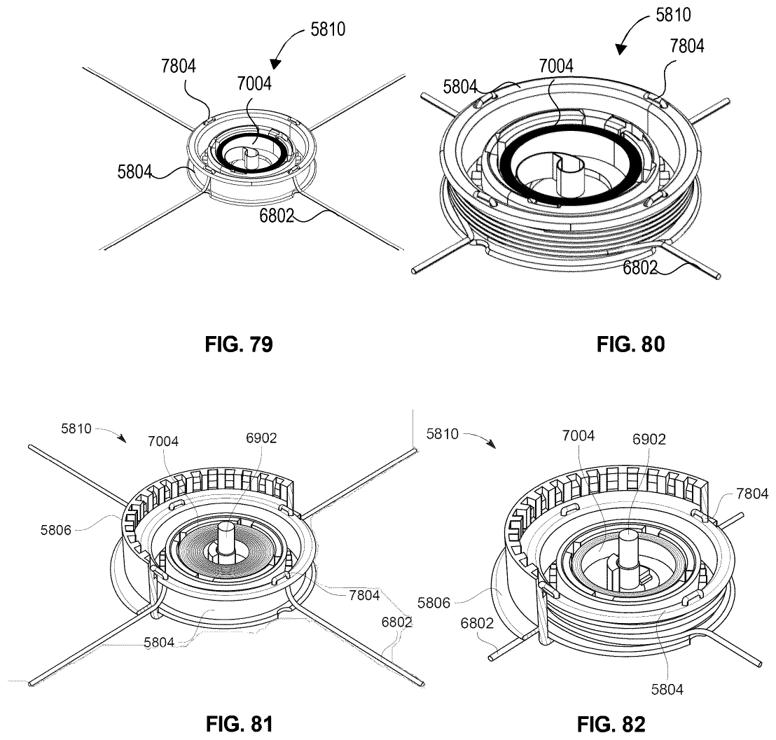

FIG. 79 to FIG. 85 show parts of the adjustment assembly 5810 in a progressive assembly. FIG. 79 and FIG. 80 show the rotatable member 5804, torsional biasing member 7004 (a spiral spring) positioned around the vertical post 6902, and tension elements 6802 in extended (FIG. 79) and retracted (FIG. 80) states, respectively.

FIG. 81 and FIG. 82 show the rotatable member 5804 positioned in the base 5806 (shown in cut-away) in extended and retracted states of the tension elements 6802, respectively. The spiral spring is in a torsionally tightened state when the tension elements 6802 are fully extended from the rotatable member 5804, and in a relaxed state when the tension elements 6802 are at least partially retracted about the rotatable member 5804. Thus, the spiral spring biases the rotatable member 5804 towards an at least partially retracted state. Although as noted above, the torsional bias of the spiral spring may be reversed so that the biasing direction is towards an at least partially extended state.

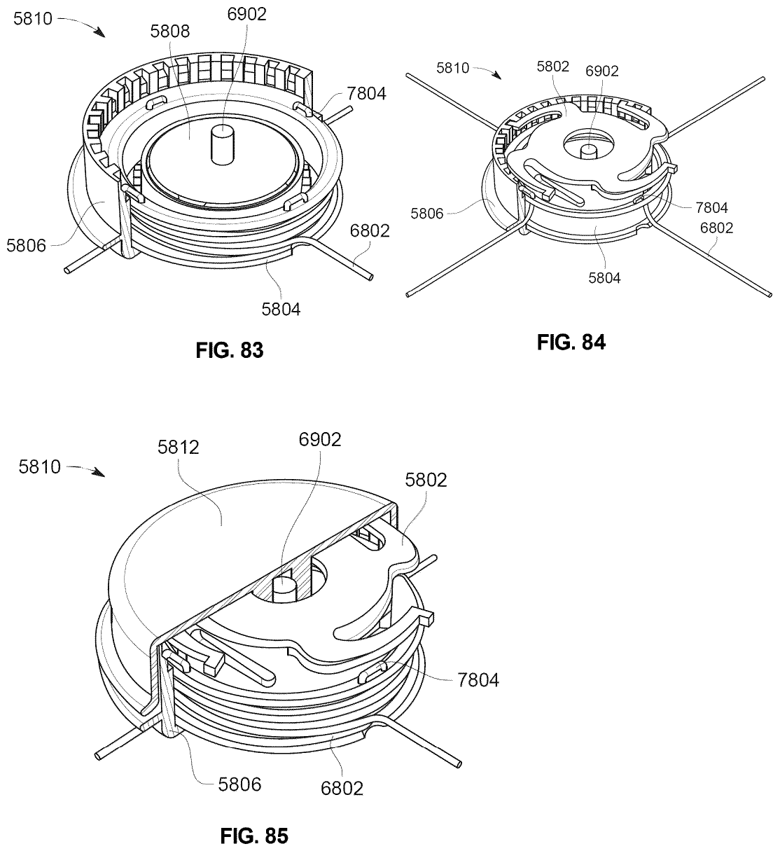

FIG. 83 shows the torsional biasing member 7004 and rotatable member 5804 fitted with a cover 5808. The cover 5808 acts to contain the torsional biasing member 7004 within the central recess of the rotatable member 5804 and prevent it from interfering with the torque transfer element 5802. FIG. 84 shows the parts of FIG. 83 fitted with the torque transfer element 5802 (positioned in the disengaged configuration, with the tension elements in the extended state). FIG. 85 shows the parts of FIG. 84 fitted with the actuator 5812.

The adjustment assembly 5810 of the Fifth Example described above may be modified to include features that prevent a user from overtightening the adjustment assembly, which can cause discomfort in wearing a patient interface assembly. Generally, the prevention of overtightening may be achieved using one or more features that decouple the rotation of the actuator from rotation of the rotatable member above a torque threshold on the rotatable member. The torque threshold may be predetermined to correspond to a maximum tightness of the patient interface assembly on a user's face. The adjustment assembly may comprise a torque limiting mechanism comprising an input member configured to receive torque from a user and an output member rotatably coupled with the rotatable member, wherein the input member and output member are rotationally coupled up to a torque threshold, and rotationally decoupled above the torque threshold.

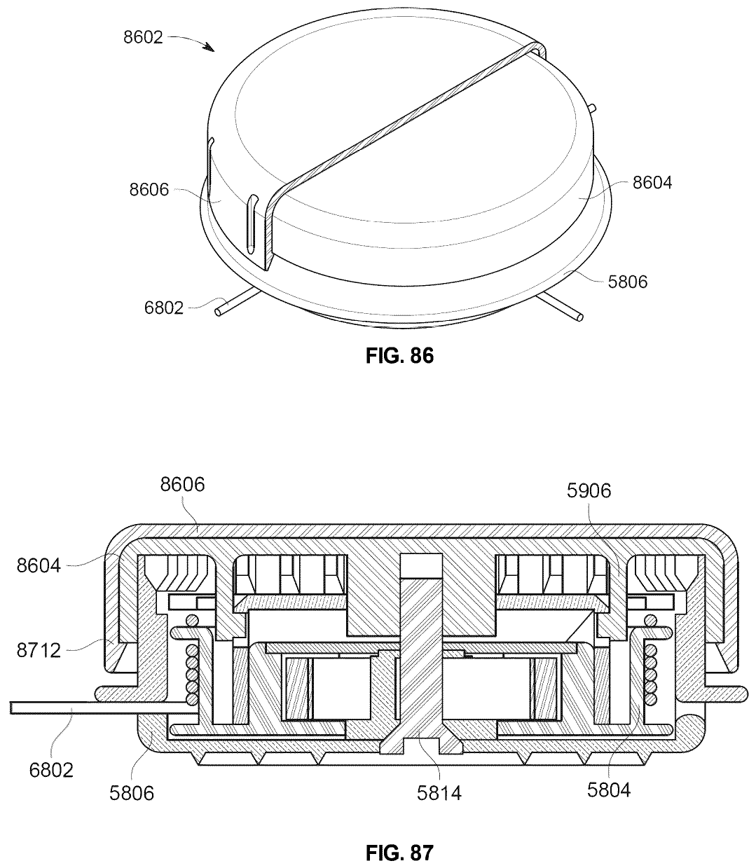

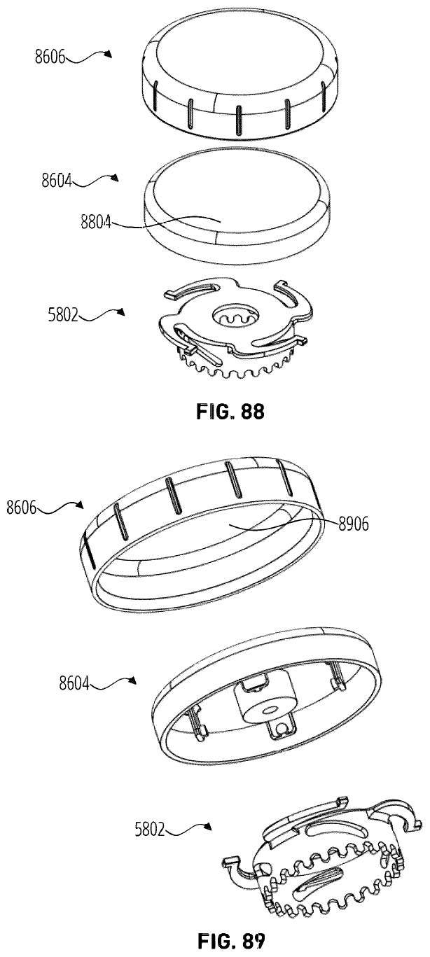

FIG. 86 to FIG. 89 show an example of the adjustment assembly 5810 for a patient interface assembly comprising a modified actuator 8602 that is configured to prevent a user from overtightening the patient interface assembly. The actuator 8602 comprises an inner member 8604 and an outer member 8606 nested together and a frictional contact between smooth planar interfacing surfaces (e.g., at least part of the outer surface 8804 of the inner member 8604 is in frictional contact with at least part of the smooth inner surface 8906 of the outer member 8606). The outer member receives a torque input, e.g., from the user by directly rotating the outer member. The inner member 8604 delivers the torque output to the torque transfer element. The outer member 8606 transfers torque to the inner member 8604 via the interfacing surfaces. The outer member 8606 may be configured to be directly actuated by a user, and the inner member 8604 may be configured substantially as the actuator 5812 described above in respect of FIG. 59 and FIG. 60.

The interfacing surfaces may have a predetermined frictional engagement, such that the inner member 8604 and outer member 8606 are rotationally coupled at rotational forces up to a torque threshold, and the inner member 8604 and outer member 8606 are rotationally decoupled above the torque threshold (so the interfacing surfaces of the outer member 8606 and inner member 8604 slide over each other).

The torque threshold may be better controlled if the compressive forces between the interfacing surfaces are controlled. Outer member 8606 and inner member 8604 may be retained in a controlled interfacing contact by lip 8712 (shown in FIG. 87 as an overlapping lip on outer member). For example, the outer member 8606 is preferably constructed of sufficiently rigid material to resist deformation by a user's grasping or squeezing action, e.g., during rotation by a user's fingers. The interfacing surfaces may be biased together with a biasing member.

The interfacing surfaces may comprise a material for achieving the desired frictional contact. For example, the interfacing surfaces on one or both of the inner member and outer member may comprise a silicone film.

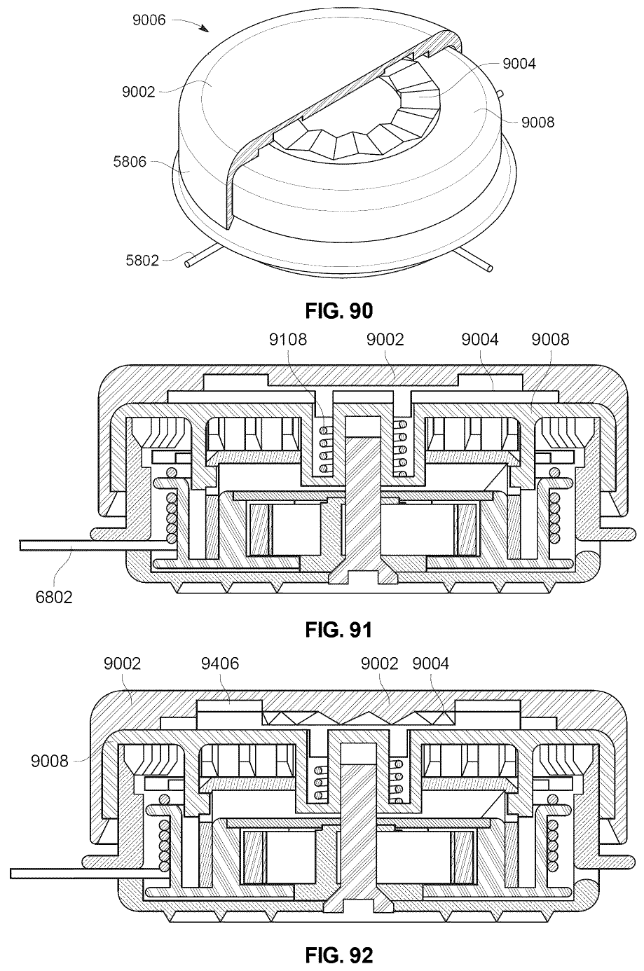

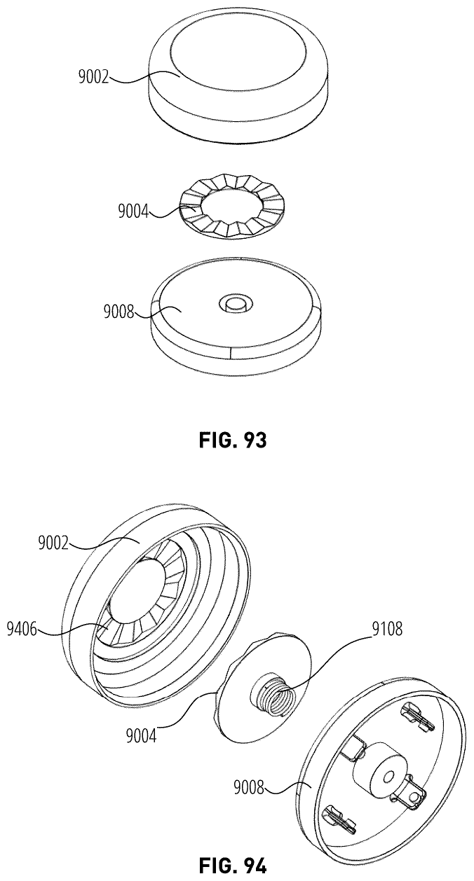

FIG. 90 to FIG. 94 show an alternative example of the modified actuator shown in FIG. 86 to FIG. 89, in which the interfacing surfaces comprise non-planar formations to increase the traction between the torque input and rotational output or provide some tactile or acoustic feedback to a user. Actuator 9006 comprises radially ridged interfacing surfaces between the inner member 9008 and outer member 9002. The example shown in FIG. 90 to FIG. 94 comprise 12 intermeshing ridges, but other numbers of ridges would be suitable, for example, between six and 24 ridges.

At least one of the interfacing surfaces may be biased towards the other to modulate the frictional engagement. In FIG. 90 to FIG. 94, the first ridged surface 9004 located on the inner member 9008 is biased towards the ridged surface on the outer member with biasing member 9108. The inner member 9008 comprises a central recess to receive a portion of the biasing member 9108 on the underside of the first ridged surface 9004, in part to allow the ridged surface to rest flat against the inner member 9008. As the outer member is rotated, the interfacing surfaces are coupled and torque is transmitted to the inner member. As shown in FIG. 91 and FIG. 92, where the torque on the interfacing surfaces exceeds a maximum torque, the interfacing surfaces lose traction and slip over each other. Where the interfacing surfaces are non-planar, the loss of traction is accompanied by an axial movement, such as the outer member moving away or upward from the inner member (FIG. 92), which may be resisted by the biasing member 9108.

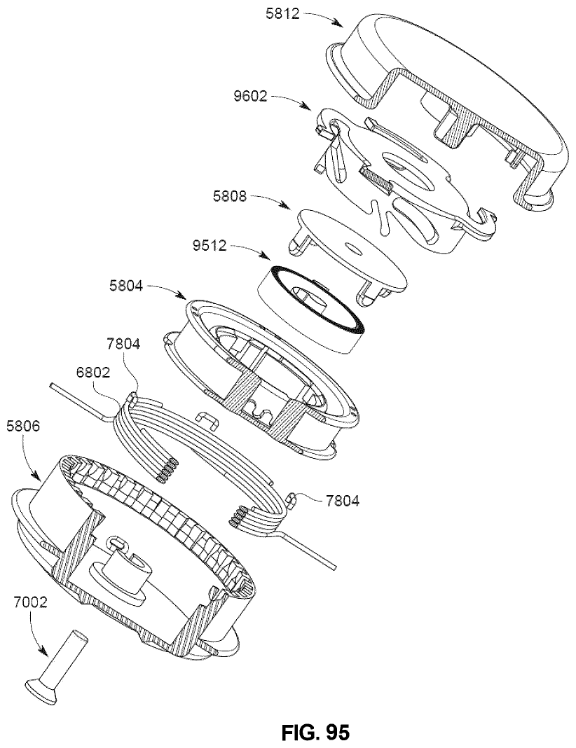

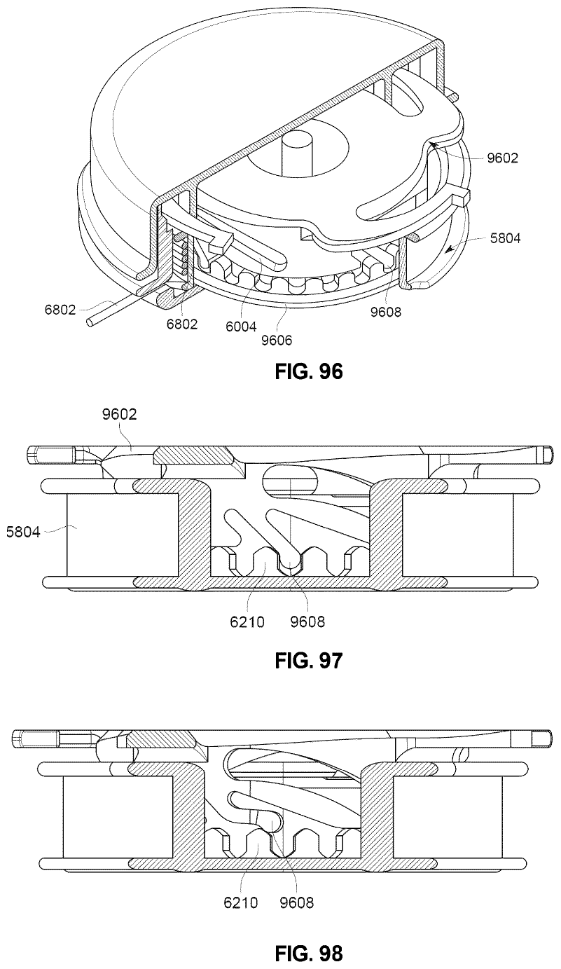

An alternative example of the torque transfer element in the Fifth Example is shown in FIG. 95 to FIG. 99. In this example, torque transfer element 9602 comprises one or more pawls 9608 connected to or integrally formed with the side walls of the torque transfer element (replacing the plurality of teeth 6112 in the example shown in FIG. 58 to FIG. 71). The example shown has four pawls 9608 substantially equidistantly spaced apart, although the number may be varied. The one or more pawls 9608 comprise a projection 9704 extending diagonally relative to the axial direction configured to engage with the complementary locking element on the rotatable member 5804. For example, the free end of the pawl 9608 may be configured to intermesh with the teeth 6210 in the complementary locking element on the rotatable member 5804 when the adjustment assembly is in the engaged configuration.

The one or more pawls 9608 may be constructed of a resilient material so it may deform about the rotatable member's locking element (e.g., the teeth 6210). The resilience of the pawl 9608 may be tuned so that it deforms about the rotatable member's locking element at a particular torque threshold. As the actuator is rotated in the tightening direction, the torque transfer element 9602 transfers the tightening rotational force onto the rotatable member 9604 (via the pawl 9608) which rotates and retracts the tension element 6802. At a particular tightness corresponding to the torque threshold, the pawl 9608 deforms and slips over the locking element on the rotatable member, preventing further tightening (see FIG. 98). The torque threshold may be predetermined to correspond to a particular tightness of the patient interface on a user's face.

The resilience and the angle of the pawl 9608 relative to the rotatable member's teeth may be tuned to resist rotational forces from the rotatable member 9604 in the untightening direction, at least at forces during normal use.

The deformation of the pawl 9608 over the rotatable member 5804 may be associated with audible or tactile feedback to the user, caused by the motion of the pawl 9608 slipping over the locking element on the rotatable member 5804, e.g., to indicate that the torque threshold has been reached.

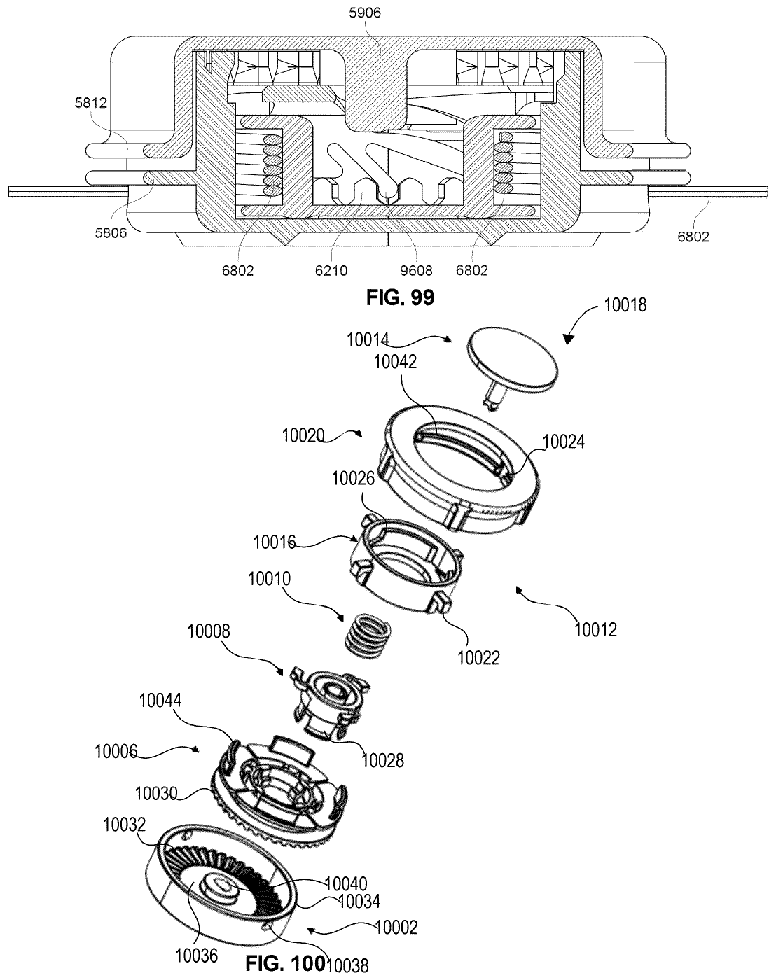

FIG. 97 and FIG. 99 show the example with a pawl 9608 intermeshed with the teeth of the rotatable member 5804. FIG. 98 shows the deformation of the pawl 9608 as the torque transfer element 9602 is rotated. FIG. 95 and FIG. 95 show the exploded views of this example comprising a spiral spring biasing member 9512 positioned in an inner annular portion of the rotatable member 5804 and retained therein with cover 5808. The spiral spring is attached at one end to the rotatable member 5804 and at the other end to the base 5806. The spiral spring may be configured so its relaxed state corresponds with a particular state of retraction or extension of the tension elements 6802 relative to the rotatable member 5804.

### SIXTH EXAMPLE

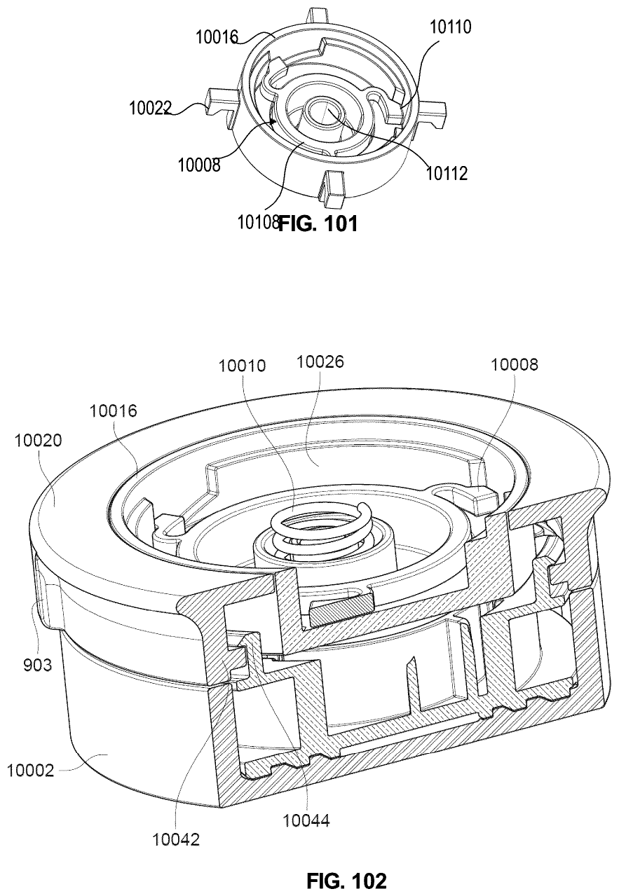

With reference to FIG. 100 to FIG. 105, there is provided a Sixth Example of an adjustment assembly. The adjustment assembly 10018 may include an actuator 10012, a torque transfer element 10008, a rotatable member 10006, one or more biasing members, and a base 10002.

### Actuator

The adjustment assembly 10018 includes an actuator 10012 comprising an outer part 10020 and an inner part 10016 in rotational communication with each other, and configured such that the inner part and outer part may move axially with respect to each other. FIG. 101 shows the inner part 10016 including four equidistantly spaced lugs 10022 configured to slidingly engage with corresponding channels 10024 on the outer part 10020. The outer part 10020 is configured to be directly actuated by the user, and the inner part 10016 comprises the interior contours 10026 that engage with the arms of the torque transfer element 10008. The actuator 10012 operates in much the same way as the actuators described for the First to Fifth Examples, except that it comprises separate inner part 10016 and outer part 10020 that can slide axially along each other, whereas the actuators in the First and Fifth Examples may be unitary bodies, such as integrally formed unitary bodies. The relative axial movement of the inner and outer part enables the actuator to be retained in position relative to the base by a rigid covering cap pin, whilst still allowing the outer part to translate some distance away from the base. Where the outer part is axially coupled to the rotatable member (as discussed below), this allows disengagement of the first and second locking elements by axial movement of the actuator. Alternatively, this may be achieved by an actuator as described for other Examples, in which the axial movement is enabled by an extensible or elastic covering cap pin.

### Torque Transfer Element

As with previous Examples, the torque transfer element 10008 is for receiving torque from the actuator 10012. The torque transfer element 10008 includes a central body 10108 including a central opening 10112, and one or more arms 10110 extending radially from an upper portion of the central body 10108. The torque transfer element 10008 as shown in FIG. 100 to FIG. 105 comprises three arms, although it may include any number of arms, for example, between one and eight. The central opening 10112 is shaped to receive a covering cap 10014 pin therethrough. Each arm 10110 may have substantially the same features as described for the arms in the torque transfer elements of earlier Examples described herein. The arms 10110 and central body 10108 are configured to be received in the interior of the inner part 10016 of the actuator 10012. Accordingly, torque transfer element 10008 has a similar structural design to that of the First to Fourth Examples (for example, that shown in FIG. 4, except that it is not located in a gases flow path). The torque transfer element 10008 further includes one or more legs 10028 extending axially from a lower portion of the central body 10108, configured to connect with the rotatable member 10004 via a central opening. The engagement may be a snap fit connection.

### Rotatable Member

As with previous Examples, the rotatable member 10006 is for winding, storing, and unwinding the tension elements (not shown) about an annular channel on its outer side. Rotatable member 10006 is connected and coaxially positioned with the torque transfer element 10008 in use (e.g., when the parts are connected together). As with the rotatable member of the First and Second Examples, a first locking element 10030 is positioned on the rear side of the rotatable member facing the second locking element 10032 of the base 10002.

### Base

The base 10002 of the Sixth Example comprises a side wall 10034 and a lower wall 10036 defining a base interior that is configured to receive at least part of the rotatable member 10006. One or more openings 10038 in the side wall 10034 are provided to direct one or more tension elements between the rotatable member 10006 and the exterior of the base 10002. A substantially axial post 10040 is located in the center of the lower wall 10036, which is configured to extend towards and mount the rotatable member 10006 so that it may rotate about the post 10040. The post 10040 may be cylindrical or have a C-shaped cross-section, and includes a central vertical recess. It is configured to receive and connect with the covering cap 10014 pin so as to retain the inner member of the actuator 10012, torque transfer element 10008, rotatable first locking element 10030 and biasing members therebetween.

A second locking element 10032 is positioned on the lower wall 10036 of the base 10002, comprising a plurality of teeth for engaging with the first locking element 10030 on the rotatable member 10006, in the engaged configuration. In the engaged configuration, the teeth on the rotatable member intermesh with the teeth on the base. The second locking element is substantially the same as that described in the First and Second Examples, although its annular width may be greater as there is no requirement for a gases flow path to pass through the base in this example.

### Biasing Members

FIG. 100 to FIG. 105 show an example comprising a first biasing member 10010 configured to be positioned in the interior of the actuator 10012 between the covering cap 10014 and the torque transfer element 10008 to exert a rearward biasing force on the torque transfer element 10008 (toward the base 10002).

The adjustment assembly 10018 may be converted from engaged to disengaged configurations in the same manner as with previous Examples, that is, by rotating the actuator 10012 in the untightening direction. The arms of the torque transfer element ride up the ramped contours on the actuator which lifts the first locking element 10030 off the second locking element 10032 and allows rotation of the rotatable member 10006 in the untightening direction and unwinding of the tension element from the rotatable member 10006. The first biasing member 10010 biases the torque transfer element 10008, and by extension the rotatable member 10006, towards the base 10002 and its second locking element 10032. Where the locking elements comprise a plurality of intermeshing teeth, the biasing force on the torque transfer element means that the teeth on the rotatable member slip over the teeth on the base, and the biasing force drives the two sets of teeth into an engaged configuration when they are able to intermesh. Accordingly, the disengaged configuration is transitional between discrete engaged configurations. The increments of unwinding may be accompanied with a tactile or acoustic feedback as the biasing member urges the locking element to snap into an intermeshing engagement with the locking element on the base, which allows the user greater control.

The actuator 10012 also facilitates an additional or alternative mode to obtain a disengaged configuration. The outer part 10020 and rotatable member 10006 are coupled together in the axial direction by a rim 10042 running about the internal side wall of the outer part 10020 that receives a lip 10044 on an upper wall of the rotatable member 10006. Thus, lifting the outer part 10020 axially away from the base 10002 moves the rotatable member 10006 off the second locking element 10032 on the base. The range of axial movement of the outer part 10020 is limited by the covering cap 10014 that pins the inner part 10016 in position relative to the base 10002. A user may find it more intuitive to axially lift the actuator 10012 away from the base 10002 to untighten the adjustment assembly rather than rotate the actuator 10012 in an untightening direction. In this mode of disengagement, the user applies the axial force on the actuator 10012, which is transferred to the rotatable member 10006 by the rim 10042 of the outer part 10020 on the lip 10044 of the rotatable member.

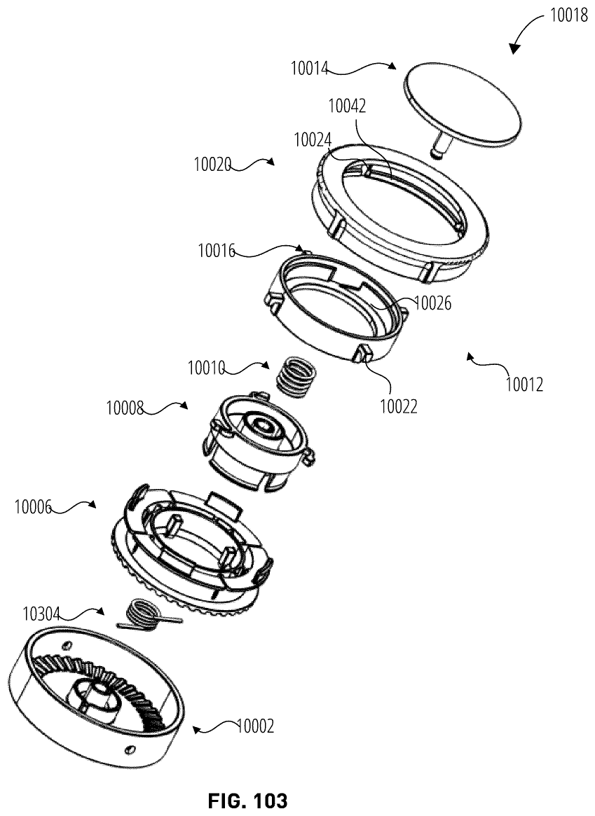

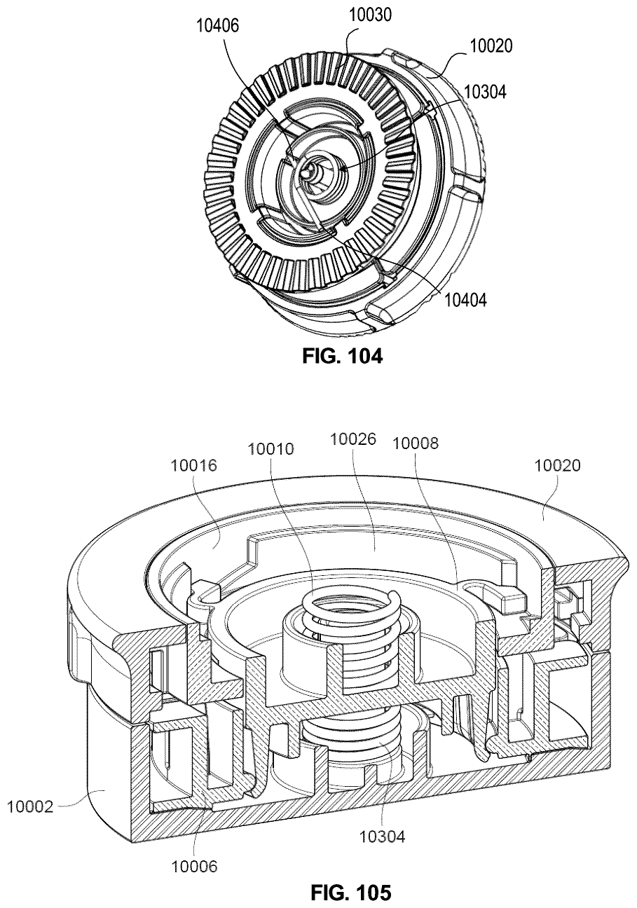

With reference to FIG. 103 to FIG. 105, the adjustment assembly 10018 may further comprise a torsional biasing member 10304 configured to bias the rotatable member 10006 relative to the base 10002 to either a retracted or extended state of tension elements. The torsional biasing member 10304 may be a coil spring surrounding and supported by the base central post, and may be positioned in the central recess of the rotatable member 10006. The coil spring may be connected to, or braced against, the base at a first end 10406 and connected to or braced against the torque transfer element at a second end 10404. In FIG. 104, the coil spring has a first end 10406 braced against a cut-out in the central post of the base 10002, and a second end 10404 braced against the torque transfer element leg 10028. The coil spring may be biased towards an extended or a retracted state.

The adjustment assembly 10018 may be configured to allow the rotatable member 10006 to freely rotate in an untightening direction, e.g., independently of an outer part of the actuator (e.g., part of the adjustment assembly that is gripped by the user like an actuator exterior) when the adjustment assembly is in its disengaged configuration. This may allow the torsional biasing member 10304 to drive the rotatable member towards a state of extension of the tension elements (the particular state of retraction or extension may be pre-configured) without applying a torque onto the actuator or an exterior part thereof.

For example, the actuator may comprise a sleeve for gripping by the user and a core part coupled with the torque transfer element, wherein the core part may rotate relative to the sleeve when the adjustment assembly is in the disengaged configuration. The sleeve may be constructed such that it grips the core when rotated in the tightening direction or when the adjustment assembly is in the engaged configuration, but is rotationally decoupled from the core when the adjustment assembly is in the disengaged configuration.

Alternatively, or additionally, the interior of the actuator may comprise an interior section in which the torque transfer element may rotate independently of the actuator. For example, the interior shown in FIG. 105 may be modified such that the interior of the actuator comprises an arm-interfering contour 10026 and a section in which the contour does not interfere with the torque transfer element arm and in which the torque transfer element may freely rotate. This may be achieved by adapting the interior such that the forward end of the ramp contour leads to a section of the interior in which there is no radial interference on the arms and the torque transfer element may freely rotate. When the adjustment assembly is in the disengaged configuration, a torsional biasing force (e.g., on the rotatable member, by the torsional biasing member 10304) may drive the rotation of the torque transfer element 10008 into this section allowing free rotation in the untightening direction (independently of the actuator). Where this modification is applied to the Sixth Example, the tension elements of a patient interface assembly may be automatically driven towards an extended state simply by a user applying axial movement on the outer part of the actuator.

### Patient Interface Assemblies

The adjustment assembly 10018 is configured to be fitted on a patient interface assembly, for example, on a headgear strap or on a patient interface. The patient interface assembly may include a nasal cannula (contacting or extending into the nares), nasal mask (sealing around the nose), compact nasal mask (sealing around the nares and/or a lower part of the nose), nasal pillows (sealing around or on an inner side of each of the nares), oral mask (sealing around the mouth), full face mask (sealing around the nose and mouth), compact full face mask (combining an oral mask and a nasal mask or nasal pillows), a total face mask (sealing around the eyes, nose and mouth) endotracheal tube (inserted into the trachea through the mouth or nose), tracheostomy tube (inserted into the trachea through an incision in the neck), or other known types of interfaces.

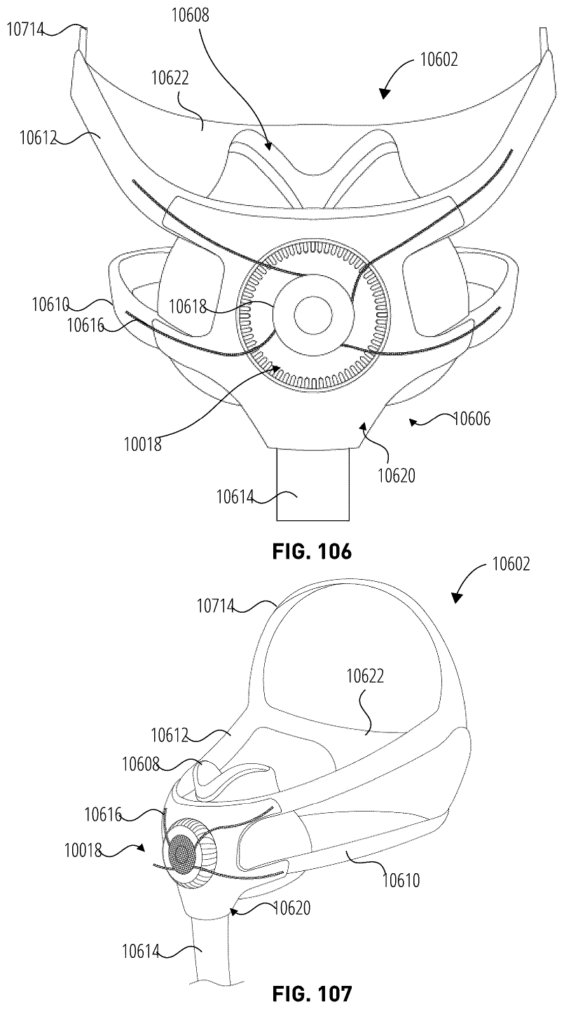

FIG. 106 and FIG. 107 show a patient interface assembly 10602 comprising the adjustment assembly 10018 located on an anterior side of a full-face patient interface 10606 comprising a frame 10620 and flexible seal 10608 configured to seal around the mouth and under the nose and provide breathing gases to both the nares and mouth, including a four-point headgear comprising two upper side straps 10612 and two lower side straps 10610.

The adjustment assembly 10018 may be at least partially formed with a part of the patient interface 10606. For example, the base of the adjustment assembly may be molded onto a rigid portion of the frame 10620. The adjustment assembly 10018 is configured to wind, store, and unwind tension elements extending along left and right upper side straps 10612 and left and right lower side straps 10610. The tension elements 10616 extend out of the adjustment assembly through apertures in the base and extend into at least a portion of each side strap. The configuration of the tension elements in the headgear and/or headgear pathways may be substantially as described above with reference to FIG. 18 to FIG. 25.

Where the tension elements 10616 in the upper side strap 10612 and lower side straps 10610 are wound onto the same part of the rotatable member, rotation of the actuator in the tightening direction causes a substantially similar degree of retraction on each of the four side straps. The patient interface assembly 10602 and adjustment assembly 10018 may be configured to retract the upper side strap 10612 and lower side straps 10610 independently, or at different rates. This may be achieved with a four-point patient interface assembly such as that shown in FIG. 108, comprising a first adjustment assembly 10804 configured to control upper tension elements 10814 in the upper side straps 10810, and a second adjustment assembly 10806 configured to control lower tension elements 10816 in the lower side straps. The first adjustment assembly 10804 is positioned at an upper portion of the frame 10818, proximate a nasal sealing region and approximately laterally aligned with the upper side straps 10810. The second adjustment assembly 10806 is positioned at a lower portion of the frame 10818 or on a breathing gases tube connection 11004, proximate a mouth sealing region and approximately laterally aligned with the lower side straps 10812. Upper tension elements 10814 and lower tension elements 10816 extend from lateral points on the first and second adjustment assemblies towards the respective upper and lower side straps of the headgear. In this example, a user may selectively adjust the tension of the upper side straps and the lower side straps independently of each other.

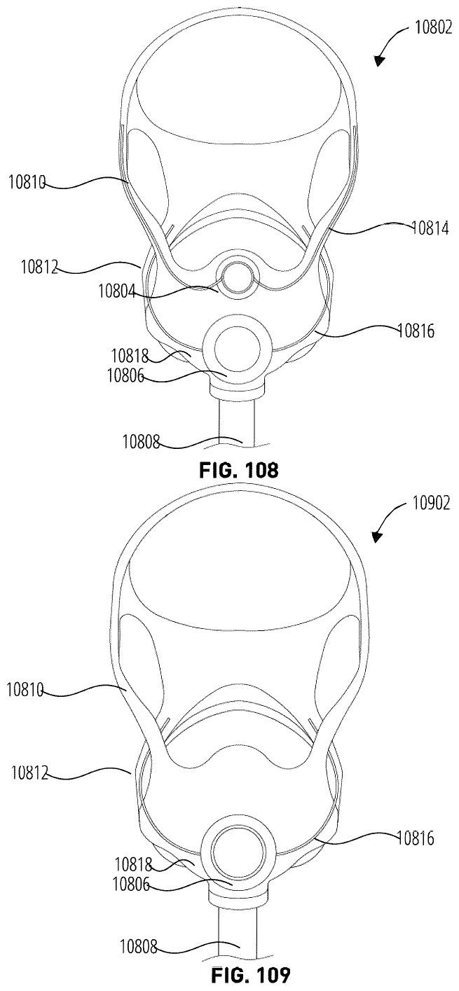

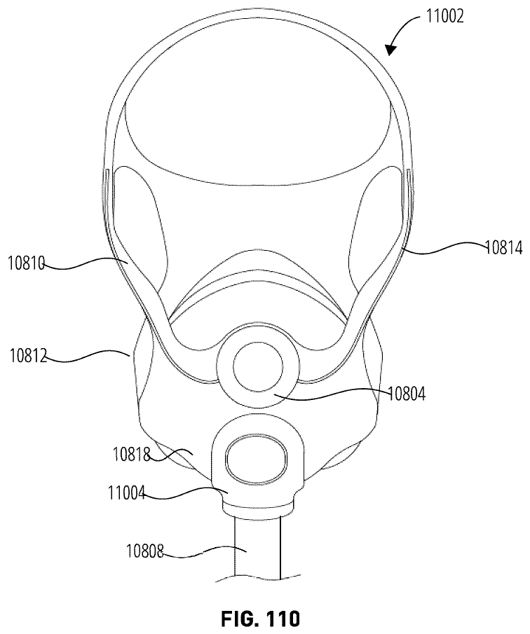

Alternatively, the four-point patient interface assembly may comprise an adjustment assembly configured to control tension elements in the upper or lower side straps, such as the upper straps only, or the lower straps only. FIG. 109 shows a patient interface assembly 10902 in which the adjustment assembly 10806 positioned on a lower portion of the frame 10818 or the breathing gases tube connection 11004 and is configured to control tension in the lower side straps 10812, comprising lower tension elements 10816 extending along a lower portion of the frame 10818 towards the lower side straps 10812. The tension in and/or length of the upper side straps may be adjusted manually, such as by a buckle arrangement in each of the upper side straps. FIG. 110 shows a patient interface assembly 11002 positioned on an upper portion of the frame 10818 and is configured to control tension in the upper side straps 10810, comprising tension elements extending along an upper portion of the frame 10818 towards the upper side straps 10810. The tension in and/or length of the lower side straps may be adjusted manually, such as by a buckle arrangement in each of the lower side straps.

Alternatively, separate tension elements may be located on a single adjustment assembly and differently geared by configuring the rotatable member and tension elements so that a tension element may wind about the rotatable member at different diameters.

1-153. (canceled)

154. A patient interface assembly for supplying a flow of pressurized breathing gases to an airway of a patient, the patient interface assembly comprising:
a patient interface comprising a cushion configured to seal around a portion of a face of the patient;
a headgear assembly, comprising:
at least two side straps; and
one or more tension elements configured for engagement with the at least two side straps;
at least one adjustment assembly, comprising:
a rotatable member coupled with the one or more tension elements;
an actuator configured to selectively rotate the rotatable member to adjust a tension in the one or more tension elements; and
a torque transfer element configured to communicate torque between the actuator and the rotatable member, and configured to limit the torque applied to the rotatable member by the actuator.

155. The patient interface assembly of claim 154, wherein the torque transfer element is configured to limit the torque applied to the rotatable member by the actuator to a torque threshold.

156. The patient interface assembly of claim 155, wherein the actuator and the torque transfer element are rotationally coupled up to the torque threshold, and rotationally decoupled above the torque threshold.

157. The patient interface assembly of claim 154, wherein each of the one or more tension elements extend into at least a portion of a respective one of the at least two side straps.

158. The patient interface assembly of claim 154, wherein the at least one adjustment assembly is configured to wind, store and unwind the one or more tension elements.

159. The patient interface assembly of claim 154, wherein:
the at least one adjustment assembly comprises one or more apertures through which each of the one or more tension elements extend, and
the rotatable member comprises an annular channel into which each of the one or more tension elements may be wound, stored and/or unwound.

160. The patient interface assembly of claim 154, wherein the at least one adjustment assembly is at least partially formed with a part of the patient interface.

161. The patient interface assembly of claim 154, wherein the at least two side straps comprise a pair of upper side straps and a pair of lower side straps.

162. The patient interface assembly of claim 161, wherein the tension of the one or more tension elements in engagement with the pair of upper side straps is independent of the tension of the one or more tension elements in engagement with the pair of lower side straps.

163. The patient interface assembly of claim 161, wherein:
the at least one adjustment assembly comprises a first adjustment assembly, and the pair of upper side straps and pair of lower side straps are both associated with the first adjustment assembly, or
the at least one adjustment assembly comprises the first adjustment assembly and a second adjustment assembly, and the pair of upper side straps are associated with the first adjustment assembly, and the pair of lower side straps are associated with the second adjustment assembly.

164. The patient interface assembly of claim 154, wherein the torque transfer element is configured to move axially and rotationally with respect to the actuator.

165. The patient interface assembly of claim 154, wherein the headgear assembly comprises a first locking element and a second locking element biased against each other.

166. The patient interface assembly of claim 165, wherein the first locking element is positioned on the rotatable member or on the torque transfer element, and a base of the at least one adjustment assembly comprises the second locking element.

167. The patient interface assembly of claim 154, wherein the at least one adjustment assembly comprises a base that is molded onto a frame of the patient interface.

168. The patient interface assembly of claim 154, wherein the actuator comprises an inner part configured to engage with the torque transfer element and an outer part configured to be directly actuated by a user, the inner part and the outer part are in rotational communication with each other.

169. The patient interface assembly of claim 154, wherein rotation of the actuator in a tightening direction drives rotation of the rotatable member via a torque transfer mechanism comprising the torque transfer element, and a retracting action on the one or more tension elements, such that the headgear assembly is tightened.

170. The patient interface assembly of claim 169, wherein the at least two side straps comprises four side straps, wherein the rotation of the actuator in the tightening direction causes a substantially similar degree of retraction on each of the four side straps.

171. The patient interface assembly of claim 154, wherein the torque transfer element is biased towards a rear end of the actuator, such that torque is transferrable from the actuator to the rotatable member via the torque transfer element.

172. The patient interface assembly of claim 171, wherein:
rotation of the actuator in an untightening direction urges axial movement of the torque transfer element away from the rear end of the actuator,
axial movement of the torque transfer element away from the rear end of the actuator transfers to the rotatable member, and
axial movement of the rotatable member urges a first locking element and a second locking element away from each other.

173. The patient interface assembly of claim 154, wherein when a first locking element and a second locking element are disengaged, the headgear assembly can be loosened by rotating the actuator in an untightening direction, or by applying tension to the headgear assembly to pull the one or more tension elements from the rotatable member.
